(19) United States
(12) Patent Application Publication
(10) Pub. No.: US 2025/0049747 A1
(43) Pub. Date: Feb. 13, 2025

(54) USE OF HYPERPOLARIZING AGENTS ALONE AND IN COMBINATION WITH OTHER THERAPEUTIC AGENTS FOR TREATING CANCERS INCLUDING GLIOBLASTOMA

(71) Applicant: Trustees of Tufts College, Medford, MA (US)

(72) Inventors: Michael Levin, Medford, MA (US); Juanita Mathews, Medford, MA (US)

(21) Appl. No.: 18/719,825

(22) PCT Filed: Dec. 14, 2022
(86) PCT No.: PCT/US2022/081589

§ 371 Date: Jun. 13, 2024

Related U.S. Application Data
(60) Provisional application No. 63/265,403, filed on Dec. 14, 2021.

(51) Int. Cl. A61K 31/27; A61K 31/166; A61K 31/17; A61K 31/196; A61K 31/216; A61K 31/422; A61K 31/423; A61K 31/436; A61K 31/4439; A61K 31/506; A61K 31/53; A61K 31/573; A61K 31/7048; A61P 35/00

(52) U.S. Cl. CPC A61K 31/27; A61K 31/166; A61K 31/17; A61K 31/196; A61K 31/216; A61K 31/422; A61K 31/423; A61K 31/436; A61K 31/4439; A61K 31/506; A61K 31/53; A61K 31/573; A61K 31/7048; A61P 35/00

(57) ABSTRACT

Disclosed are methods and compositions for treating cell proliferative diseases and disorders including cancers such as glioblastoma. The disclosed methods and compositions may utilize or comprise one or more modulators of the bioelectric state of cells, such as, one or more potassium channel activators; a sodium channel inhibitor combined with an mTOR inhibitor; a sodium channel inhibitor combined with a proton pump inhibitor; a calcium channel inhibitor combined with an mTOR inhibitor; a proton pump inhibitor combined with an alkylating agent; or a combination of at least one potassium channel activator and one or more of an alkylating agent, calcium channel inhibitor, corticosteroid, mTOR inhibitor, proton pump inhibitor, and/or sodium channel inhibitor.

### CROSS-REFERENCE TO RELATED APPLICATIONS

This application claims priority to U.S. Provisional Application No. 63/265,403 filed on Dec. 14, 2021, the contents of which are incorporated by reference in its entirety.

### BACKGROUND

The field of the invention relates to methods and compositions for treating cell proliferative diseases and disorders such as cancer. In particular, the field of the invention relates to methods and compositions for treating brain cancers such as glioblastoma in a subject by administering to the subject one or more therapeutic agents that modulate the polarity of glioblastoma cells.

Glioblastoma is a highly lethal cancer that can reoccur after the initial resection of the tumor due to a population of glioblastoma stem cells just outside the border of the main mass. It is thus critical to identify ways of keeping these cancer cells from proliferating by inducing cell cycle arrest. Depolarized resting membrane potentials have been associated with a proliferative state, suggesting that forced hyperpolarization could be a strategy for targeting glioblastoma cell overgrowth.

To discover potentially useful modulators of bioelectric state, the present inventors used NG108-15 cells and U87 cells in high serum media as a model to screen ion channel-modulating drugs, by themselves and in combination with each other or with compounds known to be somewhat effective at reducing GBM proliferation. In particular, the inventors tested whether drugs or drug combinations that could efficiently hyperpolarize cells would be capable of significantly reducing the proliferation of NG108-15 cells, and terminally differentiating NG108-15 cells.

As disclosed herein, the inventors found that several agents that modulate the activity of membrane channels, and combinations thereof, were very effective at reducing the proliferation of cells during treatment, and this effect persisted days after treatment was discontinued. The inventors' findings have implications for treating cancers that express membrane channels accordingly.

### SUMMARY

Disclosed are methods and compositions for treating cell proliferative diseases and disorders such as glioblastoma. The disclosed compositions comprise and the methods utilize one or more therapeutic agents that modulate the polarity of a cancer cell and may include therapeutic agents that hyperpolarize the cell membrane and modulate cancer differentiation and/or growth. Cancers that may be treated by the disclosed compositions and methods include glioblastomas.

In one aspect, methods for treating a cell proliferative disease or disorder in a subject in need thereof are provided. The methods include administering an effective amount of one or more potassium channel activators to the subject. The potassium channel activator may be administered in combination with one or more of a mTOR inhibitor, an alkylating agent, a corticosteroid, a proton pump inhibitor, a sodium channel inhibitor, a calcium channel inhibitor or a peroxisome proliferator-activated receptor alpha (PPARα) activator.

In another aspect, methods for treating a cell proliferative disease or disorder in a subject in need thereof, which include (i) administering to the subject an effective amount of a sodium channel inhibitor (e.g., a sodium channel blocker); and (ii) administering to the subject an effective amount of a mTOR inhibitor are provided. The sodium channel inhibitor is administered to the subject before, concurrently with, or after the mTOR inhibitor is administered to the subject.

In a further aspect, methods for treating a cell proliferative disease or disorder in a subject in need thereof, which include (i) administering to the subject an effective amount of a sodium channel inhibitor (e.g., a sodium channel blocker); and (ii) administering to the subject an effective amount of a proton pump inhibitor are provided. The sodium channel inhibitor is administered to the subject before, concurrently with, or after the proton pump inhibitor is administered to the subject.

In a still further aspect, methods for treating a cell proliferative disease or disorder in a subject in need thereof, which includes (i) administering to the subject an effective amount of a calcium channel inhibitor (e.g., a calcium channel blocker); and (ii) administering to the subject an effective amount of a mTOR inhibitor are provided. The calcium channel inhibitor is administered to the subject before, concurrently with, or after the mTOR inhibitor is administered to the subject.

In yet another aspect, methods for treating a cell proliferative disease or disorder in a subject in need thereof, which include (i) administering to the subject an effective amount of a proton pump inhibitor; and (ii) administering to the subject an effective amount of an alkylating agent are provided. The proton pump inhibitor is administered to the subject before, concurrently with, or after the alkylating agent is administered to the subject

### BRIEF DESCRIPTION OF THE FIGURES

FIG. 1: NG108-15 Proliferation is Significantly Lowered with Bioelectric Treatment and Show Changes in Cell Cycle Ratios. (A) Fold change to start cell counts (cells at day6/cells at day0) after 6 days of treatment. Low values indicate less cell growth. Colors indicate treatments followed up for further analysis. Red shaded treatments correspond to positive controls that cannot be used clinically. Only treatments with significant values are shown out of 33 treatments compared to DMSO control. ****:q<0.0001 ***:q<0.001, **:q<0.01, *:q<0.05 (one-way ANOVA with FDR post hoc analysis n>3 biological replicates). (B) FUCCI cell cycle data at day 6. Increased red and orange fractions indicate cell cycle arrest at G1 or G1 to S transition.

FIG. 2: Combinations of Pantoprazole with Bioelectric Compounds Significantly Decrease Proliferation Compared to Pantoprazole Alone and Show Changes in Cell Cycle Ratio. (A) Percent reduction in cells compared to control after 6 days of treatment. Treatments that were significantly more effective than pantoprazole alone are shown out of 32 treatments. Statistical analysis was done on the log 2 of the fold change in cell number to control on day 6. ***:q<0.001, **:q<0.01, *:q<0.05 (one-way ANOVA with FDR post hoc analysis n>3 biological replicates). (B) FUCCI cell cycle data at day 6. Increased red and orange fractions indicate cell cycle arrest at G1 or G1 to S transition.

FIG. 3: Recovery Test of Hyperpolarizing Treatments in Combination with Pantoprazole in NG108-15 FUCCI Cells. The log 2 of the fold change in cell counts to Day 0 were recorded for 10 days. Dotted line marks the day on which drug treatment was removed and replaced with control media (n>3 biological replicates). Combination drug treatment slopes from Day 6 to Day 10 were compared to pantoprazole alone, significance is shown with grey stars next to the corresponding line **:p<0.01, *:p<0.05 (one-way ANOVA with Dunnett post hoc analysis n>3 biological replicates).

FIG. 4: Hyperpolarizing Drugs in Combination with Each Other, Pantoprazole, and TMZ Reduced Proliferation in U87 cells and Changed the Cell Cycle Ratio Compared to Control. (A) Fold change to start cell counts (cells at day6/cells at day0) after 6 days of treatment. Low values indicate less cell growth. Colors indicate treatments followed up for further analysis. Red shaded treatment corresponds to positive control that cannot be used clinically. Only treatments with significant values are shown out of 42 treatments compared to DMSO control. ****:q<0.0001, ***:q<0.001, **:q<0.01, *:q<0.05 (one-way ANOVA with FDR post hoc analysis n>3 biological replicates). (B) FUCCI cell cycle data at day 6. Increased red and orange fractions indicate cell cycle arrest at G1 or G1 to S transition.

FIG. 5: Treatments with Hyperpolarizing Compounds and Pantoprazole or TMZ were Significantly Better than TMZ Alone at Reducing Proliferation in U87 cells and Changed the Cell Cycle Ratio Compared to Control. (A) Percent reduction in cells compared to control after 6 days of treatment. Treatments that were significantly more effective than TMZ alone are shown out of 42 treatments. Red shaded treatment corresponds to positive control that cannot be used clinically. Statistical analysis was done on the log 2 of the fold change in cell number to control on day 6. ***:q<0.001, **:q<0.01, *:q<0.05 (one-way ANOVA with FDR post hoc analysis n>3 biological replicates). (B) FUCCI cell cycle data at day 6. Increased red and orange fractions indicate cell cycle arrest at G1 or G1 to S transition.

FIG. 6: Combination of Hyperpolarizing Drugs with Pantoprazole or TMZ or Pantoprazole with TMZ Showed the Most Significant Decrease in Cell Proliferation and Changed the Cell Cycle Ratio Compared to Control (A) Percent reduction in cells compared to control after 6 days of treatment. Treatments that were significantly more effective than Pantoprazole alone are shown out of 42 treatments. Statistical analysis was done on the log 2 of the fold change in cell number to control on day 6. ***:q<0.001, **:q<0.01, *:q<0.05 (one-way ANOVA with FDR post hoc analysis n>3 biological replicates). (B) FUCCI cell cycle data at day 6. Increased red and orange fractions indicate cell cycle arrest at G1 or G1 to S transition.

FIG. 7: Recovery Test of Hyperpolarizing Treatments in Combination with Pantoprazole or TMZ. The log 2 of the fold change in cell counts to Day 0 were recorded for 10 days. Dotted line marks the day on which drug treatment was removed and replaced with control media (n>3 biological replicates). Combination drug treatment slopes from Day 6 to Day 10 were compared to pantoprazole or TMZ alone, but no significance was found (one-way ANOVA with Dunnett post hoc analysis n>3 biological replicates).

FIG. 8: Resting Membrane Potential Changes Caused by Treatments in NG108-15 FUCCI Palmitoyl-mTurquoise2 Cells. The change in resting membrane potential as normalized to DMSO control. The more negative values show an increase in hyperpolarization. (A) and (B) were done on different days. The sample size n=55-63 cells per condition. Significant values shown in tables. ****: p<0.0001, were calculated using an ANOVA with Dunnet post-hoc analysis.

FIG. 9: Differentiation Analysis of NG108-15 Cells Reveals that Treatments with Pantoprazole Increased Neuronal Markers after 6 days. Immunofluorescence of cells was analyzed with CellProfiler and quantified for integrated fluorescence intensity. (A) Stain of Microtubule Associated Protein 2 (MAP2). (B) Stain of Neuron-Specific Class III beta-Tubulin (Tuj I). (C) Stain of Neural Filament Medium Chain (NFM). (D) Stain of Neuron-Specific Enolase (NSE). The log of the fold change in intensity was compared between single treatments and combined treatments, except the positive control, with significant values shown in the color of the treatment compared. The initial fluorescence intensities were compared to their corresponding control, with significant values shown under the bars as, ***:p<0.001, **:p<0.01, *:p<0.05 (one-way ANOVA with Tukey post hoc analysis n>3 technical replicates).

FIG. 10: Differentiation Analysis of NG108-15 Cells Reveals that Treatments with Pantoprazole Increased Astrocytic and Differentiation Markers after 6 days. Immunofluorescence of cells was done and analyzed with CellProfiler and measured for integrated fluorescence intensity. (A) Stain of S100 calcium binding protein B (S100B). (B) Stain Glial Fibrillary Acidic Protein (GFAP). (C) Stain of the phosphorylated cAMP-Response Element Binding Protein (Phospho CREB). (D) Stain of Connexin 43 (Cx43). The log of the fold change in intensity was compared between single treatments and combined treatments, except the positive control, with significant values shown in the color of the treatment compared. The initial fluorescence intensities were compared to their corresponding control, with significant values shown under the bars as, ***:p<0.001, **:p<0.01, *:p<0.05 (one-way ANOVA with Tukey post hoc analysis n>3 technical replicates).

FIG. 11: Senescence and Proliferation Analysis of NG108-15 Cells Reveals that Treatments with Pantoprazole Increased Senescence, Decreased BrdU Incorporation, and Increased a p27^{Kip1} after 6 days. A senescence associated beta-galactosidase stain was done and scored by eye. Immunofluorescence of cells was done and analyzed with CellProfiler for integrated fluorescence intensity or presence or absence of a cellular signal. (A) Stain of senescence associated beta-galactosidase stain (SA-Beta Gal). (B) Stain of bromodeoxyuridine incorporation (BrdU). (C) Stain of the microtubule-associated protein light chain 3 II (LC3-II). (D) Stain of cleaved caspase 3 (Casp 3). (E) Stain of cyclin-dependent kinase inhibitor 1B (p27Kip). (F) Size of Nuclei, determined by area of the Hoechst stain. The log of the fold change in intensity was compared between single treatments and combined treatments, except the positive control, with significant values shown in the color of the treatment compared. The initial fluorescence intensities were compared to their corresponding control, with significant values shown under the bars. The logit of the percent positive cells was compared between single treatments and control, in cases of 0 values, the arcsine transformation was used. Significance was expressed as, ***:p<0.001, **:p<0.01, *:p<0.05 (one-way ANOVA with Tukey post hoc analysis n>3 technical replicates).

FIG. 12: Differentiation Analysis of U87 Cells Reveals that Treatments with Pantoprazole Increased Neuronal Markers after 6 days. Immunofluorescence of cells was done and analyzed with CellProfiler for integrated fluorescence intensity. (A) Stain of Microtubule Associated Protein 2 (MAP2). (B) Stain of Neuron-Specific Class III beta-Tubulin (Tuj I). (C) Stain of Neural Filament Medium Chain (NFM). (D) Stain of Neuron-Specific Enolase (NSE). The log of the fold change in intensity was compared between single treatments and combined treatments, except the positive control, with significant values shown in the color of the treatment compared. The initial fluorescence intensities were compared to their corresponding control, with significant values shown under the bars as, ***:p<0.001, **:p<0.01, *:p<0.05 (one-way ANOVA with Tukey post hoc analysis n>3 technical replicates).

FIG. 13: Differentiation Analysis of U87 Cells Reveals that Treatments with Pantoprazole Increased Astrocytic and Differentiation Markers after 6 days. Immunofluorescence of cells was done and analyzed with CellProfiler for integrated fluorescence intensity. (A) Stain of Vimentin. (B) Stain of the phosphorylated cAMP-Response Element Binding Protein (CREB). (C) Stain of S100 calcium binding protein B (S100B). (D) Stain Glial Fibrillary Acidic Protein (GFAP). The log of the fold change in intensity was compared between single treatments and combined treatments, except the positive control, with significant values shown in the color of the treatment compared. The initial fluorescence intensities were compared to their corresponding control, with significant values shown under the bars as, ***:p<0.001, **:p<0.01, *:p<0.05 (one-way ANOVA with Tukey post hoc analysis n>3 technical replicates).

FIG. 14: Differentiation Analysis of U87 Cells Reveals that Treatments with Pantoprazole Increased Oligodendrocyte Markers after 6 days. Immunofluorescence of cells was done and analyzed with CellProfiler for integrated fluorescence intensity. (A) Stain of oligodendrocyte marker 04. (B) Stain of the Sry-related HMg-Box gene 10 (SOX10). The log of the fold change in intensity was compared between single treatments and combined treatments, except the positive control, with significant values shown in the color of the treatment compared. The initial fluorescence intensities were compared to their corresponding control, with significant values shown under the bars as, ***:p<0.001, **:p<0.01, *:p<0.05 (one-way ANOVA with Tukey post hoc analysis n>3 technical replicates).

FIG. 15: Senescence and Proliferation Analysis of U87 Cells Reveals that Treatments with Pantoprazole or NS164 with TMZ Increased Senescence, Decreased BrdU Incorporation, and Increased a p27^{K}‘P’ after 6 days A senescence associated beta-galactosidase stain was done and scored by eye. Immunofluorescence of cells was done and analyzed with CellProfiler for integrated fluorescence intensity or presence or absence of a cellular signal. (A) Stain of senescence associated beta-galactosidase stain (SA-Beta Gal). (B) Stain of bromodeoxyuridine incorporation (BrdU). (C) Stain of the microtubule-associated protein light chain 3 II (LC3-II). (D) Stain of cleaved caspase 3 (Casp 3). (E) Stain of cyclin-dependent kinase inhibitor 1B (p27Kip). (F) Size of Nuclei, determined by area of the Hoechst stain. The log of the fold change in intensity was compared between single treatments and combined treatments, except the positive control, with significant values shown in the color of the treatment compared. The initial fluorescence intensities were compared to their corresponding control, with significant values shown under the bars. The logit of the percent positive cells was compared between single treatments and control, in cases of 0 values, the arcsine transformation was used. Significance was expressed as, ***:p<0.001, **:p<0.01, *:p<0.05 (one-way ANOVA with Tukey post hoc analysis n>3 technical replicates).

FIG. 16: Voltage Dyes Showed that U87 Cells Treated with NS1643 and a Combination of NS1643 and Pantoprazole for 6 Days Showed a Hyperpolarization and YAP Increases its Translocation to the Cytoplasm in NS1643 or Pantoprazole with TMZ, and also Pantoprazole with NS1643 Treatment. Immunofluorescence of cells was done and analyzed with CellProfiler for integrated fluorescence intensity. Dye assays were analyzed for mean intensity, except for LysoSensor Green which was analyzed for integrated intensity. (A) Stain of lysosomal pH with LysoSensor Green, low levels indicate alkalization. (B) Dye indicator of membrane voltage, DiBAC4(3), low levels indicate hyperpolarization. (C) Dye indicator of cytoplasmic pH, pHRodo Green, low levels indicate alkalization. (D) Dye indicator of cytoplasmic calcium, Fluo-4AM, high levels indicate an increase in calcium. (E) The ratio of nuclear to cytoplasmic Yes-associated protein (YAP), lower levels indicate translocation to the cytoplasm. The log of the fold change in intensity was compared between single treatments and combined treatments, except the positive control, with significant values shown in the color of the treatment compared. The initial fluorescence intensities were compared to their corresponding control, with significant values shown under the bars as, ***:p<0.001, **:p<0.01, *:p<0.05 (one-way ANOVA with Tukey post hoc analysis n>3 technical replicates).

FIG. 17: Live/Dead assay and Senescence assay of Human Neuronal Cells After 3 Day Treatment Shows Low Level of Toxicity. Low values indicate less death or senescent cells. (A) Live/Dead assay done on human neuronal cells cultured with drug for 3 days. (B) Senescence assay results of senescence associated beta-galactosidase staining on human neuronal cells cultured with drug for 3 days. Treatments with best reduction of proliferation in NG108-15 or U87 cells are shown out of a 24-sample toxicity screen, with significant values shown. ***:q<0.001, **:q<0.01, *:q<0.05 (one-way ANOVA with FDR post hoc analysis n>3 technical replicates). Increase in percent dead or senescent cells indicative of toxic treatment.

FIG. 18: NG108-15 Initial Screen of Compounds Part A. Percent reduction in cells compared to control after 6 days of treatment, n>5 technical replicates. Colored plots indicate compounds that were analyzed later in NG108-15 or U87 cells.

FIG. 19: NG108-15 Initial Screen of Compounds Part B. Percent reduction in cells compared to control after 6 days of treatment, n>5 technical replicates. Colored plots indicate compounds that were analyzed later in NG108-15 or U87 cells. Red shaded treatments are positive controls used in later analysis.

FIG. 20: U87 Initial Screen of Compounds. Percent reduction in cells compared to control after 6 days of treatment, n>5 technical replicates. Colored plots indicate compounds that were analyzed later in NG108-15 or U87 cells. Red shaded treatments are positive controls used in later analysis.

FIGS. 21-51 show results of the tumor tissue cytotoxicity assay for various drug treatments on both colorectal cancer and breast cancer tissues. FIGS. 21-22 show the overall impact scores based on viability and morphology of cancer cells in the treated tissues on a scale of 0-100 as assessed by various parameters, including by nuclear details, tissue cohesiveness, cytoplasmic changes, and immunohistochemistry staining. On this scale, a higher score correlates with improved clinical responses and scores lower than 30 are considered as no response. The treatments tested were six days of (1) 100 μM pantoprazole combined with 10 μM retigabine, (2) 100 μM pantoprazole combined with 100 μM of Lamotrigine, (3) 200 nM rapamycin combined with 10 μM retigabine, (4) 200 nM rapamycin combined with 30 μM minoxidil, (5) 200 nM rapamycin combined with 10 μM Zolmitriptan, (6) 200 nM rapamycin combined with 50 μM NS1643, and (7) 50 μM of NS1643.

FIG. 21: “CRC” refers to colorectal cancer cell tissues and “breast” refers to breast cancer cell tissues.

FIG. 22: “Median” refers to the median impact score of all drug treatments tested.

FIGS. 23-51 show histological results of the tumor tissue cytotoxicity assay for the various drug treatments on both colorectal cancer and breast cancer tissues as detailed below.

FIG. 23 shows hematoxylin and eosin (H&E) staining histological results of the tumor tissue cytotoxicity assay for colorectal cancer tissue treated with various treatments as labeled, including an untreated control (bottom, far right).

FIG. 24: H&E staining histological results of the tumor tissue cytotoxicity assay for colorectal cancer tissue treated with various treatments including an untreated control (bottom, far right). (NT=no tumor)

FIG. 25: H&E staining histological results of the tumor tissue cytotoxicity assay for colorectal cancer tissue treated with various treatments including an untreated control (bottom, far right).

FIG. 26: H&E staining histological results of the tumor tissue cytotoxicity assay for breast cancer tissue treated with various treatments including an untreated control (bottom, far right).

FIG. 27: H&E staining histological results of the tumor tissue cytotoxicity assay for breast cancer tissue treated with various treatments including an untreated control (bottom, far right). (NT=no tumor)

FIG. 28: H&E staining histological results of the tumor tissue cytotoxicity assay for breast cancer tissue treated with various treatments including an untreated control (bottom, far right). (NA=not applicable for technical reasons)

FIG. 29: H&E staining histological results of the tumor tissue cytotoxicity assay for colorectal cancer tissue treated with pantoprazole combined with retigabine (top) or pantoprazole combined with lamotrigine (bottom).

FIG. 30: H&E staining histological results of the tumor tissue cytotoxicity assay for colorectal cancer tissue treated with rapamycin combined with retigabine (top) or rapamycin combined with minoxidil (bottom).

FIG. 31: H&E staining histological results of the tumor tissue cytotoxicity assay for colorectal cancer tissue treated with rapamycin combined with zolmitriptan (top) or rapamycin combined with NS1643 (bottom).

FIG. 32: H&E staining histological results of the tumor tissue cytotoxicity assay for colorectal cancer tissue treated with NS1643 alone (top) or untreated as a control (bottom).

FIG. 33: H&E staining histological results of the tumor tissue cytotoxicity assay for colorectal cancer tissue treated with pantoprazole combined with retigabine (top) or pantoprazole combined with lamotrigine (bottom).

FIG. 34: H&E staining histological results of the tumor tissue cytotoxicity assay for colorectal cancer tissue treated with rapamycin combined with retigabine (top) or rapamycin combined with minoxidil (bottom).

FIG. 35: H&E staining histological results of the tumor tissue cytotoxicity assay for colorectal cancer tissue treated with rapamycin combined with zolmitriptan (top) or rapamycin combined with NS1643 (bottom). (NT=no tumor)

FIG. 36: H&E staining histological results of the tumor tissue cytotoxicity assay for colorectal cancer tissue treated with NS1643 alone (top) or untreated as a control (bottom).

FIG. 37: H&E staining histological results of the tumor tissue cytotoxicity assay for colorectal cancer tissue treated with pantoprazole combined with retigabine (top) or pantoprazole combined with lamotrigine (bottom).

FIG. 38: H&E staining histological results of the tumor tissue cytotoxicity assay for colorectal cancer tissue treated with rapamycin combined with retigabine (top) or rapamycin combined with minoxidil (bottom).

FIG. 39: H&E staining histological results of the tumor tissue cytotoxicity assay for colorectal cancer tissue treated with rapamycin combined with zolmitriptan (top) or rapamycin combined with NS1643 (bottom).

FIG. 40: H&E staining histological results of the tumor tissue cytotoxicity assay for colorectal cancer tissue treated with NS1643 alone (top) or untreated as a control (bottom).

FIG. 41: H&E staining histological results of the tumor tissue cytotoxicity assay for breast cancer tissue treated with pantoprazole combined with retigabine (top) or pantoprazole combined with lamotrigine (bottom).

FIG. 42: H&E staining histological results of the tumor tissue cytotoxicity assay for breast cancer tissue treated with rapamycin combined with retigabine (top) or rapamycin combined with minoxidil (bottom).

FIG. 43: H&E staining histological results of the tumor tissue cytotoxicity assay for breast cancer tissue treated with rapamycin combined with zolmitriptan (top) or rapamycin combined with NS1643 (bottom).

FIG. 44: H&E staining histological results of the tumor tissue cytotoxicity assay for breast cancer tissue treated with NS1643 alone (top) or untreated as a control (bottom).

FIG. 45: H&E staining histological results of the tumor tissue cytotoxicity assay for breast cancer tissue treated with pantoprazole combined with retigabine (top) or pantoprazole combined with lamotrigine (bottom).

FIG. 46: H&E staining histological results of the tumor tissue cytotoxicity assay for breast cancer tissue treated with rapamycin combined with retigabine (top) or rapamycin combined with minoxidil (bottom).

FIG. 47: H&E staining histological results of the tumor tissue cytotoxicity assay for breast cancer tissue treated with rapamycin combined with zolmitriptan (top) or rapamycin combined with NS1643 (bottom). (NT=no tumor)

FIG. 48: H&E staining histological results of the tumor tissue cytotoxicity assay for breast cancer tissue treated with NS1643 alone (top) or untreated as a control (bottom). (NT=no tumor)

FIG. 49: H&E staining histological results of the tumor tissue cytotoxicity assay for breast cancer tissue treated with pantoprazole combined with retigabine (top) or pantoprazole combined with lamotrigine (bottom).

FIG. 50: H&E staining histological results of the tumor tissue cytotoxicity assay for breast cancer tissue treated with rapamycin combined with retigabine (top) or rapamycin combined with minoxidil (bottom).

FIG. 51: H&E staining histological results of the tumor tissue cytotoxicity assay for breast cancer tissue treated with rapamycin combined with NS1643.

### DETAILED DESCRIPTION

The disclosed subject matter further may be described utilizing terms as defined below.

Unless otherwise specified or indicated by context, the terms “a”, “an”, and “the” mean “one or more.” For example, “a therapeutic agent” should be interpreted to mean “one or more therapeutic agents.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

The terms “subject,” “patient,” and “individual” may be used interchangeably herein. A subject may be a human subject. A subject may refer to a human subject having or at risk for acquiring a cell proliferative disease or disorder such as cancers including, but not limited to brain cancer (e.g., glioblastoma multiforme (GBM)), prostate cancer, breast cancer, lung cancer (e.g., non-small cell lung cancer (NSCLC)), colorectal cancer, bladder cancer, kidney cancer, uterine cancer, melanoma, lymphoma (e.g., Non-Hodgkin lymphoma), leukemia, pancreatic cancer, ovarian cancer, liver and intrahepatic bile duct cancer, oral cavity cancer, and esophageal cancer, and in particular, brain cancers such as glioblastoma.

Use of Hyperpolarizing Agents Alone and in Combination with Other Therapeutic Agents for Treating Cancers

The disclosed subject matter relates to compositions and methods for treating cell proliferative diseases or disorders in a subject in need thereof. The compositions comprise and the methods utilize one or more therapeutic agents that modulate the polarity of a cancers cell and may include therapeutic agents that hyperpolarize the cell membrane and modulate cancer differentiation and/or growth.

In some embodiments, the disclosed subject matter relates to methods for treating a cell proliferative disease or disorder in a subject in need thereof. The treatment methods may include administering to the subject an effective amount of one or more potassium channel activators.

Suitable potassium channel activators for use in the disclosed treatment methods may include, but are not limited to, agents that activate the KCNQ/Kv7 channel. Suitable potassium channel activators may include, but are not limited to retigabine having the following chemical structure or suitable pharmaceutical salts thereof:

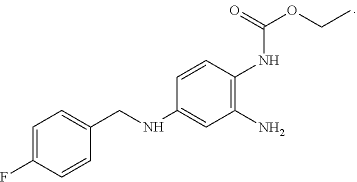 [SMILES: CCOC(=O)Nc1ccc(NCc2ccc(F)cc2)cc1N]

Suitable potassium channel activators for use in the disclosed treatment methods may include, but are not limited to, agents that activate the K(ATP) channel. Suitable potassium channel activators may include, but are not limited to, minoxidil having the following chemical structure or suitable pharmaceutical salts thereof:

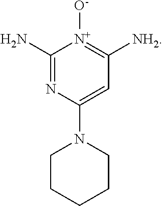 [SMILES: Nc1cc(N2CCCCC2)nc(N)[n+]1[O-]]

Suitable potassium channel activators for use in the disclosed treatment methods may include, but are not limited to, agents that activate the hERG channel. Suitable potassium channel activators may include, but are not limited to, NS1643 having the following chemical structure or suitable pharmaceutical salts thereof:

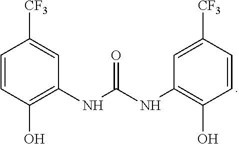 [SMILES: O=C(Nc1cc(C(F)(F)F)ccc1O)Nc1cc(C(F)(F)F)ccc1O]

Suitable potassium channel activators for use in the disclosed treatment methods may include, but are not limited to, agents that activate the KCNK3 channel. Suitable potassium channel activators may include, but are not limited to ONO-RS-082 having the following chemical structure or suitable pharmaceutical salts thereof:

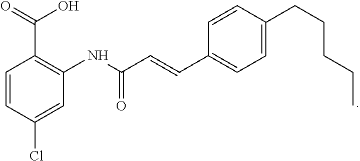 [SMILES: CCCCCc1ccc(/C=C/C(=O)Nc2cc(Cl)ccc2C(=O)O)cc1]

Suitable potassium channel activators for use in the disclosed treatment methods may include a combination of potassium channel activators. Suitable combinations of potassium channel activators may include, but are not limited to, a combination comprising ONO-RS-082 and NS1643.

Suitable potassium channel activators for use in the disclosed treatment methods may include, but are not limited to, agents that activate a BK potassium channel and/or SK potassium channel. Suitable potassium channel activators for use in the disclosed methods may include, but are not limited to, chlorzoxazone having the following chemical structure or suitable pharmaceutical salts thereof:

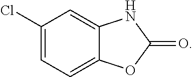 [SMILES: O=c1[nH]c2cc(Cl)ccc2o1]

Suitable therapeutic agents for use in the disclosed treatment methods may include a combination of potassium channel activators. Suitable combinations of potassium channel activators may include, but are not limited to, a combination comprising chlorzoxazone and NS1643.

Suitable therapeutic agents for use in the disclosed treatment methods may include a combination of one or more potassium channel activators and an mTOR inhibitor. The mTOR inhibitor may be administered before, concurrently with, or after administering the one or more potassium channel activators.

Suitable therapeutic agents for use in the disclosed treatment methods may include a combination of one or more potassium channel activators and rapamycin having the following formula or suitable pharmaceutical salts thereof:

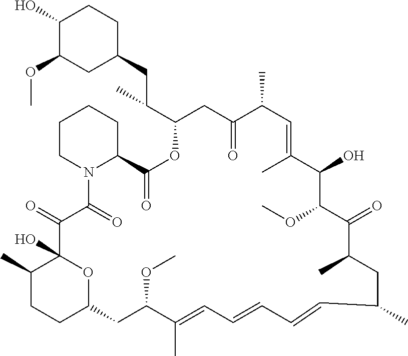

The rapamycin may be administered before, concurrently with, or after administering the one or more potassium channel activators.

Suitable therapeutic agents for use in the disclosed treatment methods may include a combination of one or more potassium channel activators and an alkylating agent. The alkylating agent may be administered before, concurrently with, or after administering the one or more potassium channel activators.

Suitable therapeutic agents for use in the disclosed treatment methods may include a combination of one or more potassium channel activators and temozolomide having the following formula or suitable pharmaceutical salts thereof:

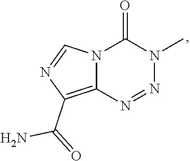

The temozolomide may be administered before, concurrently with, or after administering the one or more potassium channel activators.

Suitable therapeutic agents for use in the disclosed treatment methods may include a combination of one or more potassium channel activators and a corticosteroid, which may include but is not limited to dexamethasone, betamethasone, triamcinolone acetonide, fluorometholone, cortisone, hydrocortisone, fludrocortisone acetate, prednisolone, prednisone, methylprednisolone, triamcinolone. The corticosteroid may be administered before, concurrently with, or after administering the one or more potassium channel activators.

Suitable therapeutic agents for use in the disclosed treatment methods may include a combination of one or more potassium channel activators and dexamethasone or a derivative thereof having the following chemical structure and suitable pharmaceutical salts thereof:

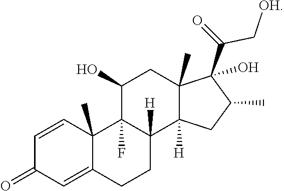

The dexamethasone or a derivative thereof may be administered before, concurrently with, or after administering the one or more potassium channel activators.

Suitable therapeutic agents for use in the disclosed treatment methods may include a combination of one or more potassium channel activators and a proton pump inhibitor, which may include but is not limited to pantoprazole, omeprazole, lansoprazole, dexlansoprazole, esomeprazole, rabeprazole, and ilaprazole. The proton pump inhibitor may be administered before, concurrently with, or after administering the one or more potassium channel activators.

Suitable therapeutic agents for use in the disclosed treatment methods may include a combination of one or more potassium channel activators and pantoprazole having the following chemical structure or suitable pharmaceutical salts thereof:

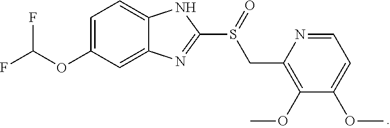 [SMILES: COc1ccnc(CS(=O)c2nc3cc(OC(F)F)ccc3[nH]2)c1OC]

The pantoprazole may be administered before, concurrently with, or after administering the one or more potassium channel activators.

Suitable therapeutic agents for use in the disclosed treatment methods may include a combination of one or more potassium channel activators and one or more sodium channel inhibitors (e.g., a sodium channel blocker). The sodium channel inhibitor may be administered before, concurrently with, or after administering the one or more potassium channel activators.

Suitable therapeutic agents for use in the disclosed treatment methods may include a combination of one or more potassium channel activators and lamotrigine having the following chemical structure or suitable pharmaceutical salts thereof:

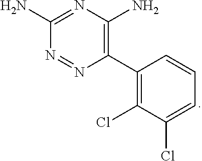 [SMILES: Nc1nnc(-c2cccc(Cl)c2Cl)c(N)n1]

The lamotrigine may be administered before, concurrently with, or after administering the one or more potassium channel activators.

Suitable therapeutic agents for use in the disclosed treatment methods may include a combination of one or more potassium channel activators and cariporide having the following chemical structure or pharmaceutical salts thereof:

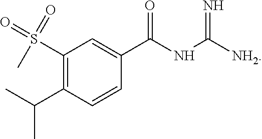 [SMILES: CC(C)c1ccc(C(=O)NC(=N)N)cc1S(C)(=O)=O]

The cariporide may be administered before, concurrently with, or after administering the one or more potassium channel activators.

Suitable therapeutic agents for use in the disclosed treatment methods may include a combination of one or more potassium channel activators and topiramate having the following chemical structure or suitable pharmaceutical salts thereof:

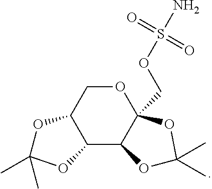

The topiramate may be administered before, concurrently with, or after administering the one or more potassium channel activators.

Suitable therapeutic agents for use in the disclosed treatment methods may include a combination of one or more potassium channel activators and one or more calcium channel inhibitors (e.g., a calcium channel blocker). The calcium channel inhibitor may be administered before, concurrently with, or after administering the one or more potassium channel activators.

Suitable therapeutic agents for use in the disclosed treatment methods may include a combination of one or more potassium channel activators and gabapentin having the following chemical structure or suitable pharmaceutical salts thereof:

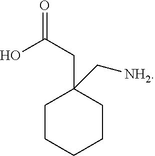 [SMILES: NCC1(CC(=O)O)CCCCC1]

The gabapentin may be administered before, concurrently with, or after administering the one or more potassium channel activators.

Suitable therapeutic agents for use in the disclosed treatment methods may include a combination of one or more potassium channel activators and zolmitriptan having the following chemical structure or suitable pharmaceutical salts thereof:

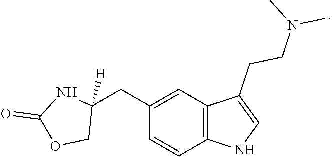 [SMILES: CN(C)CCc1c[nH]c2ccc(C[C@H]3COC(=O)N3)cc12]

The zolmitriptan maybe administered before, concurrently with, or after administering the one or more potassium channel activators.

Suitable therapeutic agents for use in the disclosed treatment methods may include a combination of one or more potassium channel activators and a peroxisome proliferator-activated receptor alpha (PPARα) activator (e.g., a PPARα agonist). The PPARα activator may be administered before, concurrently with, or after administering the one or more potassium channel activators.

Suitable therapeutic agents for use in the disclosed treatment methods may include a combination of one or more potassium channel activators and fenofibrate having the following chemical structure or suitable pharmaceutical salts thereof:

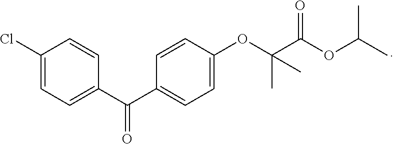 [SMILES: CC(C)OC(=O)C(C)(C)Oc1ccc(C(=O)c2ccc(Cl)cc2)cc1]

The fenofibrate may be administered before, concurrently with, or after administering the one or more potassium channel activators.

In some embodiments, the disclosed subject matter relates to methods for treating a cell proliferative disease or disorder in a subject in need thereof. The treatment methods may include administering to the subject: (i) an effective amount of a sodium channel inhibitor (e.g., a sodium channel blocker); and (ii) an effective amount of a mTOR inhibitor, where the sodium channel inhibitor is administered to the subject before, concurrently with, or after the mTOR inhibitor is administered to the subject.

Suitable sodium channel inhibitors for use in the disclosed treatment methods may include, but are not limited to, agents that inhibit a voltage-gated sodium channel. Suitable sodium channel inhibitors may include but are not limited to lamotrigine and topiramate.

Suitable sodium channel inhibitors for use in the disclosed treatment methods may include, but are not limited to, agents that inhibit Na(+)/H(+) exchanger type 1 (NHE1). Suitable sodium channel inhibitors may include but are not limited to cariporide.

Suitable therapeutic agents for use in the disclosed methods include agents that inhibitor mTOR. Suitable mTOR inhibitors may include but are not limited to rapamycin.

In some embodiments, the disclosed subject matter relates to methods for treating a cell proliferative disease or disorder in a subject in need thereof. The treatment methods may include administering to the subject: (i) an effective amount of a sodium channel inhibitor (e.g., a sodium channel blocker); and (ii) an effective amount of a proton pump inhibitor, where the sodium channel inhibitor is administered to the subject before, concurrently with, or after the proton pump inhibitor is administered to the subject.

Suitable sodium channel inhibitors for use in the disclosed treatment methods may include, but are not limited to, agents that inhibit a voltage-gated sodium channel. Suitable sodium channel inhibitors may include but are not limited to lamotrigine and topiramate.

Suitable sodium channel inhibitors for use in the disclosed treatment methods may include, but are not limited to, agents that inhibit Na(+)/H(+) exchanger type 1 (NHE1). Suitable sodium channel inhibitors may include but are not limited to cariporide.

Suitable therapeutic agents for use in the disclosed methods include agents that are proton pump inhibitors. Suitable proton pump inhibitors may include but are not limited to pantoprazole.

In some embodiments, the disclosed subject matter relates to methods for treating a cell proliferative disease or disorder in a subject in need thereof. The treatment methods may include administering to the subject: (i) an effective amount of a calcium channel inhibitor (e.g., a calcium channel blocker); and (ii) an effective amount of a mTOR inhibitor, wherein the calcium channel inhibitor is administered to the subject before, concurrently with, or after the mTOR inhibitor is administered to the subject.

Suitable calcium channel inhibitors for use in the disclosed treatment methods may include, but are not limited to, agents that inhibit a voltage-gated calcium channel. Suitable calcium channel inhibitors may include but are not limited to zolmitriptan.

Suitable therapeutic agents for use in the disclosed methods include agents that inhibitor mTOR. Suitable mTOR inhibitors may include but are not limited to rapamycin.

In some embodiments, the disclosed subject matter relates to methods for treating a cell proliferative disease or disorder in a subject in need thereof. The treatment methods may include administering to the subject: (i) an effective amount of a proton pump inhibitor; and (ii) an effective amount of an alkylating agent, where the proton pump inhibitor is administered to the subject before, concurrently with, or after the alkylating agent is administered to the subject. Suitable proton pump inhibitors may include, but are not limited to, pantoprazole. Suitable alkylating agents may include, but are not limited to, temozolomide.

Suitable cell proliferative diseases and disorders treated by the disclosed methods may include cancers, for example, cancers that express a potassium channel (e.g., a KCNQ/Kv7 channel, a K(ATP) channel, a hERG channel, or a KCNK3 channel), a sodium channel (e.g. a voltage-gated sodium channel or a sodium/proton exchanger such as NHE1), or a calcium channel (e.g., a voltage-gated calcium channel). Cancer treated by the disclosed methods may include, but are not limited to, brain cancer (e.g., glioblastoma multiforme (GBM)), prostate cancer, breast cancer, lung cancer (e.g., non-small cell lung cancer (NSCLC)), colorectal cancer, bladder cancer, kidney cancer, uterine cancer, melanoma, lymphoma (e.g., Non-Hodgkin lymphoma), leukemia, pancreatic cancer, ovarian cancer, liver and intrahepatic bile duct cancer, oral cavity cancer, and esophageal cancer.

The compounds utilized in the methods disclosed herein may be formulated as anti-cancer therapeutics, including therapeutics for malignancies including cancers such as brain cancer (e.g., glioblastoma multiforme (GBM)), prostate cancer, breast cancer, lung cancer (e.g., non-small cell lung cancer (NSCLC)), colorectal cancer, bladder cancer, kidney cancer, uterine cancer, melanoma, lymphoma (e.g., Non-Hodgkin lymphoma), leukemia, pancreatic cancer, ovarian cancer, liver and intrahepatic bile duct cancer, oral cavity cancer, and esophageal cancer.

The compounds utilized in the methods disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.01 μM, 0.5 μM, 0.1 μM, 1.0 μM, 10 μM, 100 μM, 200 μM, 400 μM, 800 μM, and 1000 μM (e.g., 1.0 μM-800 μM).

The compounds utilized in the methods disclosed herein and pharmaceutical compositions comprising the compounds may be administered in methods of treating a subject in need thereof. For example, in the methods of treatment a subject in need thereof may include a subject having a cell proliferative disease, disorder, or condition such as cancer (e.g., cancers such as brain cancer (e.g., glioblastoma multiforme (GBM)), prostate cancer, breast cancer, lung cancer (e.g., non-small cell lung cancer (NSCLC)), colorectal cancer, bladder cancer, kidney cancer, uterine cancer, melanoma, lymphoma (e.g., Non-Hodgkin lymphoma), leukemia, pancreatic cancer, ovarian cancer, liver and intrahepatic bile duct cancer, oral cavity cancer, and esophageal cancer).

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as endpoints any of these disclosed doses (e.g., 2.5-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as end-points any of these disclosed dose levels (e.g., 500-2000 ng/kg body weight of the subject).

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel®. PH101 and Avicel® PH102; lactose such as lactose monohydrate, lactose anhydrous, and Pharmatose® DCL21; dibasic calcium phosphate such as Emcompress.®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules. In some embodiments, the compounds are formulated as a composition for administration orally (e.g., in a solvent such as 5% DMSO in oil such as vegetable oil).

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

### EXAMPLES

The followings Examples are illustrative only should not be interpreted to limit the scope of the claimed subject matter.

### Example 1—Use of Bioelectric Drugs for Suppressing Cancer Cell Behaviors

The present inventors have determined that various hyperpolarizing and serotonergic drugs may be utilized alone or in combination with rapamycin and other small drug-like molecules in order to suppress growth and differentiation of glioblastoma cells in vitro. The inventors examined several known bioelectric drugs (i.e., candidate ionoceuticals)—alone, in combinations, and in combinations with established drug therapies, for the ability to reduce proliferation of mammalian glioblastoma cell lines in vitro.

The inventors studied hyperpolarizing drugs including retigabine, minoxidil, NS1643, lamotrigine, and zolmitriptan. The inventors tested these compounds either alone or in combination with other drugs including temozolomide (TMZ), pantoprazole, and rapamycin.

cAMP has been used in combination with rapamycin to differentiate and arrest the cell cycle of mouse-rat hybrid NG108-15 cells (glioblastoma and neuroblastoma). Rapamycin is known to induce an autophagic response while cAMP hyperpolarizes the cells.

Experiments herein show that combinations of drugs drive the differentiation and cell cycle arrest of NG108-15 glioblastoma stem cells in vitro. NG108-15 cells were transfected with a fluorescent cell cycle reporter and fluorescent marker for the cell membrane. Cells were plated in 96 well plates at a low confluence and drugs were added fresh every other day for 6 days. Some studies removed the drug at 6 days and added media with no drug for an additional 4 days to test for proliferation. Images of the cells were taken every day and a cell profiler pipeline was used to count the cells and determine the ratio of cells in each part of the cell cycle. Rapamycin at 100 nM, 150 nM, and 200 nM showed a very significant decrease in proliferation and an increase in cells arrested at G1, confirming what other studies have found in human derived glioblastoma cells. Pantoprazole at 100 μM also showed a very significant decrease in cell proliferation and an increase in cells arrested at late S phase, also confirming what other studies have found in human derived glioblastoma cells. Temozolomide at 50 μM in combination with these two compounds was used as a comparison for the efficacy of novel combinations because temozolomide is currently the leading drug in the treatment of glioblastoma. Combinations of zolmitriptan at 10 μM, or retigabine at 10 μM, or lamotrigine at 100 μM, or minoxidil at 30 μM with rapamycin at 200 nM showed significant decreases in cell proliferation as compared to rapamycin alone. Combinations of lamotrigine at 100 μM, or retigabine at 10 μM with pantoprazole at 100 μM also showed significant decreases in cell proliferation as compared to pantoprazole alone. Retigabine in combination with pantoprazole also showed a very significant increase in the number of cells arrested at late S phase with recovery of cell proliferation.

### Example 2—Ion Channel Drugs Suppress Cancer Phenotype in NG108-15 Cells: Toward Novel Electroneuticals for Glioblastoma

Glioblastoma is a highly lethal brain cancer that commonly recurs after tumor resection and chemotherapy treatment. Depolarized resting membrane potentials and an acidic intertumoral extracellular pH have been associated with a proliferative state and drug resistance, suggesting that forced hyperpolarization and disruption of proton pumps in the plasma membrane could be a successful strategy for targeting glioblastoma overgrowth. Screening of 139 compounds, concentrations, and combinations of ion-modulating drugs was conducted in the NG108-15 rodent neuroblastoma/glioma cell line. A subset of these were then tested in the U87 human glioblastoma cell line. A FUCCI cell cycle reporter was stably integrated into both cell lines and live imaging monitored the effects on proliferation and cell cycle response. Immunocytochemistry, electrophysiology, and a panel of physiological dyes reporting voltage and pH were used to characterize responses. The most effective treatments on proliferation in U87 cells were combinations of NS1643 and pantoprazole; retigabine and pantoprazole; and pantoprazole or NS1643 with temozolomide. Marker analysis and physiological dye signatures suggest that exposure to bioelectric drugs significantly reduces proliferation, makes the cells senescent, and promotes differentiation. These results, along with the observed low toxicity in human neurons, show the high efficacy of electroceuticals utilizing combinations of repurposed FDA approved drugs.

Alongside canonical biochemical factors and biomechanical forces, cell and tissue-level order are now known to be regulated by bioelectrical signaling among many cell types [11,20]. The transmembrane voltage (Vₘₑₘ) of cells is regulated by ion channels, which are not only important targets in embryonic channelopathies [21-23] but also increasingly seen as cancer targets [21,24-35]. Overall, adult non-proliferative cells tend to have hyperpolarized membrane potentials whereas stem cells, embryonic cells, and other highly proliferative cells are much more depolarized [21,36]. Cancer cell Vₘₑₘ tends to be more similar to that of embryonic cells, with much more depolarized membrane potentials than their non-cancerous equivalents [36,37]. Indeed, transformed cells can be detected in vivo in animal models using voltage-sensitive fluorescent dyes based on their abnormal bioelectric signature [38]. Importantly, resting membrane potential is not only a marker, but is functionally instructive for cell behavior. Classic work by Cone showed that Vₘₑₘ is an important regulator of proliferation in terminally differentiated cells or cancer [39-41] and this has been confirmed in recent work linking depolarization with a plastic, undifferentiated, highly proliferative state. For example, treatment of mesenchymal stem cells with depolarizing drugs inhibited their differentiation into adipocytes or osteoblasts, suggesting that depolarized Vₘₑₘ is not only correlated with stemness, but that hyperpolarization is required for differentiation[42-46]. Importantly, in vivo experiments showed that co-expression of hyperpolarizing ion channels, or optogenetic activation of channels [47] in the context of human oncogenes expressed in Xenopus tadpoles, can prevent the tumors that form when oncogenes are expressed alone [33,38,48]. In mammalian models, several recent studies have shown that ion channelopathies are present in many cancers and play a key role in cell proliferation, progression through the cell cycle, and metastasis [24,28,30,35,49-55].

The location of many ion channels in the outer cell membrane also makes them attractive targets. Agonists or antagonists used to modulate Vₘₑₘ can be chosen for binding sites on the outer side of the membrane, bypassing the challenges posed by increased drug efflux transporters in glioblastoma stem cells (GSCs). Furthermore, ion channel transcripts have been shown to be upregulated in GSCs, including SCN8A which encodes a sodium channel, KCNB1 which encodes a voltage-gated potassium channel, and GRIA3 which encodes an ionotropic glutamate receptor that is non-selective for monovalent cations [53]. In addition, intracellular alkalization induced by dysfunction in proton transportation has been shown to increase drug resistance in glioblastoma (GBM) and contributes to extracellular acidification, which provides GSCs with an optimized niche and facilitates the release of large oncosomes, a type of extracellular vesicle which can transform neighboring cells [56-58]. Thus, due to the location and importance of these channels in cancer cell proliferation, migration, and metastasis, ion channels provide an optimal target for development of new GBM treatments.

To discover effective interventions for glioblastoma among already-known small molecule modulators of bioelectric state, the NG108-15 cell-line was first used to screen for Vₘₑₘ-modifying compounds that could potentially promote differentiation in glioblastoma cells [59]. NG108-15 cells are a hybrid formed from mouse N18TG2 neuroblastoma cells with rat C6-BU-1 glioma cells and is a popular model system in neuronal differentiation studies. This line was chosen due to its extensive use in determining the factors necessary for neuronal differentiation, its close resemblance to NSCs and GSCs, and its well characterized membrane properties and electrophysiology [60-62]. The goal was to find compounds that would safely abrogate the proliferative potential in NG108-15 cells under high serum conditions, normally prohibitive for differentiation, revealing robust candidates for subsequent assessment in the U87 (ATCC) human glioblastoma line, and for future testing in patient derived GSCs under serum-free conditions [63-66].

To screen candidate drugs targeting ion channels and pumps, and their combinations, new tools were created: a NG108-15 and U87 stable line expressing a fluorescent FUCCI cell cycle indicator [67] and a cell membrane mTurquoise tag for revealing whole cell morphology. Automated electrophysiology was used on NG108-15 cells to determine what the combinations did to overall transmembrane potential. For U87 cells, dyes were used to determine cytoplasmic calcium levels, internal pH (pHi), transmembrane potential, lysosomal pH, and perform a Live/Dead assay to gain a comprehensive profiling of the drugs' effects on cell physiology. Finally, a beta-galactosidase stain and immunocytochemistry were used to analyze senescence, cell cycle inhibitor levels, and differentiation status of treated NG108-15 and U87 cells. Several combinations of compounds were identified that appear to facilitate a reduction of proliferation and an induction of differentiation, demonstrating that this class of electroceuticals, most of which are already FDA approved for other conditions, are good candidates for cancer remediation in GBM.

### Methods

Reagents. Stocks of Rapamycin (R8781, Sigma), Retigabine (SML0325, Sigma), Minoxidil (M4145, Sigma), NS1643 (sc-2041353, Santa Cruz Biotechnology), Lamotrigine (L3791, Sigma), Zolmitriptan (SML0248, Sigma), Cariporide (SML1360, Sigma), Topiramate (T0575, Sigma), Pantoprazole Sodium Hydrate (P0021, Sigma), Fenofibrate (F6020, Sigma), Acetazolamide (A6011, Sigma), Quercetin (Q4951, Sigma), Temozolomide (2706, Tocris), Dexamethasone (D4902, Sigma), ONO-RS-082 (00766, Sigma), Topotecan hydrochloride (4562, Tocris), CKD 602 (5125, Tocris), (Z)-4-hydroxytamoxifen (3412, Tocris), Lansoprazole (2582, Tocris), Chlorzoxazone (C4397, Sigma), and Sodium Butyrate (B5887, Sigma), were made at 1000× concentration in DMSO. Stocks of Gabapentin (G154, Sigma) and Cisplatin (232120, EMD Millipore) were made in water at 1000×. Dibutyryl cAMP sodium salt (D0627, Sigma) was the only compound dissolved directly in cell culture media at 1 mM concentration.

Cell Culture. NG108-15 cells (ATCC), passage 7-10 were cultured in growth media containing DMEM medium with high glucose and no phenol red or sodium pyruvate (31053-028, ThermoFisher), which was supplemented with 2 mM Glutamax, 10% FBS, HAT supplement, and 10 U/mL penicillin/streptomycin. Cells were disassociated with Accutase and passaged when 70% confluent and maintained at 37° C. with 5% C02. Media was changed every two days. For live cell assays, plates were coated with 1/50 dilution of no phenol red growth factor reduced (GFR) Matrigel (356231, Corning) in imaging media containing Fluorobrite DMEM (A1896701, ThermoFisher) with no added supplements. Cell culture media was switched to Fluorobrite DMEM with 2 mM Glutamax, 10% FBS, HAT supplement, and 10 U/mL penicillin/streptomycin for all live cell imaging experiments, except for a cAMP with rapamycin treatment which was done in the exact same media except with 1% FBS. All drug screens including controls were done using 0.02% DMSO in the high serum Fluorobrite DMEM.

U87 (ATCC) cells were cultured under the same conditions as the NG108-15 cells minus the HAT supplement and used at passage 7-10 for experiments. All cells plated for antibody staining and senescence assays were cultured on polyethyleneimine (181978, Sigma) coated plates at 25 μg/mL in 150 mM NaCl solution for 1 hour at room temperature followed by a rinse of PBS and an additional coating of 1/50 Matrigel as described above.

Human neuronal cells were differentiated from human induced neural stem cells (hiNSCs) (passage 7-10), a generous gift from David Kaplan, as described previously [68]. Briefly, hiNSCs were grown on mouse embryonic fibroblast (MEF) feeder cells until ready to differentiate into neurons. Cells were then seeded on Poly-D-Lysine (A3890401, ThermoFisher) and laminin (L2020, Sigma) coated 96 well plates at a density of 128,000 cells/mL. Cells were differentiated in Neurobasal (12348017, ThermoFisher) media supplemented with 2% B27 (17504044, Gibco), 1% Glutamax, and 1% antibiotic-antimycotic for 7 days with media changes every 2 days. At the end of the 7 days the treatments were added in the differentiation media for 3 days.

Molecular Biology. The ES-FUCCI construct containing a hygromycin resistance cassette was subcloned using XmnI and SalI from the plasmid ES-FUCCI, which was a gift from Pierre Neveu (Addgene plasmid #62451; http://n2t.net/addgene:62451; RRID:Addgene_62451) [67]. The CAG pPalmitoyl-mTurquoise2 construct was subcloned from the plasmid pPalmitoyl-mTurquoise2 using BamHI and NotI, which was a gift from Dorus Gadella (Addgene plasmid #36209; http://n2t.net/addgene:36209; RRID:Addgene_36209) [69]. All subcloned fragments were cloned into a pENTR1A plasmid with a CAG promoter and multiple cloning site (MCS) followed by a SV40 poly(A) using the same sites as were used in excising the fragment from the parent plasmid. In the case of ES-FUCCI the CAG promoter, MCS, and poly(A) were removed from the pENTR1A plasmid with SpeI, which was blunted, and SalI prior to ligation with the fragment. The resulting pENTR1A ES-FUCCI was then Gateway LR clonased (11791020, ThermoFisher) into the hyperactive piggyBac transposase-based, helper-independent, and self-inactivating delivery system, pmhyGENIE-3, a gift from Stefan Moisyad [70,71]. The pENTR1A CAG pPalmitoyl-mTurquoise2 was cloned into a pmhyGENIE-3 containing a neomycin resistance gene in the backbone. The resulting plasmids, HypG3 Hygro ES-FUCCI, and HypG3 NeoBB CAG pPalmitoyl-mTurquoise2 were used for subsequent transfections.

Generation of Stable Lines. All transgenic cell lines were made by transfecting cells at 30% confluence with 500 ng of appropriate HypG3 plasmid via 1 μL of lipofectamine 3000 (L3000008, ThermoFisher) per well of 24 well plate containing 500 μL of culture media. Reagent was removed after 24 hours and fresh culture media was added. Cells were allowed to recover for 24 hours prior to selection with 1000 μg/mL G418 or 200 μg/mL hygromycin. After selection, cells were serially diluted into 96 well plates and single colony clones were expanded. Clones showing robust growth and strong expression were chosen for subsequent experiments.

Growth and FUCCI assays. Both cell lines were plated at 5,000 cells per mL into black-walled flat-bottom 96-well plates coated with 1/50 dilution of Matrigel. Images of cells were taken on Day 0 and every subsequent day for 10 days using a Zeiss Axio1 fitted with an on-stage incubator and kept at 37° C. with 5% C02. A 5× objective was used along with filters for YFP, RFP, and CFP and careful calibration of each plate was done to ensure that the same fields of view were imaged each day. Drugs were added after the initial images were taken and then changed every two days until day 6. For recovery experiments, after imaging on day 6, all drugs were removed, and cells were put into imaging media without any added drugs. This media was changed every two days until day 10. Analysis of growth and snap shots of FUCCI reporters over the 6 days or 10 days was done using a CellProfiler [72]pipeline designed by the Broad. The fractional difference in cell number was calculated by dividing the total number of cells each day by the total number of cells from day 0. The ratio of cells in each cell cycle stage every day was determined by counting all the nuclei of one color and dividing it by the total number of nuclei. Blind quality control was done on all the images to make sure that any fibers or image artifacts were not incorrectly counted by the program.

Antibody Staining, BrdU and Senescence Assays. NG108-15 cells were plated on PEI and Matrigel coated plates at 15,000 or 30,000 cells/mL, U87 cells at 10,000 cells/mL and grown in imaging media for 6 days with drug treatment. Fresh media was added every 2 days. On day 6, cells were fixed with 4% formaldehyde in PBS for 30 minutes for all antibody staining, except for BrdU which was fixed for 15 minutes, and for the senescence assay which was fixed for 20 min using the Senescence Beta-Galactosidase Staining Kit (9860, CellSignaling) and thereafter stained according to manufacturer's protocol. After fixing, cells were washed twice with PBS, and were permeabilized in PBS with 0.3% Triton-X for 15 min then washed again twice with PBS. The cells for the BrdU assay were additionally treated with 1N HCl for 10 min on ice, followed by a treatment with 2N HCl for 50 min at room temp. Cells were then blocked with standard blocking buffer comprised of TBS containing 10% goat serum, 0.1% BSA, and with 0.05% Tween 20 or without detergent for antibodies against phosphorylated proteins or monoclonal antibodies except for Anti-YAP and Anti-BrdU staining, which were done according to manufacturer's protocol. Primary antibodies were added at the following concentrations: 1:250 anti-S100 beta (GTX129573, Genetex), 1:500 anti-GFAP (AB5804, EMD-Millipore), 1:300 anti-SOX10 (ab155279, Abcam), 1:100 MAP2 (4542, CellSignaling), 1:400 anti-cleaved caspase 3 (9661, CellSignaling), 1:300 anti-Cx43 (STJ2411, St. John's Labratory), 1:800 anti-p27^{Kip1} (3698, CellSignaling), 1:150 anti-BrdU (5292, CellSignaling), 1:125 anti-LC3-II (2775, CellSignaling), 8 μg/mL anti-04 (MAB1326, R&D Systems), 1:1000 anti-TH (RPCA-TH, Encor), 1:4000 anti-NSE (RPCA-NSE, Encor), 1:4000 anti-NFM (RPCA-NF-M, Encor), 0.5 μg/mL anti-TujI (801202, Biolegend), 1:400 anti-NF-KB p65 (8242, CellSignaling), 1:125 anti-phospho CREB (05-807, EMD-Millipore), 1:250 anti-vimentin (ab92547, Abcam), 1:125 anti-YAP (14074, CellSignaling). Antibodies were diluted in their respective blocking buffer and incubated overnight at 4° C. The next day, cells were washed with TBS-T or TBS for 3× for 5 min each and secondary antibody was added as follows: 1:1000 donkey anti-mouse 647 (A-31571, Thermo-Fisher) or 1:1000 donkey anti-rabbit 647 (A-31573, Thermo-Fisher). Each were diluted in blocking buffer along with 2.5 μg/mL Hoechst 33342 and incubated for 1 hour at room temperature. Cells were then washed again with TBS-T or TBS for 3× for 5 min each and covered with Gelvatol. Cells were imaged with an EVOS M7000 system, with at least 50% of each well in a 96 well plate imaged and analyzed using a CellProfiler pipeline for measuring integrated intensity, mean intensity, or nuclear to cytoplasmic ratio of mean intensities [73].

Dye Staining Protocols. For the Live/Dead assay, 1 μM Calcein Green AM and 0.5 μM ethidium homodimer-1 (L3224, ThermoFisher) were added to PBS along with 10 μg/mL of Hoechst to make the staining solution. Half the media was removed from the human neuronal cells that had been incubated with treatments for 3 days and replaced with staining solution. This was done four times to make sure that cells did not detach. Cells were then incubated for 15 min at 37° C. and imaged. For resting membrane potential staining, DiBAC4(3) was used. U87 cells were seeded at 10,000 cells per mL on Matrigel coated plates as described above. Cells were treated with drugs for 6 days with changes in media every other day. On day 6 the media was removed and washed once with dye buffer consisting of Hank's Balanced Salt Solution (HBSS) with 20 mM HEPES at pH 7.4 and then the staining solution, consisting of dye buffer with 2 μM DiBAC4(3) was added to the cells and allowed to incubate for 30 min at 37° C. The staining solution was then removed and fresh staining solution containing the treatments were added to the cells. For cytoplasmic calcium staining, Fluo-4 AM was used. The staining solution consisted of dye buffer with 4 μM Fluo-4 AM and a 1:1 ratio of Pluronic F-127 (20% in DMSO) and allowed to incubate for 30 min at room temperature. The staining solution was then removed, and fresh dye buffer was added and allowed to incubate for 20 min at 37° C. Then dye buffer containing the treatments were added to the cells. For cytoplasmic pH staining, pHrodo Green was used. The staining solution consisted of dye buffer with a 1:1000 dilution of the pHrodo Green stock and a 1:100 dilution of the PowerLoad concentrate (P35373, ThermoFisher). The cells were allowed to incubate for 30 min at 37° C. Staining solution was removed and washed once with dye buffer then fresh dye buffer containing the treatments were added and allowed to incubate for 5 min at 37° C. Calibration curve was done by instead adding the components of the intracellular pH calibration kit (P35379 ThermoFisher). For lysosomal pH staining, LysoSensor Green was used. The staining solution consisted of dye buffer with a 1 μM dilution of LysoSensor Green DND-187 (L7535 ThermoFisher). The cells were incubated as above and staining solution was removed and replaced with dye buffer containing 2.5 μg/mL Hoechst 33342 for 10 min. Hoechst was removed and cells were washed with dye buffer one time before adding dye buffer containing the treatments. Cells were imaged using an EVOS M7000 system outfitted with a GFP filter cube for stains and a DAPI filter for Hoechst. Images were then analyzed using a CellProfiler pipeline.

Electrophysiology. SyncroPatch 384PE platform (Nanion Technologies®) was used to perform the automated patch-clamp experiments. This system provides giga-ohm resistance seals during simultaneous recording in the 384-well plate format. Cells were harvested 48-72 h after seeding 1-2×10⁶ cells in T175 culture flasks (Falcon), then cells were rinsed with PBS (5 mL) and treated with 3 mL Accutase (STEMCELL technologies) for 5 min at 37° C., re-suspended in 10 mL of serum-free media and pelleted at 1000 rpm for 3 min at RT. The supernatant was discarded, and cells are re-suspended in serum-free DMEM medium with high glucose and no phenol red or sodium pyruvate (31053-028, ThermoFisher), and physiological extracellular solution (pECS) 50% (v:v). The cells were kept until the moment of the experiment in a temperature controlled dedicated reservoir at 10° C. and shaken at 200 rpm as described [74]. The experiments were performed within one hour after the harvesting process. The assays were carried in single-hole chips with resistances between 4-5 MΩ after priming the chip with the following solutions (in mM), physiological extracellular solution (pECS) 10 HEPES, 140 NaCl, 5 Glucose, 4 KCl, 2 CaCl₂), 1 MgCl₂, 295-305 mOsm pH 7.4 (NaOH). Internal recording solution (in mM) 20 EGTA, 50 KCl, 10 NaCl, 60 KF, 10 HEPES at pH 7.2, and 285 mOsm. 15 μL of the cell suspension (50% v/v pECS/Medium no serum) was added to each well to a final density of 50-80K cells/mL. Cell capture was promoted by holding a negative pressure of −100 mbar for 20 s. After successive hyperpolarization steps from −30 mV to −100 mV enhanced the capture the seal followed by the transient addition of a high Ca2+ extracellular solution (80 mM NaCl, 3 mM KCl, 35 mM CaCl₂), 10 mM MgCl2, 10 mM HEPES at pH 7.4 and 298 mOsm. High Ca2+ solution is washed out by successive external exchanges replacing half of the volume of the well each time with the external recording solution. All recording solutions were prepared with ultrapure MilliQ water (18 MD-cm). After the formation of the Giga-seal, a 250 mbar pressure was applied to break the membrane patch. Once in whole-cell configuration, different voltage protocols were applied to dissect different conductances in NG108-15 cells. Signals were recorded in the current-clamp mode “I=0” (passive conditions) where no current was injected, to monitor the membrane potential. The membrane potentials were recorded for seven sweeps of 1 sec, with an inter-sweep time of 10 s. The average membrane potentials of the 7 sweeps were reported.

### Results

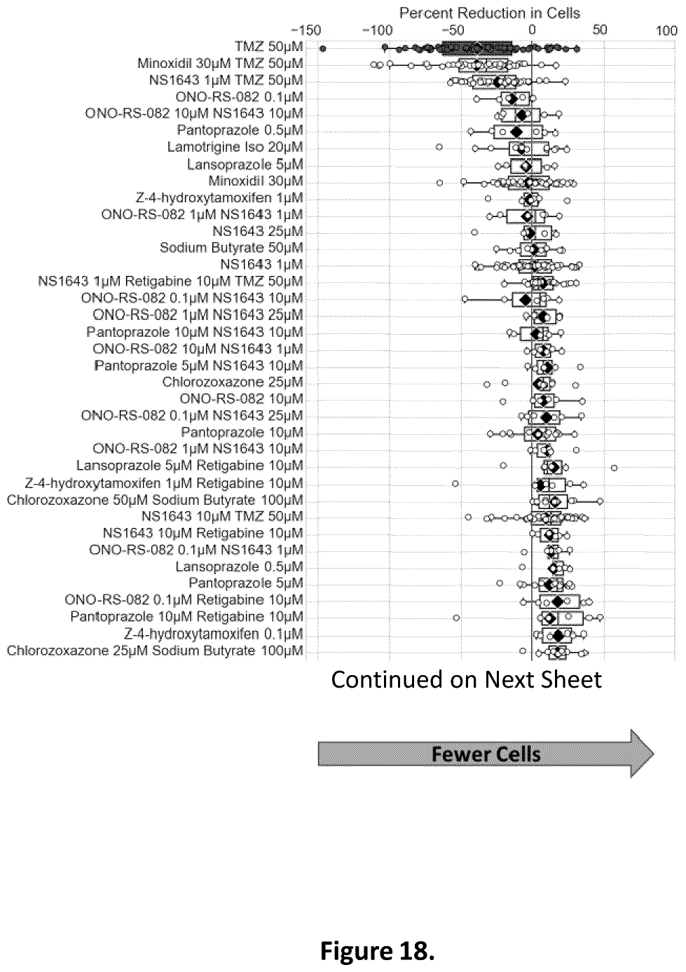

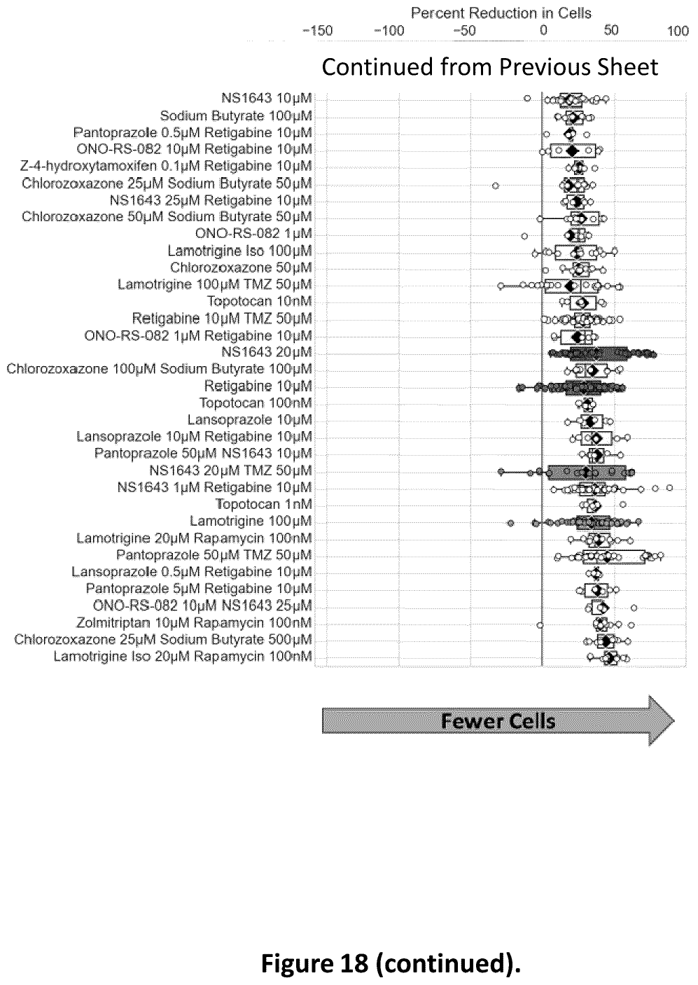

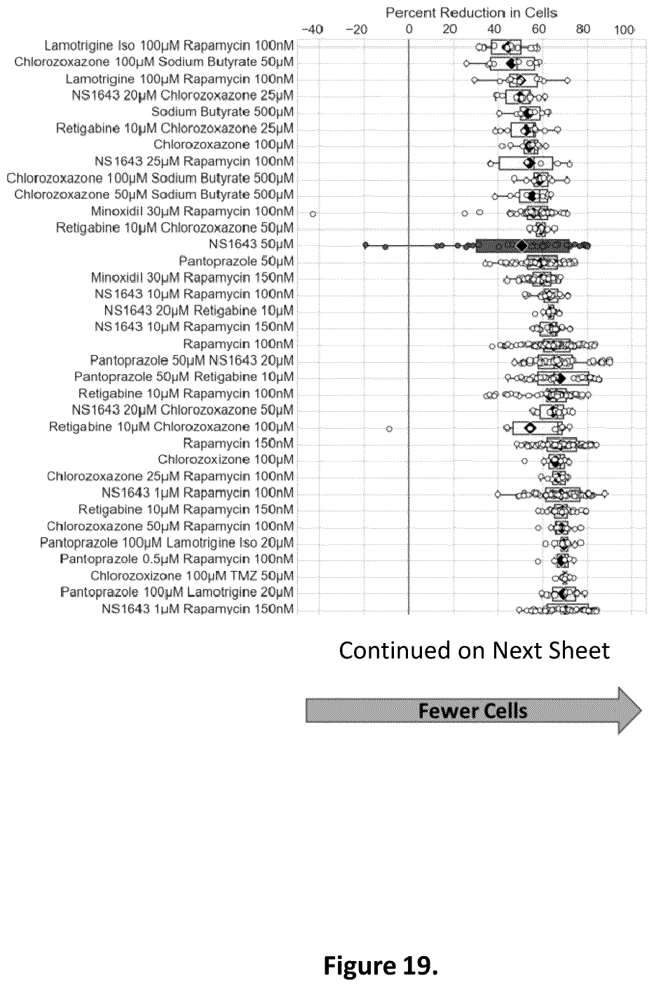

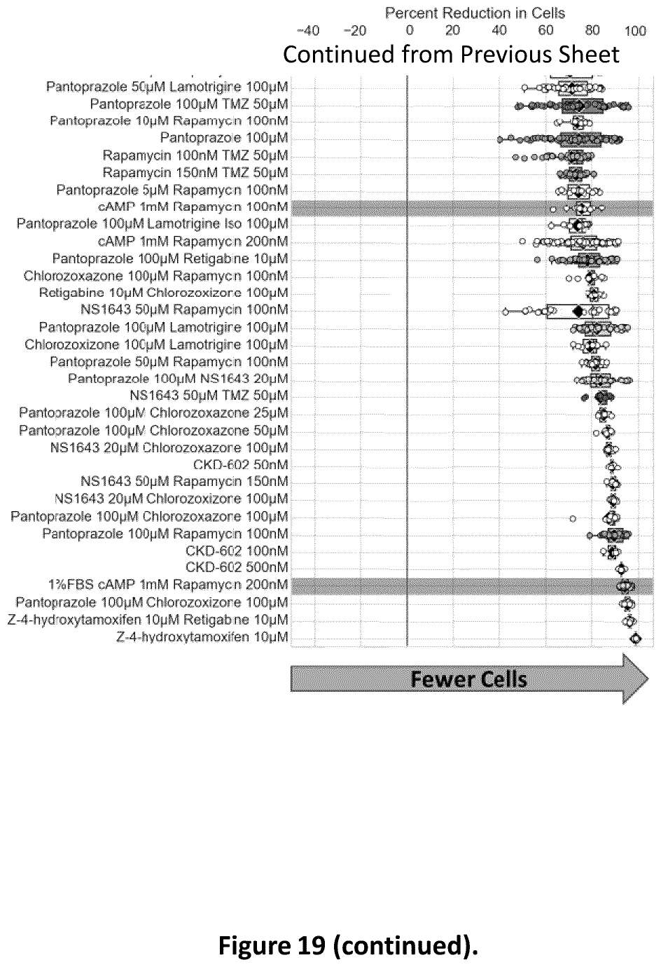

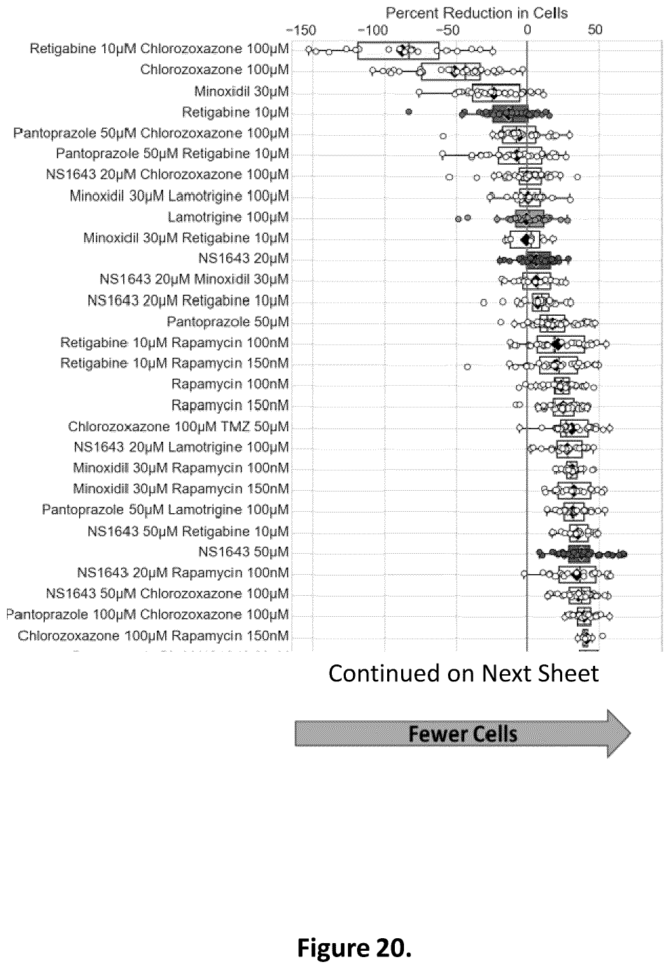

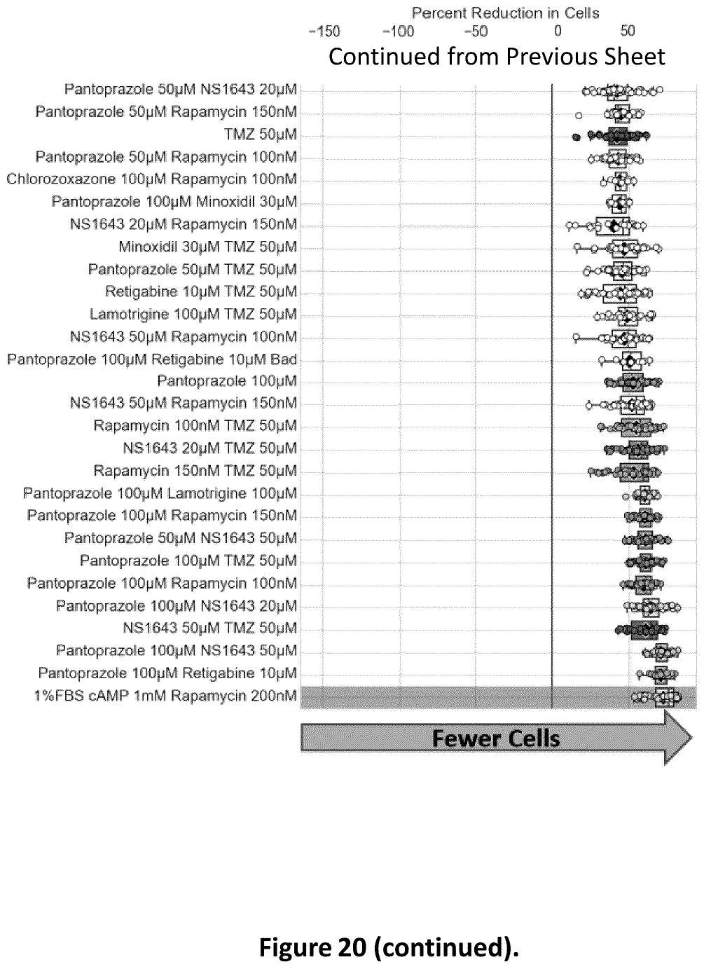

Bioelectric compounds and combinations with the proton pump inhibitor, pantoprazole stop proliferation of NG108-15 cells in high serum and shift pronortion of cells in Late S. G2, M. NG108-15 cells containing the FUCCI cell cycle reporter and palmitoyl-mTurquoise2 fluorescent membrane tag were incubated with compounds by themselves and in combination, in media containing high serum (known to be prohibitive for differentiation in this line), to test for their ability to suppress proliferation (FIG. 18-19). The compounds chosen for testing included compounds known to alter the membrane potential of the cell by decreasing proton efflux, increasing potassium efflux, or decreasing sodium influx, and others chosen for their potential combinatorial effects with the ion modulating drugs, including cell cycle specific disruptors and autophagy-inducing compounds (Table 1). In addition, the most clinically relevant compound currently used in the standard treatment of gioblastoma, temozolomide, was tested. Table 1 shows a list of the most effective compounds tested in experiments using the mouse/rat neuroblastoma/glioma NG108-15 cells containing a FUCCI cell cycle reporter and palmitoyl-mTurquise2 fluorescent membrane tag, human glioblastoma U87 cells containing the FUCCI cell cycle reporter and palmitoyl-mTurquoise2 fluorescent membrane tag, and/or induced neural human stem cells

TABLE 1
Compounds with Highest Efficacy on Proliferation
and their Potential Mechanism of Action

| Compound | Function |
|---|---|
| cAMP | Activates a variety of ion channels and protein kinases |
| Rapamycin | Inhibits mTOR and induces autophagy |
| Retigabine | Opens KCNQ/Kv7 channels |
| Minoxidil | Opens K(ATP) channels |
| NS1643 | Opens hERG channels at low concentrations |
| Gabapentin | Inhibits voltage-gated calcium channels and reduces |
| | HCN4 currents |
| Lamotrigine | Blocks voltage gated sodium channels |
| Zolmitriptan | 5-HT1B/D receptor agonist and inhibits high voltage activated |
| | Ca²⁺ channels |
| Cariporide | NHE1 inhibitor and acidifies internal pH |
| Topiramate | Blocks voltage gated sodium and calcium channels and acidifies |
| | internal pH |
| Pantoprazole | Proton pump inhibitor |
| Fenofibrate | Agonist of the PPARα, depletes ATP, and induces autophagy |
| Acetazolamide | Carbonic anhydrase inhibitor that acidifies internal pH |
| Quercetin | Flavonoid that induces autophagy |
| Temozolomide | Alkylates/methylates DNA, induces autophagy, |
| (TMZ) | used in GBM treatment |
| Dexamethasone | Corticosteroid, induces autophagy, used in GBM treatment |
| (DEX) | |
| ONO-RS-082 | Potassium two pore domain channel (KCNK3) activator |
| Topotecan | Topoisomerase I Inhibitor |
| CKD602 | Topoisomerase I inhibitor |
| z-4-hydroxytamoxifen | Blocks voltage gated sodium channels and induces autophagy |
| Lansoprazole | Proton pump inhibitor |
| Monensin | Sodium ionophore |
| Cisplatin | Binds to purine residues causes DNA damage and cell death |
| Chlorzoxazone | Increases activity of large conductance calcium activated |
| | potassium (BK(Ca)) channels |
| Sodium Butyrate | Histone deacetylase inhibitor |

A subset of the compounds exhibiting the highest efficacy in the initial screens are shown in Table 2.

TABLE 2
Compounds Tested on NG108-15 Cells for Effect on
Proliferation and their Potential Mechanism of Action

| Compound | Function |
|---|---|
| cAMP | Activates a variety of ion channels and protein kinases |
| Rapamycin | Inhibits mTOR and induces autophagy |
| Retigabine | Opens KCNQ/Kv7 channels |
| Minoxidil | Opens K(ATP) channels |
| NS1643 | Opens hERG channels at low concentrations |
| Lamotrigine | Blocks voltage gated sodium channels |
| Pantoprazole | Proton pump inhibitor |
| Temozolomide | Alkylates/methylates DNA, induces autophagy |
| (TMZ) | |
| Chlorozoxazone | SK and BK-type potassium channel activator |

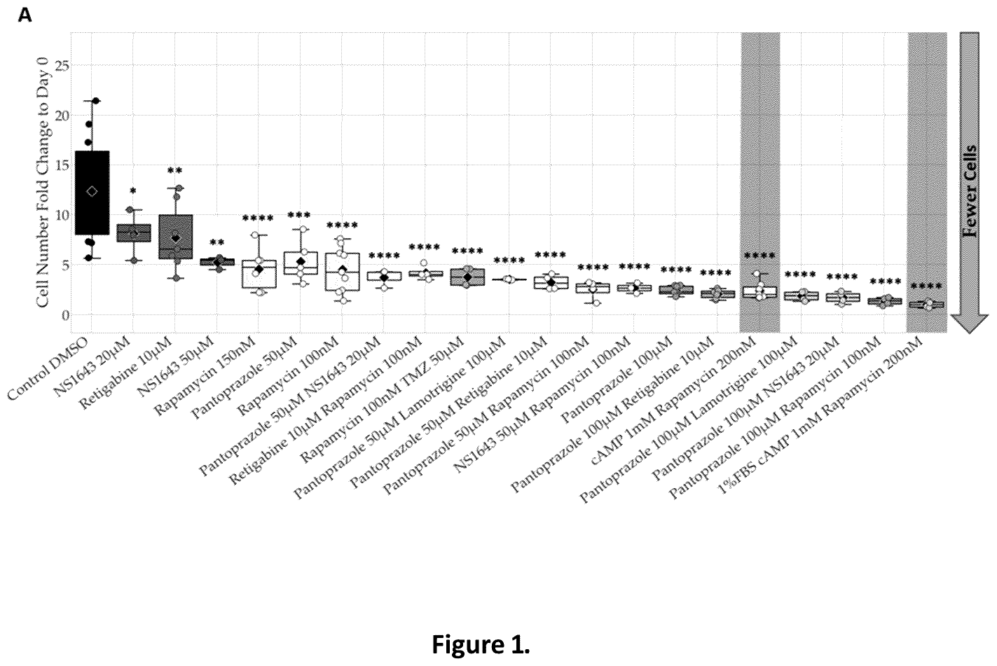

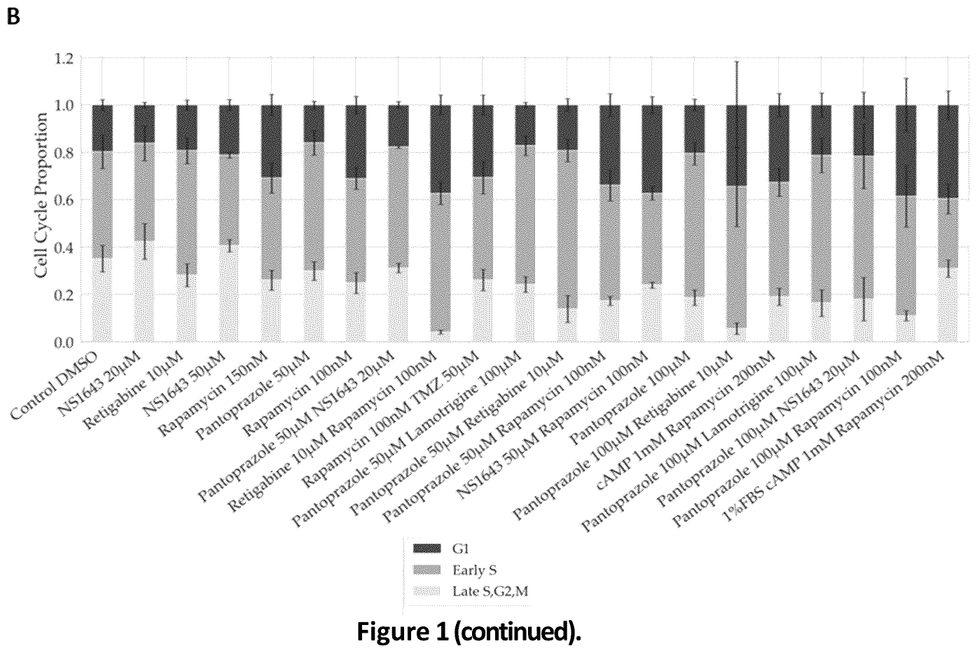

Many of the tested compounds showed a significant difference in proliferation from the controls after 6 days of treatment, but only a few of these were explored in more detail due to their novelty (Table 2). FIG. 1 shows the best of the individual and combination treatments chosen when compared to control at day 6. Note chlorzoxazone was not included in combination treatments in the NG108-15 analysis shown, due to its poor performance in the U87 cell line. The stacked bar graphs show the ratio of cells in different parts of the cell cycle as indicated by the FUCCI cell cycle reporter.

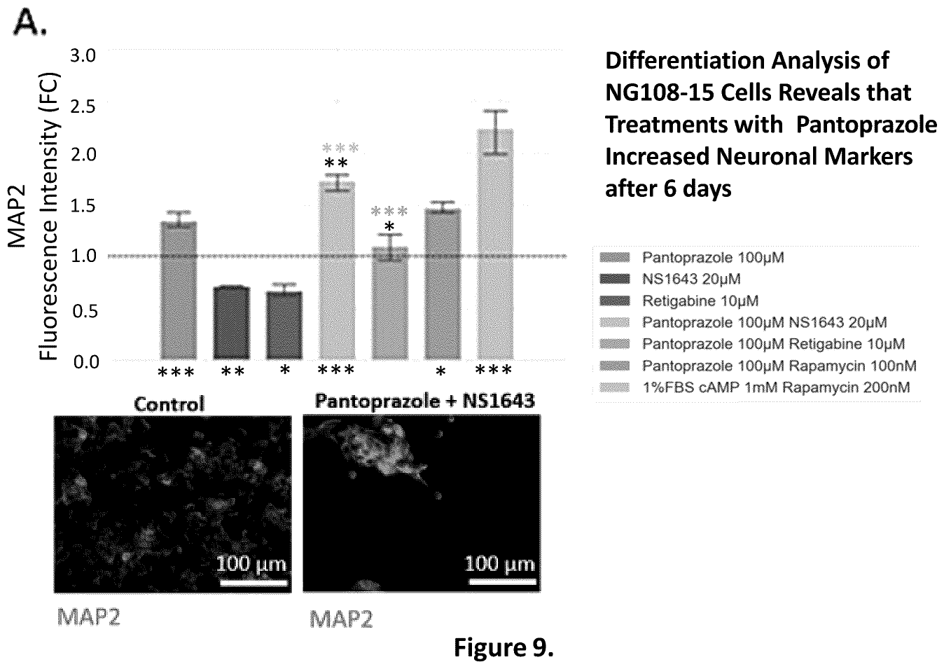

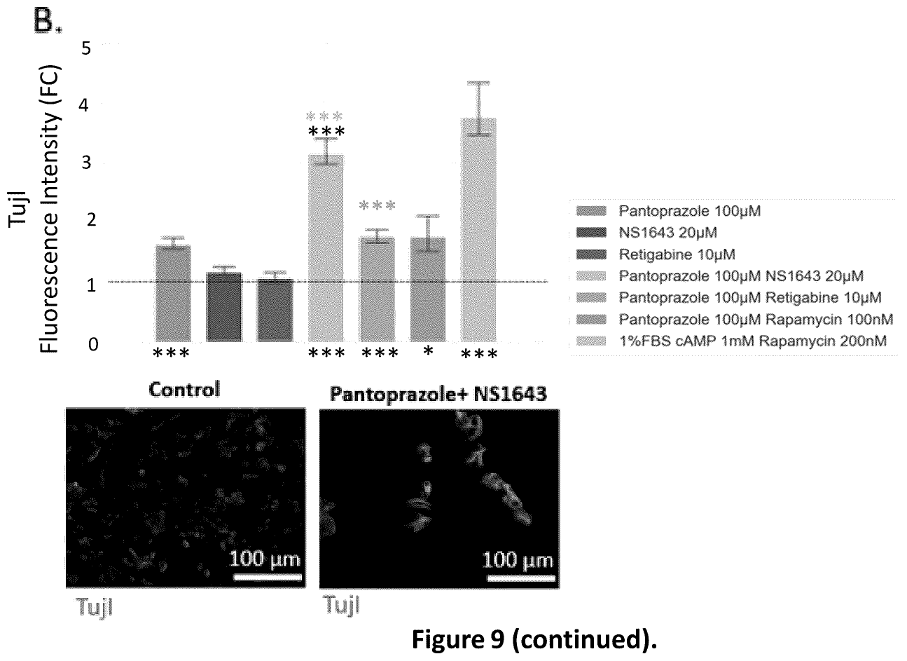

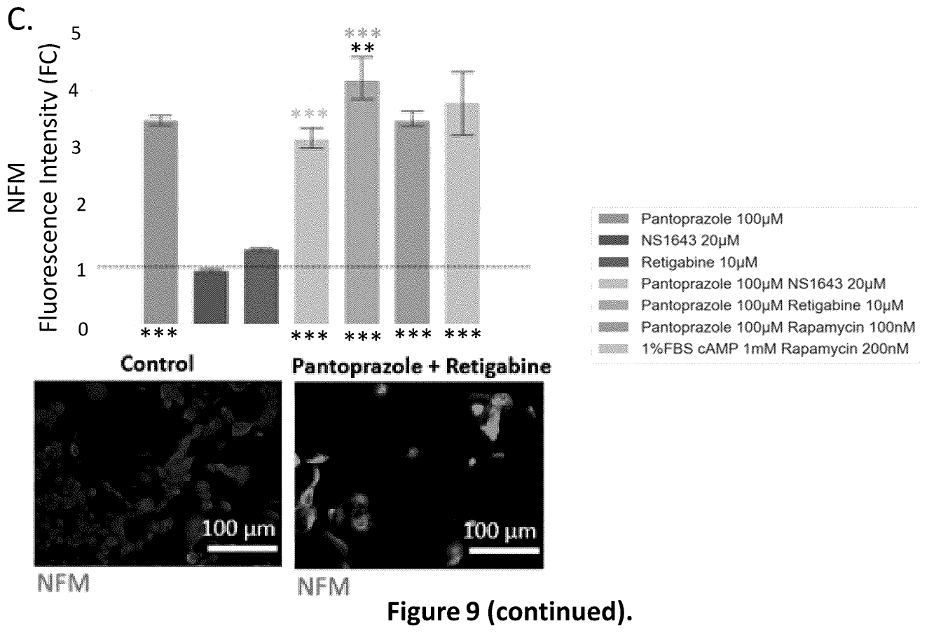

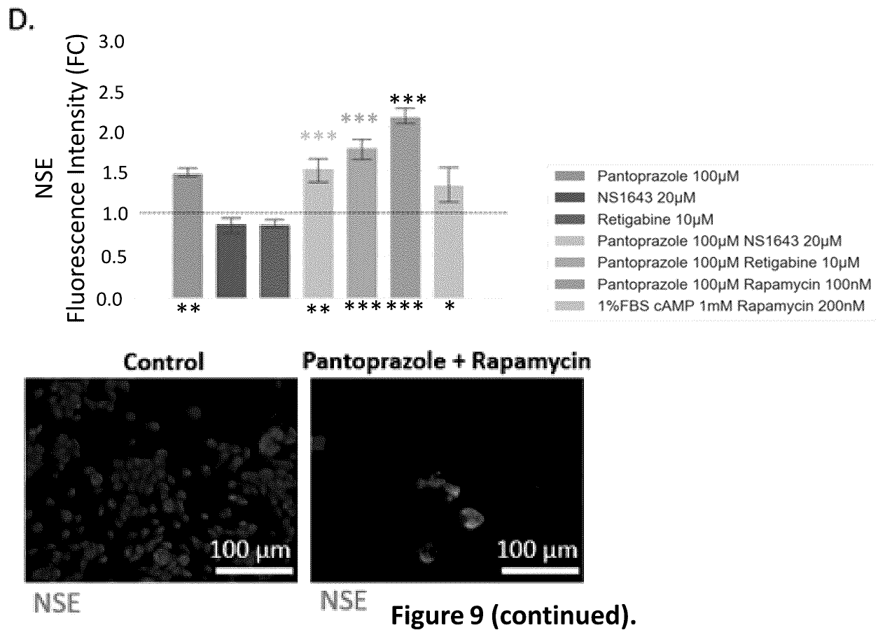

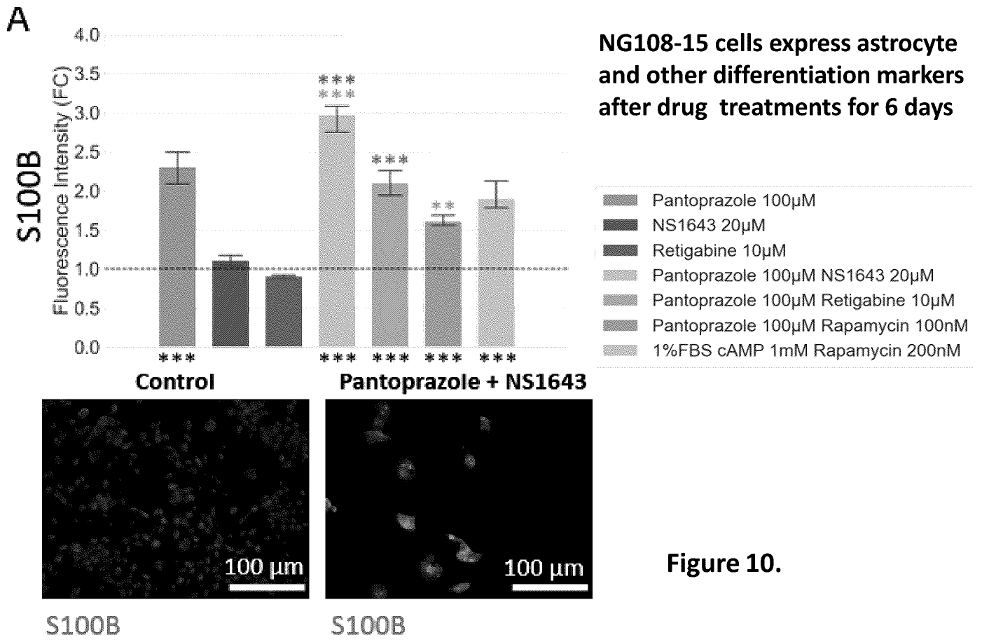

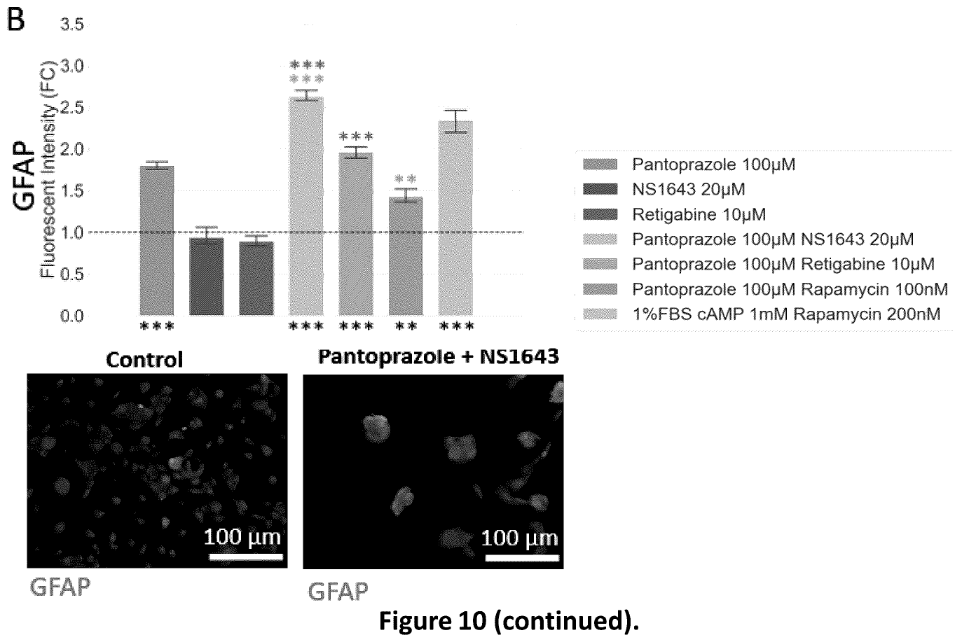

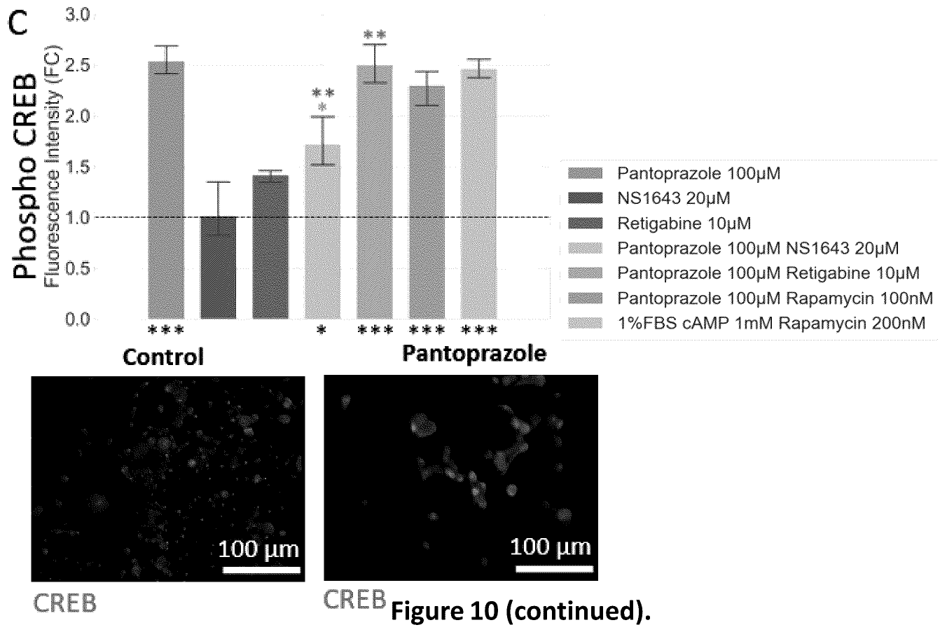

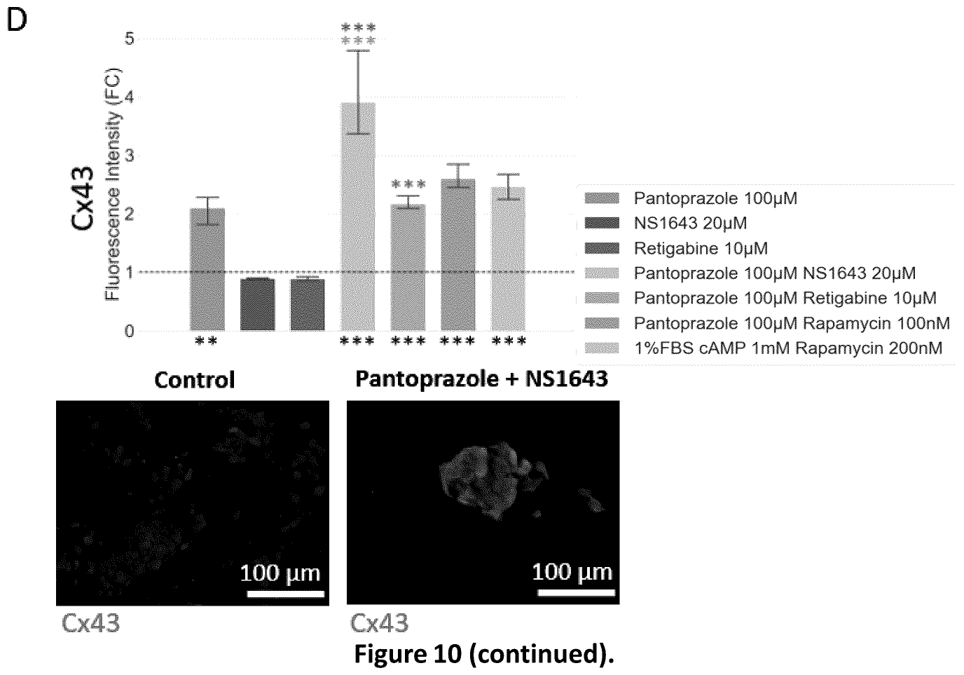

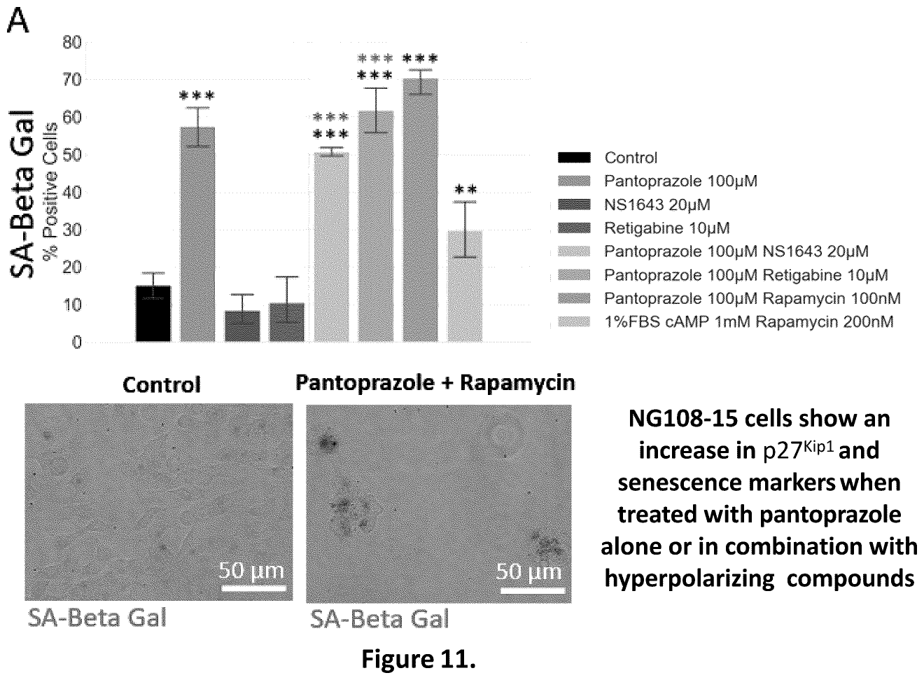

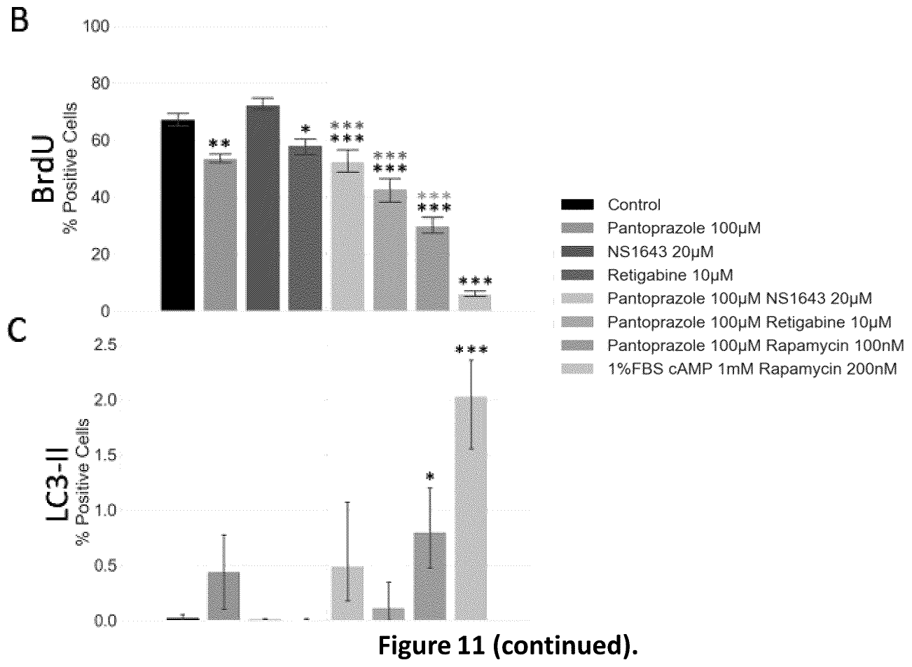

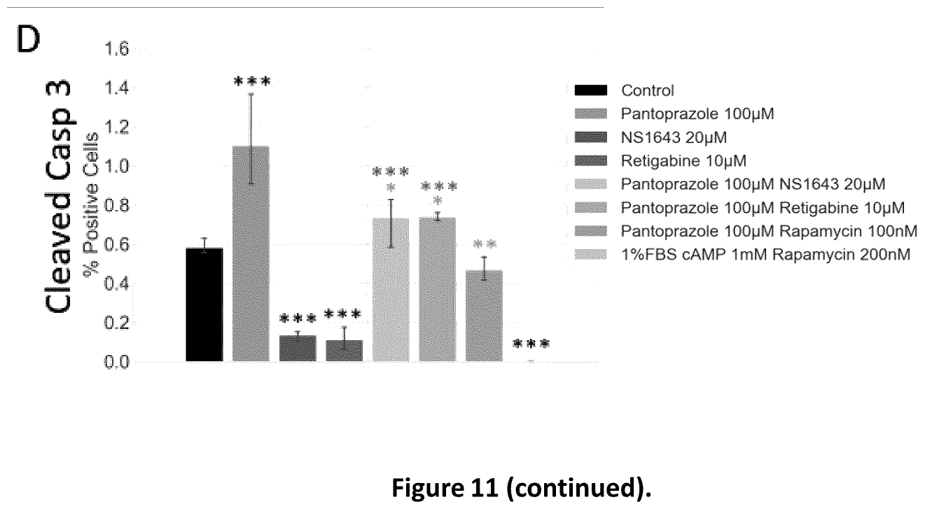

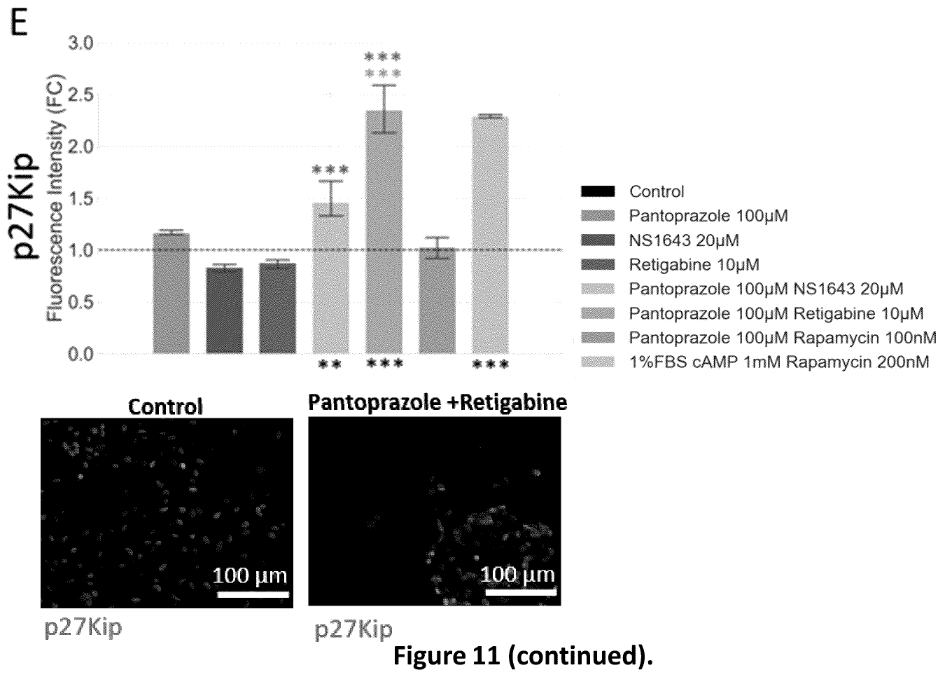

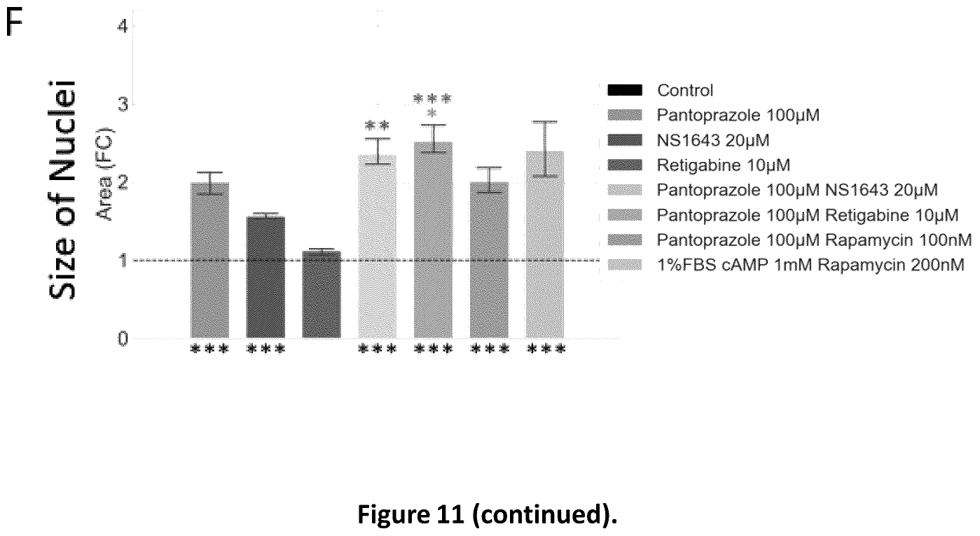

Proliferation of NG108-15 cells treated with NS1643 a human Ether-a-go-go (hERG) potassium channel activator at 20 μM and 50 μM alone were able to lower proliferation significantly about 1.6- and 2.4-fold decrease compared to control respectively (FIG. 1-A). In fact, NS1643 at 20 uM was very effective at lowing cell proliferation when combined with pantoprazole a proton pump inhibitor known to inhibit the expression of the vacuolar-ATPases (V-ATPases) in human gastric adenocarcinoma [79], this combination worked significantly better than pantoprazole or NS1643 alone, with a fold decrease of 7.2. NS1643 at 50 μM also significantly lowered proliferation when combined with rapamycin (autophagy inducer), with a 4.6-fold decrease and worked better than rapamycin or NS1643 alone. Retigabine, which opens the voltage-activated potassium channel Kv7 [80,81], significantly lowered cell proliferation by about 1.6-fold decrease compared to control, but its combination with rapamycin or pantoprazole worked better than any of those compounds alone with a 2.9- and 6-fold decrease as compared to control, respectively. Pantoprazole at 100 μM however, was the most effective compound alone (5.1-fold decrease) or in combination with lamotrigine which blocks voltage gated sodium channels [82], or NS1643, or rapamycin, with fold decreases compared to control of 6.8, 7.2, and 9.3 respectively. These three combinations worked better than one of the positive controls which consisted of a treatment of 1 mM cAMP with rapamycin at 200 nM in full serum media (5.1-fold decrease). This same positive control treatment is known to terminally differentiate these cells when in low serum [83], which was confirmed (FIGS. 9-10). However, the use of cAMP is problematic clinically due to its many off-target effects [84]. The cell cycle data in FIG. 11-B reveal that pantoprazole increases the proportion of cells in early S and that its combinations can also increase the proportion of cells in G1. Rapamycin treatment alone increased the proportion of cells in G1, while NS1643 treatment did not seem to affect the cell cycle proportion (FIG. 1B). It is worthwhile to note that these compounds were effective on NG108-15 cells, while temozolomide (TMZ), the standard glioblastoma treatment, was not, a situation found in many GBM cases [85].

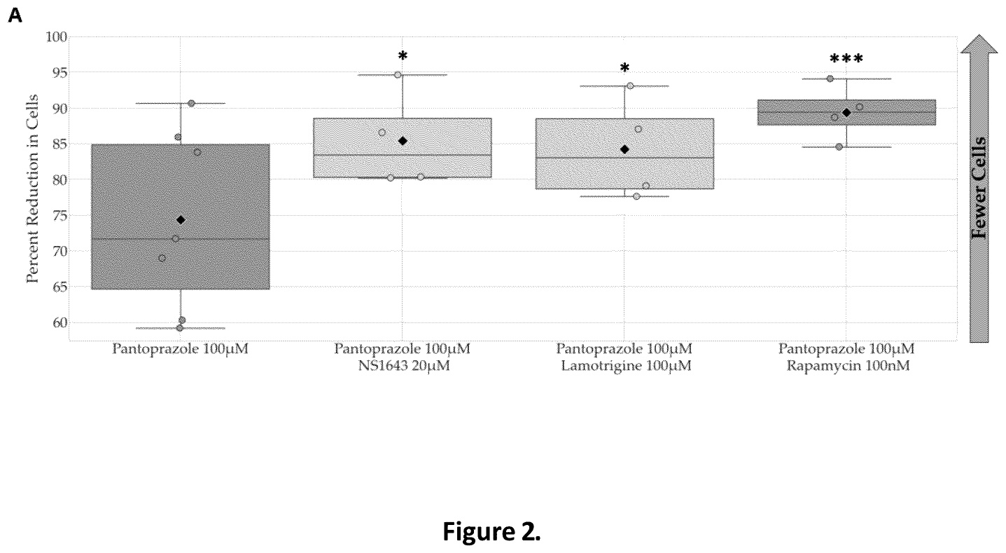

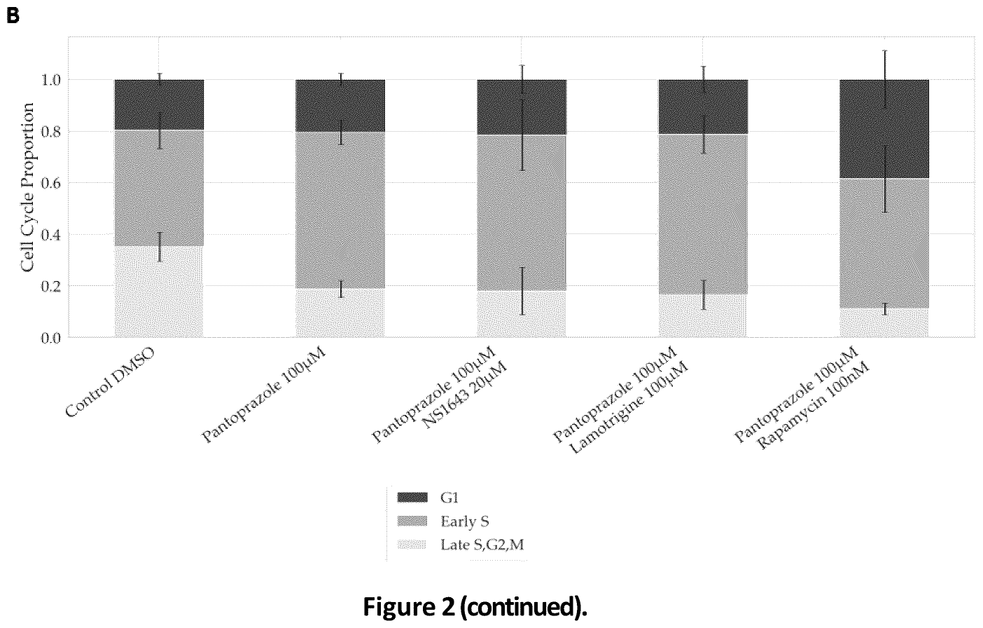

The combination of pantoprazole with lamotrigine at 100 μM, NS1643 at 20 μM, and rapamycin at 100 nM were the only combinations that showed significantly more efficacy than pantoprazole alone at reducing cell proliferation after 6 days of treatment, with the combination with rapamycin showing the most significant difference (FIG. 2-A). The reductions in cell number for these combinations compared to control were 85%, 86%, and 90% respectively. The proportion of cells in G1 and early S only slightly increased for pantoprazole treatments in combination with lamotrigine and NS1643, but the combination with rapamycin did increase the G1 proportion (FIG. 2-B). Taken together, these data reveal that several FDA approved drugs for human use, can be repurposed in combination and can significantly reduce cancer cell proliferation.

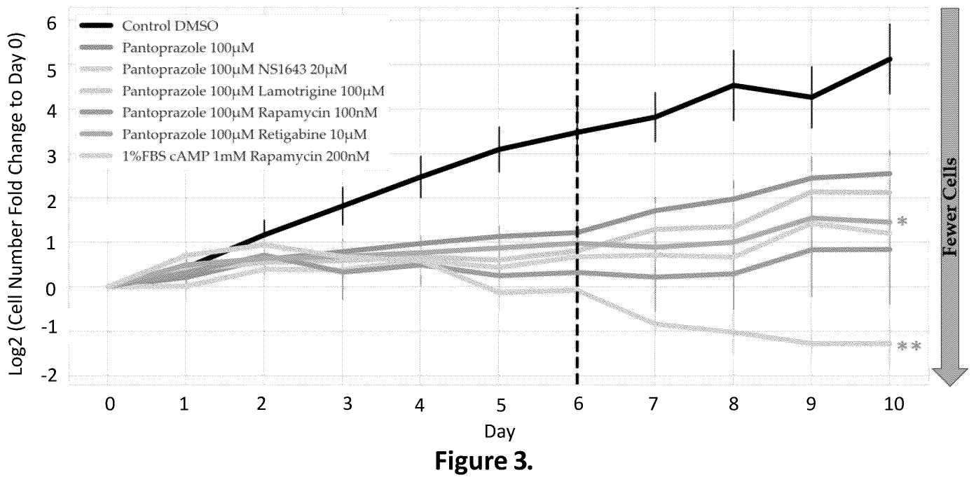

Hyperpolarizing drugs in combination with pantoprazole reduce NG108-15 proliferation after treatment is removed. To understand whether these treatments have a persistent effect on the cells (stop the proliferation of the cells even after the drugs were withdrawn), a recovery test was performed (FIG. 3). Cells were treated with the drugs for 6 days and then the drugs were removed (demarcated by the dashed line), after which the cells were cultured in media containing no drugs for another 4 days.

Although the three combinations with pantoprazole showed significantly less cell proliferation after 6 days of treatment, the cells showed some recovery after treatment was removed (FIG. 3). The slope of each combinatorial treatment from day 6 to day 10 was compared to pantoprazole alone. Pantoprazole in combination with retigabine was the only treatment combination that was significantly different, besides the positive control, which showed no recovery, indicative of cells that had terminally differentiated. One thing to note, was that the treatment of pantoprazole with retigabine, although not significantly different than pantoprazole at day 6, did show less cells than the treatment of pantoprazole with lamotrigine at day 10. Therefore, we decided to perform further analysis on this combination.

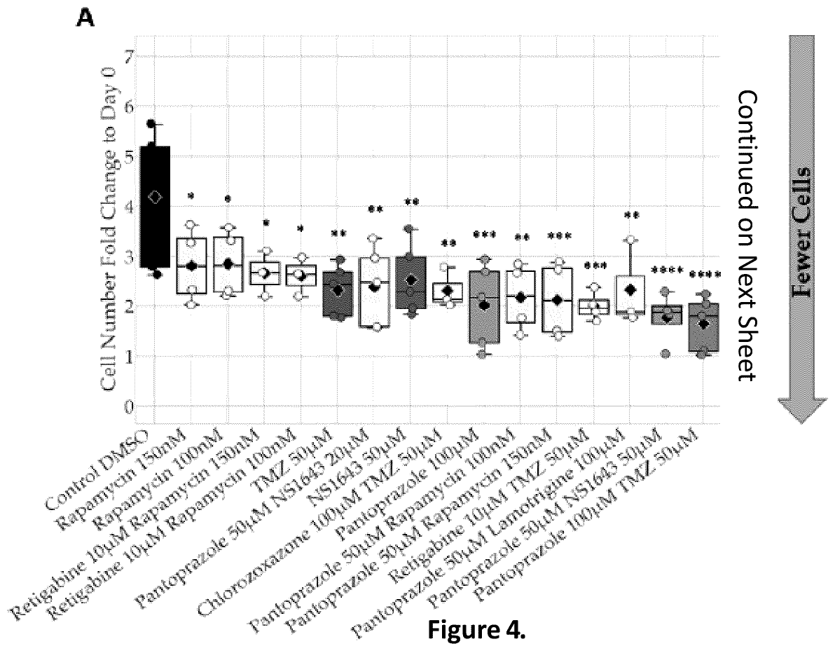

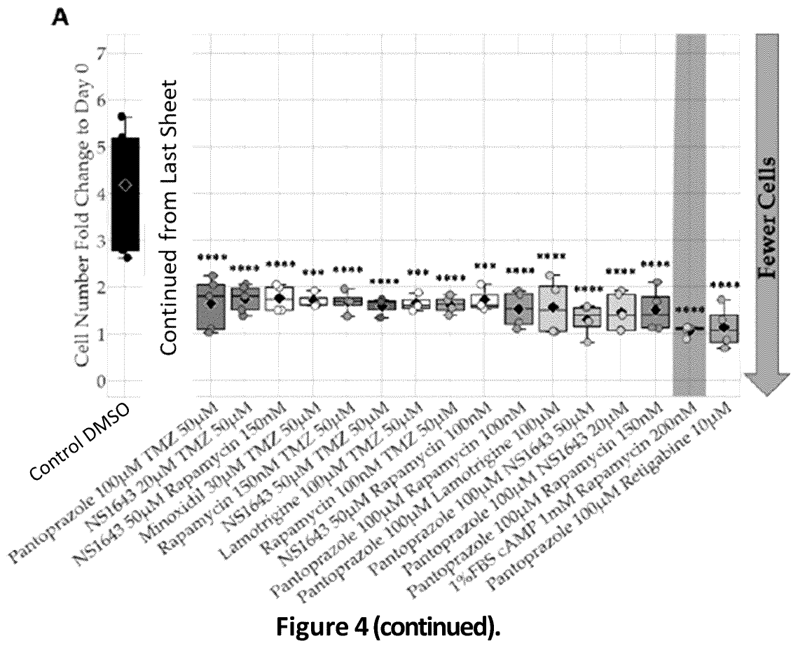

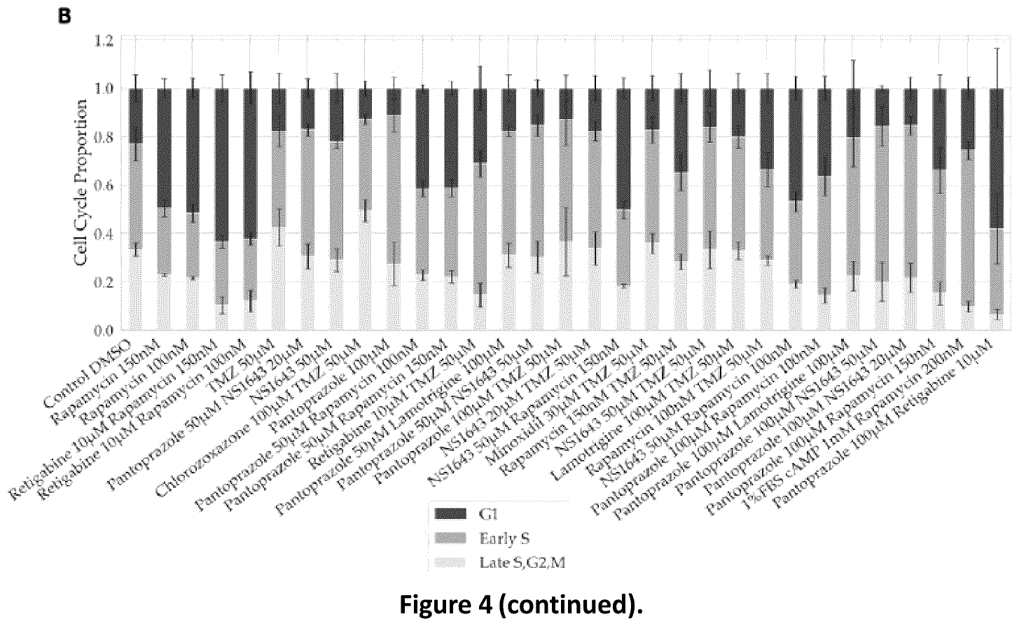

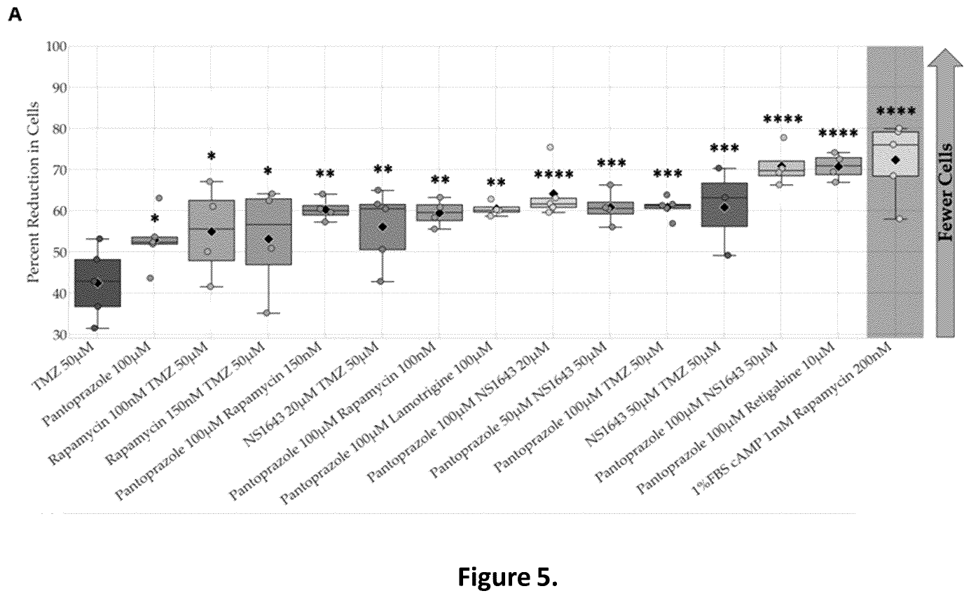

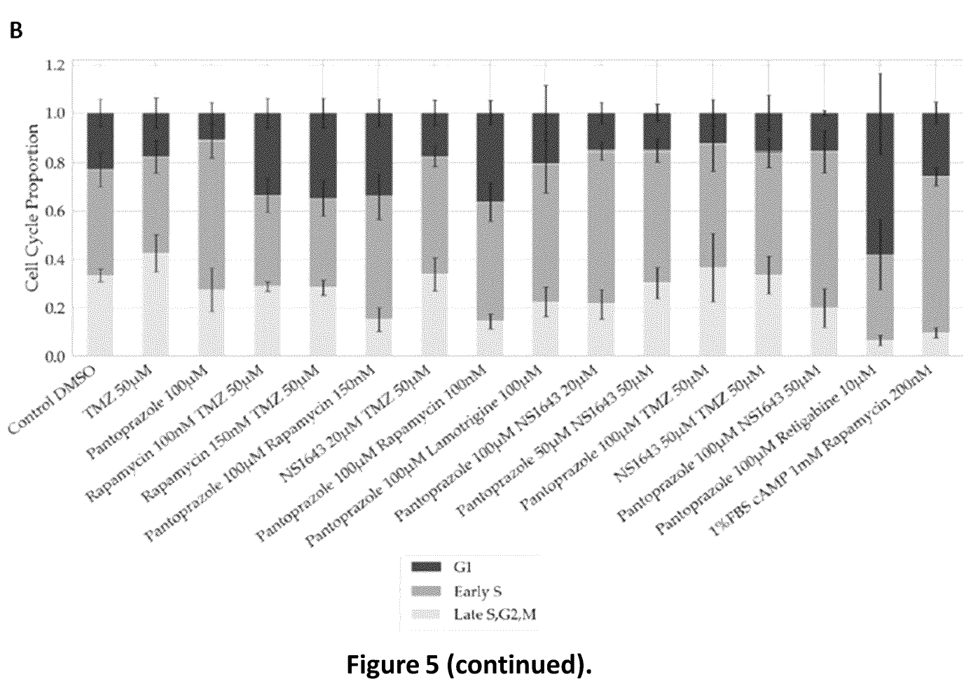

Many of the significant treatments in NG108-15 cells were also significantly effective in human glioblastoma U87 cells compared to the control after 6 days (FIG. 4). Notably, NS1643 at 50 μM significantly decreased cell proliferation as compared to control by 1.7-fold, but was much more effective when combined with pantoprazole, rapamycin, or temozolomide (TMZ) with a fold decrease to control of 3.3, 2.7, and 2.5 respectively. Pantoprazole also worked very well in this cell line with a significant percent reduction in cells to control of 54% and showed very significant differences in cell proliferation as compared to control when combined with rapamycin, retigabine, NS1643, lamotrigine, and TMZ with a percent reduction in cells of 60%, 72%, 72%, 61%, and 61% respectively (FIG. 5-A). TMZ was very effective at reducing cell number as compared to control in U87 cells (43% decrease), but combinations with rapamycin, pantoprazole, or NS1643 significantly increased the effectiveness up to 55%, 61%, 61% reduction in cells compared to control respectively (FIG. 5-A). The cell cycle data in FIG. 4-B shows that some of the most effective combinations increased the G1 and early S proportion of cells but not all. Pantoprazole showed its characteristic increase in the early S proportion of cells and rapamycin an increase in the G1 proportion seen in the NG108-15 cells. TMZ and NS1643 treatment did not show a large change in proportion of cells in each stage of the cell cycle as compared to control. TMZ in combination with rapamycin increased the proportion of cells in G1 and combination with NS1643 increased the proportion of cells in early S as compared to TMZ alone (FIG. 5-B). Pantoprazole combinations consistently showed a larger proportion of cells in early S as compared to TMZ alone with a complimentary decrease of cells in late S, G2, and M (FIG. 5-B).

Unfortunately, we were notable to obtain an accurate proportion of cells in each cell cycle stage for the best treatment in U87 cells, pantoprazole with retigabine, due to an autofluorescent aggregation in the cytoplasm that obscured FUCCI reporter signal.

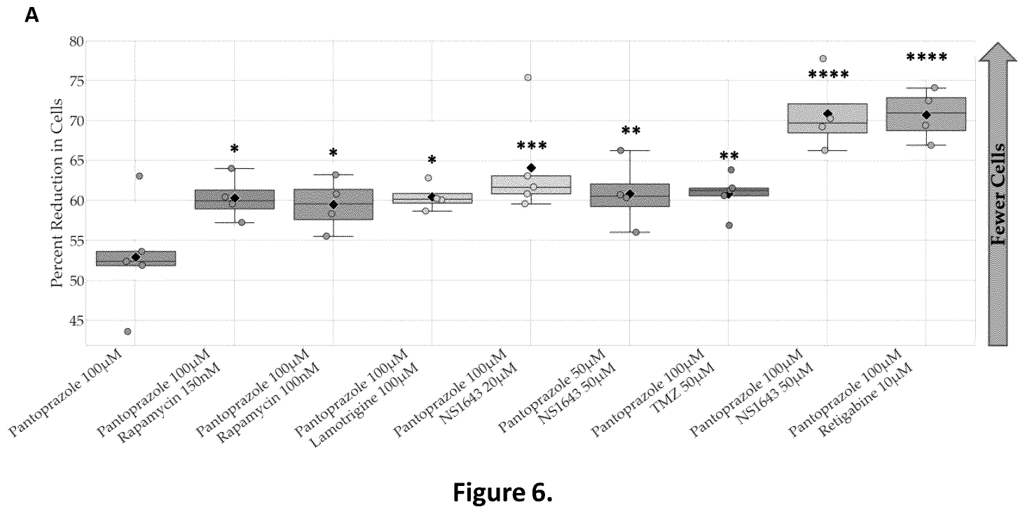

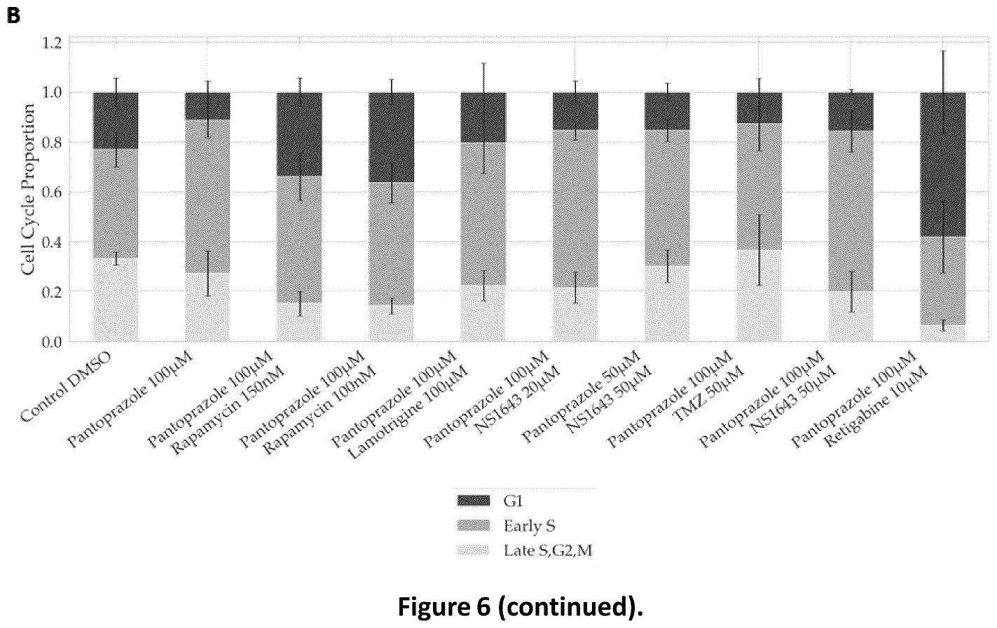

The same combinations that were significantly better than pantoprazole in NG108-15 cells were also significant in U87 cells along with the added combinations of TMZ or retigabine (FIG. 6-A). Pantoprazole in combination with rapamycin, lamotrigine, NS1643, TMZ or retigabine were all significantly better than pantoprazole alone. The most significant combinations were with NS1643 or retigabine, 1.6-fold decrease than pantoprazole alone. The combination of pantoprazole with NS1643 was so effective that cutting the pantoprazole concentration by half and combining it with NS1643 at 50 μM was significantly more effective than pantoprazole at 100 μM alone (1.2-fold decrease). The characteristic increase in G1 when pantoprazole was combined with rapamycin and the combination with NS1643 increased the proportion of the cells in early S, with a complimentary decrease in late S, G2, and M (FIG. 6-B). Thus, NS1643, retigabine, rapamycin, lamotrigine, and pantoprazole are also effective in a human glioblastoma cell line, and NS1643 or pantoprazole potentiate the action of the standard TMZ treatment.

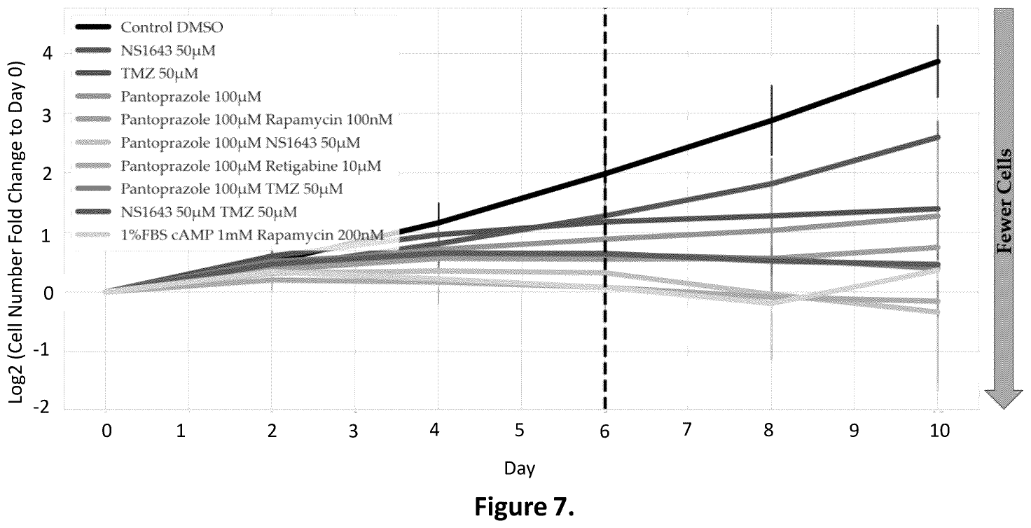

Bioelectric drug combinations including pantoprazole reduce U87 proliferation after treatment is removed. The combination of pantoprazole with rapamycin shows a similar recovery slope after day 6 as pantoprazole alone (FIG. 7). However, combinations of pantoprazole with retigabine or NS1643 at 50 μM show reduced recoveries compared to pantoprazole alone, although not significantly so. TMZ treatment alone only showed a slight increase in proliferation after treatment was removed, and NS1643 alone showed a high increase in proliferation. However, the combination of NS1643 with TMZ did not show an increase in proliferation after removal of treatment, but the recovery slope was not significantly different than TMZ alone. Surprisingly the positive control using cAMP in combination with rapamycin started proliferating after day 8, revealing that the bioelectric drugs have a more stable effect on the cells than even the powerful cAMP signal.

Electrophysiology of NG108-15 Cells Show Changes in Resting Membrane Potential Induced by Treatment. Electrophysiological measurements were used to determine the change in resting membrane potential caused by treating NG108-15 cells with the compounds that significantly reduced cell proliferation as compared to control immediately after application. Untreated cells were patched, baseline measurements were taken and then the drugs were added and the changes in the cell Vₘₑₘ were recorded.

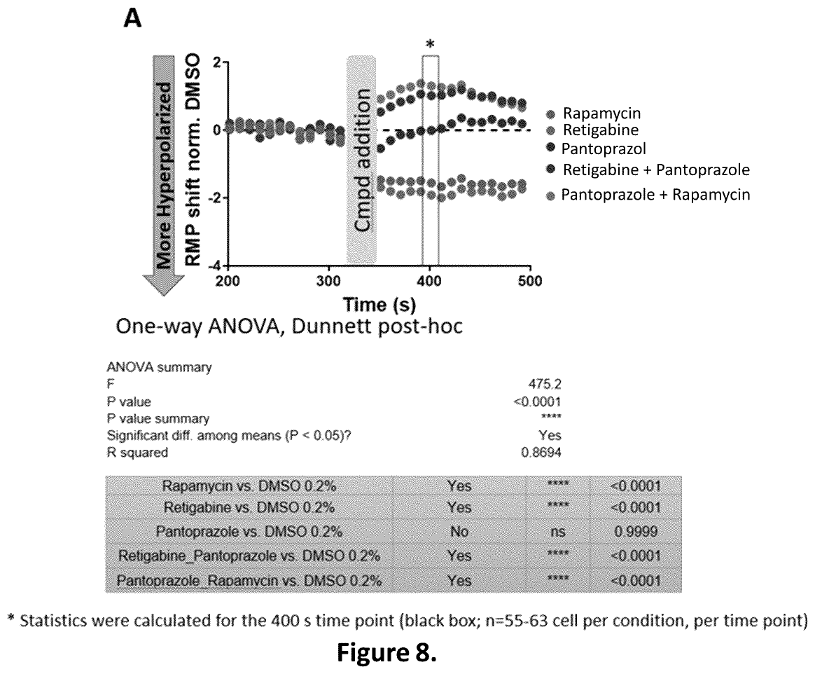

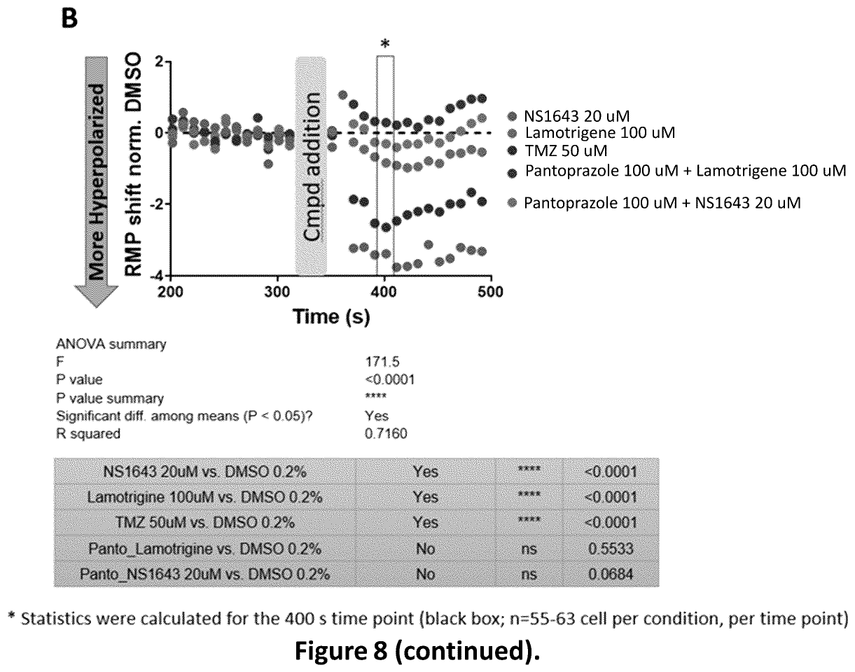

Rapamycin, retigabine, NS1643, TMZ, and lamotrigine all significantly hyperpolarized the cells as compared to the control (FIG. 8). Retigabine, lamotrigine, and NS1643 are known hyperpolarizing agents, but TMZ has been published to depolarize glioma cells [86]—an effect opposite to what was observed in NG108-15 cells. Rapamycin also hyperpolarized the NG108-15 cells, a novel effect suggesting that existing cancer drugs could have bioelectric mechanisms of action that are not yet recognized. Surprisingly, while pantoprazole did not have an immediate effect on the membrane potential of the cells, its combination with retigabine and rapamycin, which both individually hyperpolarize the cells, instead depolarized the cells. The combination of pantoprazole with lamotrigine and NS1643 did not observably change the membrane potential as compared to control.

NG108-15 cells express neuronal markers with drug treatments after 6 days. The next question was whether, in addition to the effects on proliferation, such treatments also exerted a differentiating influence, which could be beneficial with respect to future behavior of treated cells in vivo. Differentiation markers for neuronal lineage were used to stain NG108-15 cells incubated for six days with the most effective treatments observed in the proliferation data (FIG. 9). Treatment with pantoprazole at 100 μM combined with NS1643 at 50 μM or with rapamycin at 100 nM consistently showed a significant increase in neuronal differentiation markers, including Microtubule Associated Protein 2 (MAP2) [87], Neuron-Specific Class III beta-Tubulin (TujI) [88], Neuron-Specific Enolase (NSE) [89], and Neural Filament Medium Chain (NFM) [90]. The combination of pantoprazole with retigabine at 10 μM showed increases in all but MAP2. Combinations of pantoprazole with either NS1643 or retigabine showed a significant increase in neuronal markers compared to either drug alone. The combination of NS1643 with pantoprazole showed higher levels of both MAP2 and TujI, than for pantoprazole alone. Retigabine in combination with pantoprazole showed higher immunoreactivity of NFM as compared to pantoprazole alone. These observations suggest that these treatments are pushing the treated cells toward a more differentiated state.

Staining cells treated for 6 days with of S100 calcium binding protein B (S100B) [91] and Glial Fibrillary Acidic Protein (GFAP) [92] markers revealed differentiation of NG108-15 cells towards an astrocytic/oligodendrocytic or astrocytic lineage respectively (FIGS. 10-A and -B). Both markers were significantly upregulated in all combination treatment groups as well as in pantoprazole alone. The combination of NS1643 with pantoprazole showed significantly higher S100B and GFAP immunoreactivity than pantoprazole. All treatments but NS1643 alone showed a significant increase in CREB, known to play an important role in driving differentiation [15,93-96](FIG. 10-C). Pantoprazole alone along with its combinations also showed an increase in connexin 43 (Cx43) expression, a known marker for glioblastoma differentiation [97].

The next question was whether any treatments induce senescence, as this would be an important outcome with respect to the course of the malignancy in vivo. Treatments of NG108-15 cells with pantoprazole alone or in combination with NS1643, retigabine, and rapamycin all showed a very significant increase in a senescence-associated beta-galactosidase activity stain [98](FIG. 11-A). To explain this increase, the level of p27^{Kip1} was investigated because the level of p27^{Kip1} is known to inhibit the cell cycle and cause senescence [99-106](FIG. 11-E). Immunoreactivity levels of p27^{Kip1} in all combined treatments except for pantoprazole with rapamycin were elevated. To confirm the senescence phenotype, the size of the nuclei were investigated and found to be significantly larger in the pantoprazole, NS1643, and pantoprazole in combination with NS1643, retigabine, or rapamycin treated groups (FIG. 11-F). The cells were tested for cleaved caspase 3 (FIG. 11-D), a marker of apoptosis and LC3-II (FIG. 11-C), a marker of autophagy. The levels for both these markers were found to be very low after 6 days of treatment, but there was a significant increase in cleaved caspase 3 staining for cells treated with pantoprazole or pantoprazole with NS1643, retigabine, or rapamycin. Surprisingly, treatment with retigabine or NS1643 was observed to decrease the cleaved caspase 3 positive cells significantly as compared to control. In addition, the LC-II stain only showed a significant increase in cells that were treated with rapamycin, as was expected (FIG. 11-C). Proliferation in NG108-15 cells as measured by BrdU incorporation showed a significant decrease with treatments of pantoprazole alone, retigabine alone, and combinations of pantoprazole with NS1643, retigabine, and rapamycin after 6 days (FIG. 11-B), agreeing with the live cell counts obtained in FIG. 1-A. Thus, the combinatorial treatments that resulted in the lowest cellular proliferation also showed increased markers for senescence and did not show a large fraction of apoptotic or autophagic cells.

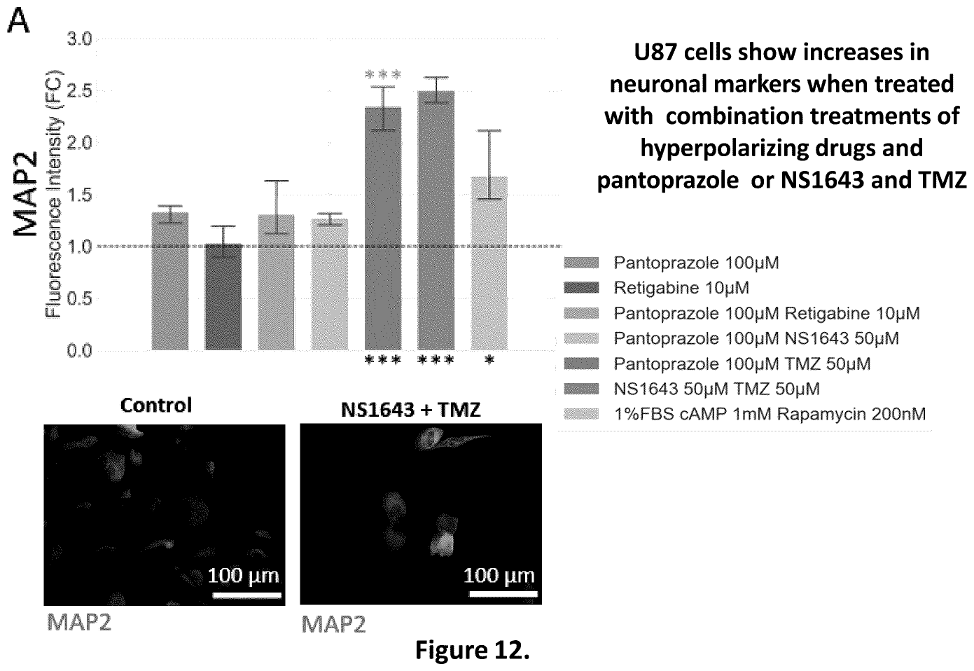

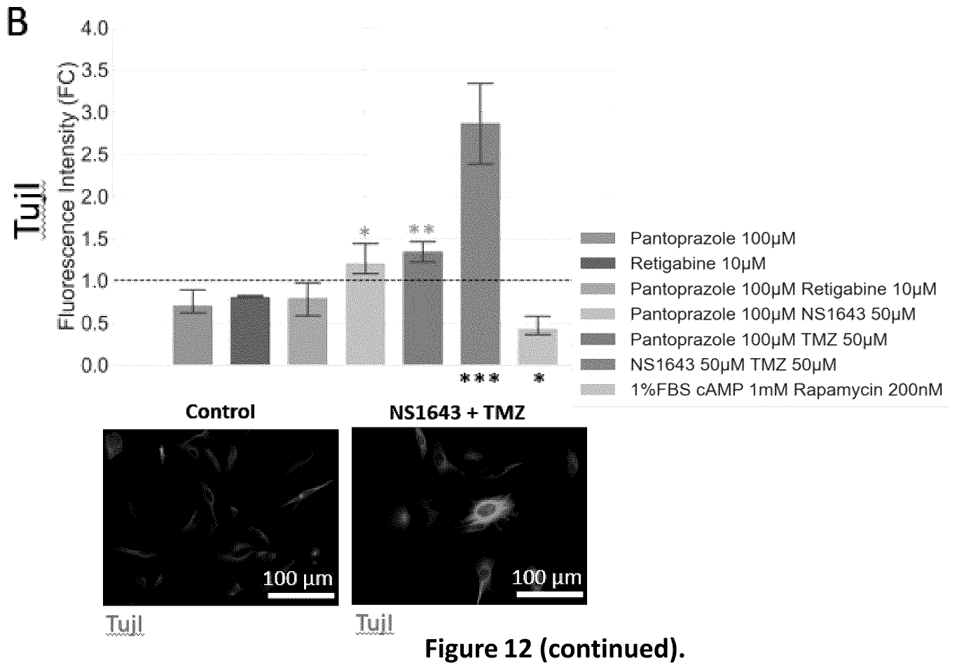

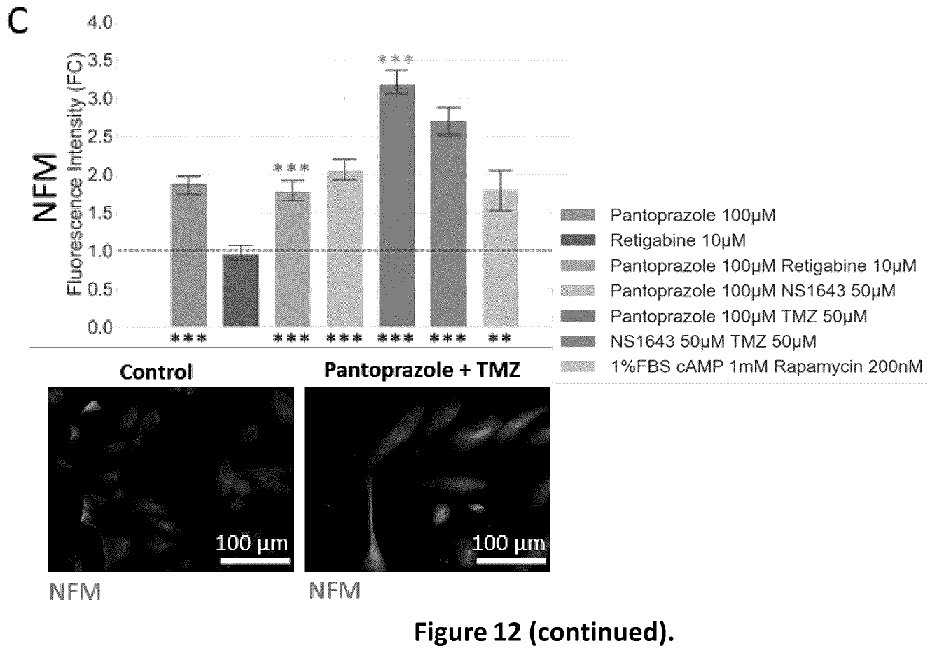

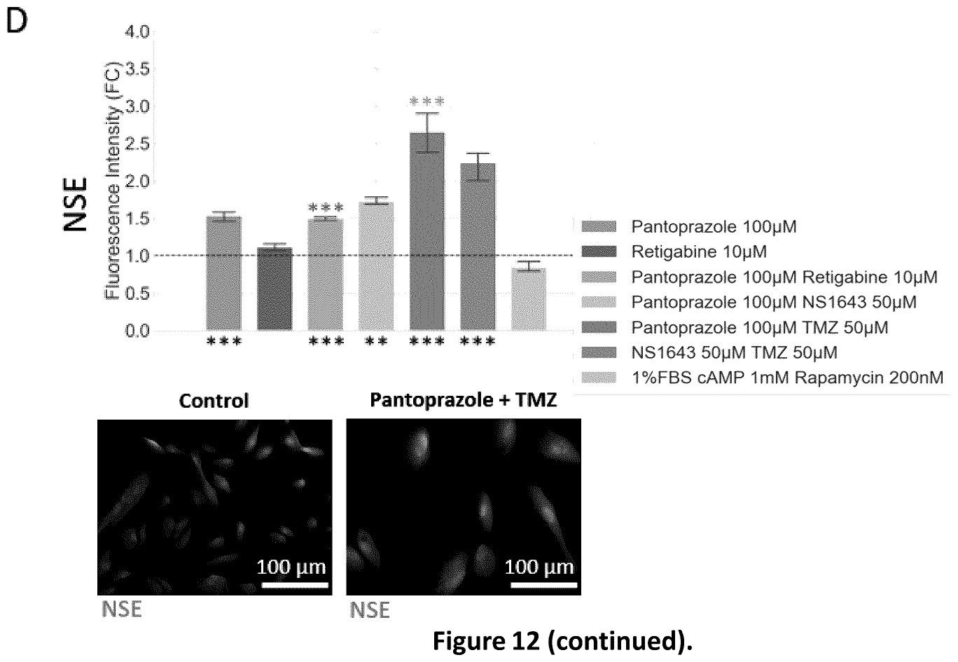

U87 cells showed a significant increase in neuronal markers for the combination treatments with TMZ as compared to control (FIG. 12). NFM and NSE, similar to what was seen in NG108-15 cells, were also increased in cells treated with pantoprazole or combination treatments with pantoprazole and NS1643 or retigabine (FIG. 12-C). TujI was only highly elevated in the U87 cells treated with NS1643 and TMZ (FIG. 12-B). MAP2 showed high levels of immunoreactivity when cells were treated with combinations of TMZ and NS1643 or pantoprazole (FIG. 12-A). It should be noted that cell staining was highly heterogeneous, with some cells staining more brightly than others with the same treatment.

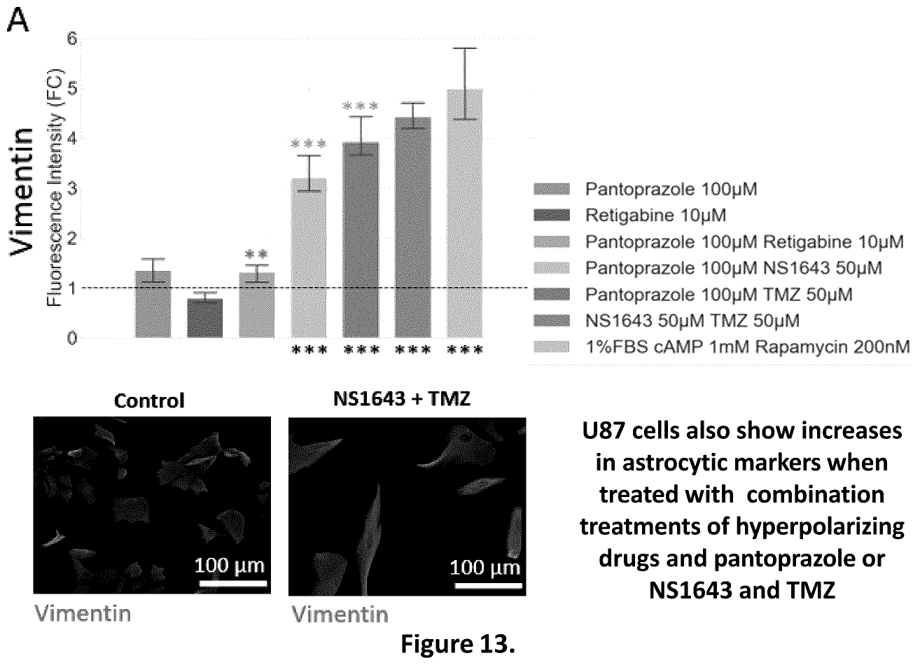

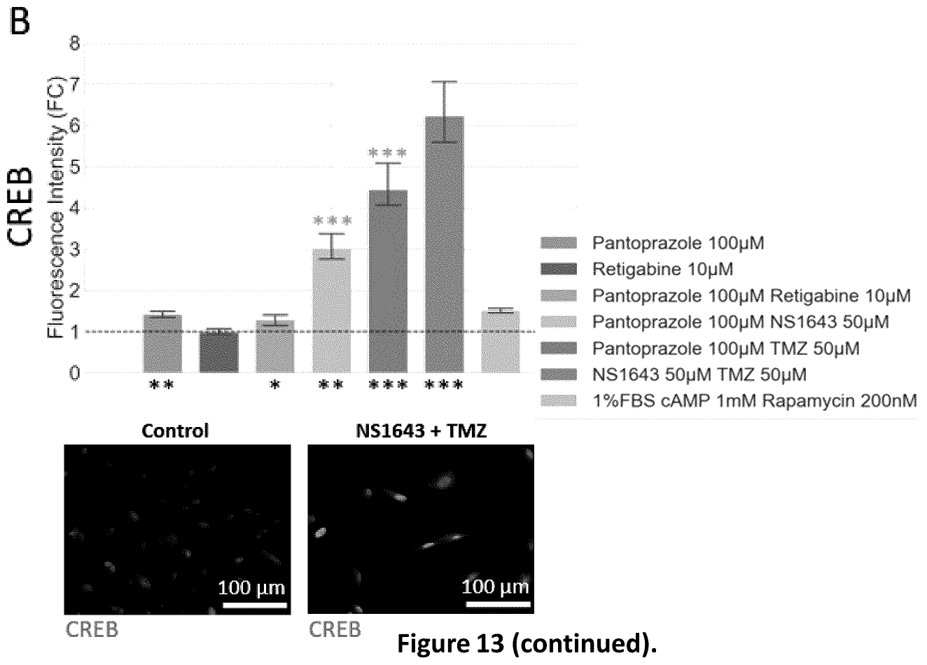

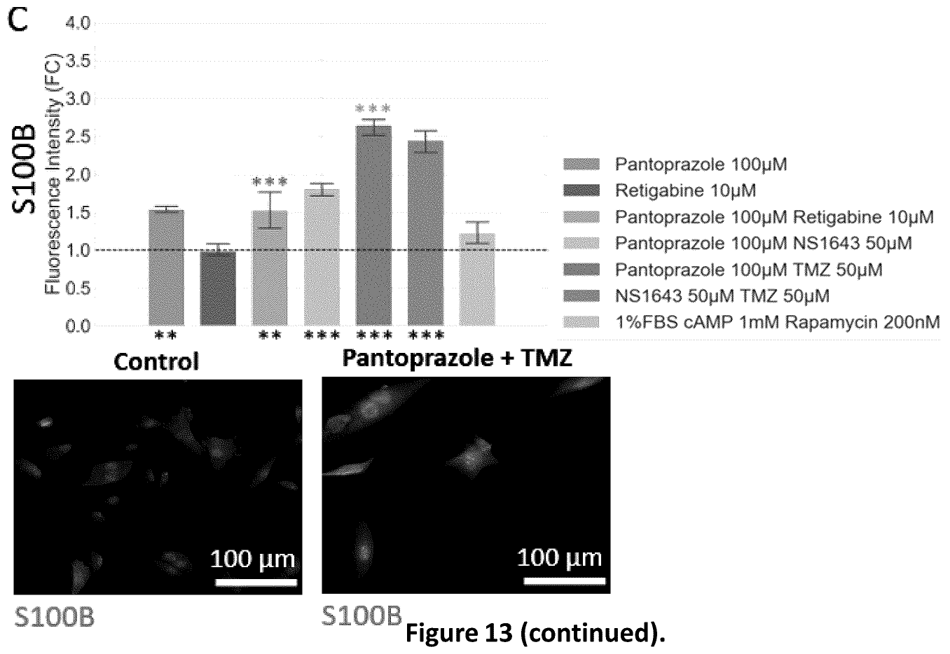

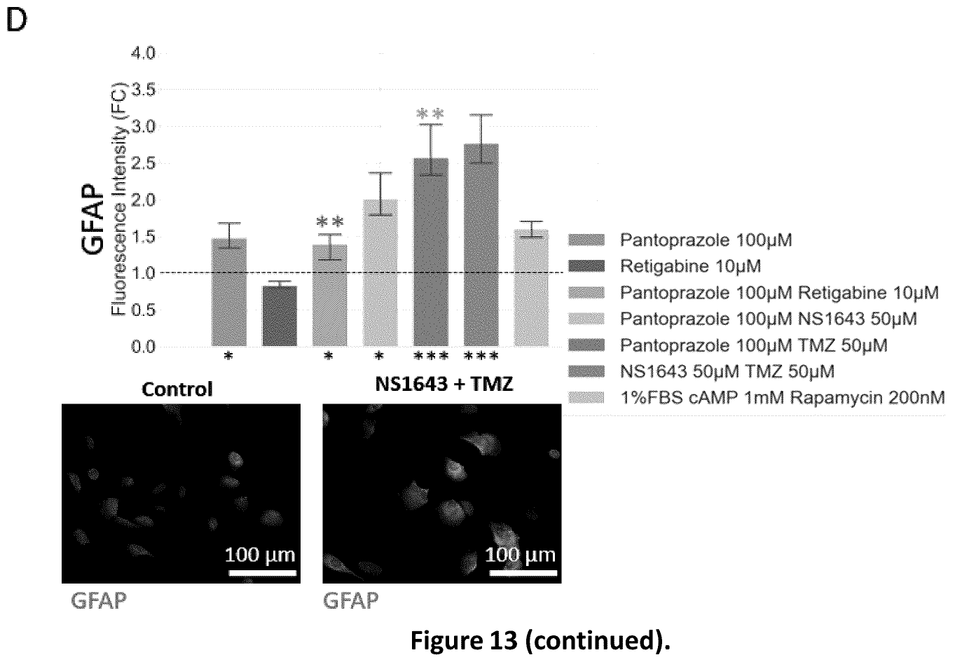

Astrocytic differentiation markers were increased in treatments with pantoprazole and pantoprazole in combination with NS1643, retigabine, rapamycin, and TMZ as well as NS1643 in combination with TMZ (FIG. 13). Vimentin, a known marker for astrocytes [107,108], was significantly increased when cells were treated with pantoprazole in combination with NS1643, TMZ, and rapamycin and with NS1643 in combination with TMZ (FIG. 13-A). CREB (FIG. 13-B), a known marker for differentiation and S100B (FIG. 13-C) and GFAP (FIG. 13-D), markers for astrocytic differentiation, also were increased by these same treatments (FIG. 13-B). These results agreed well with was found in NG108-16 cells.

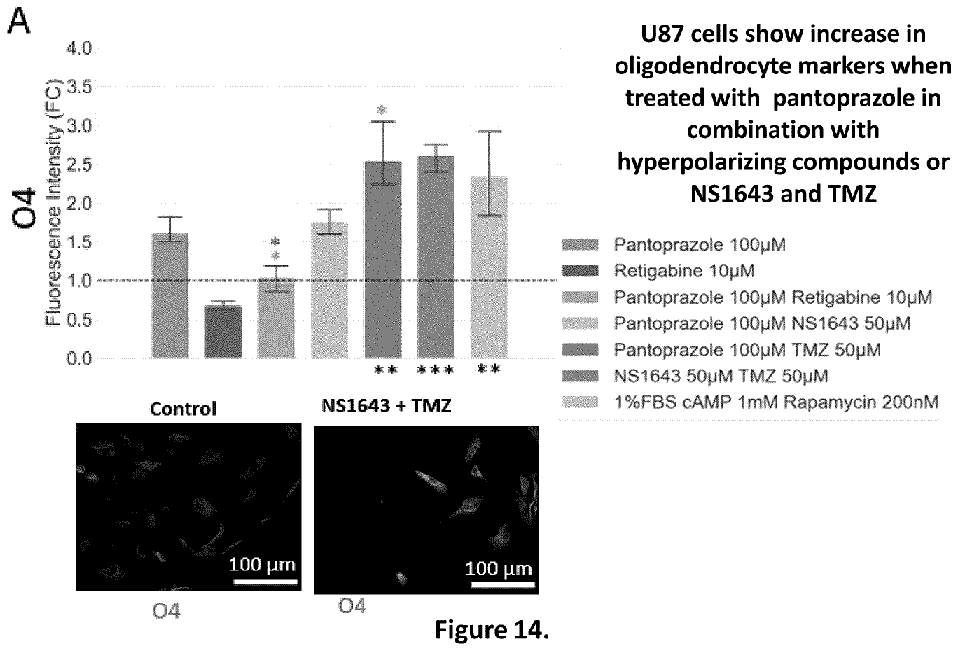

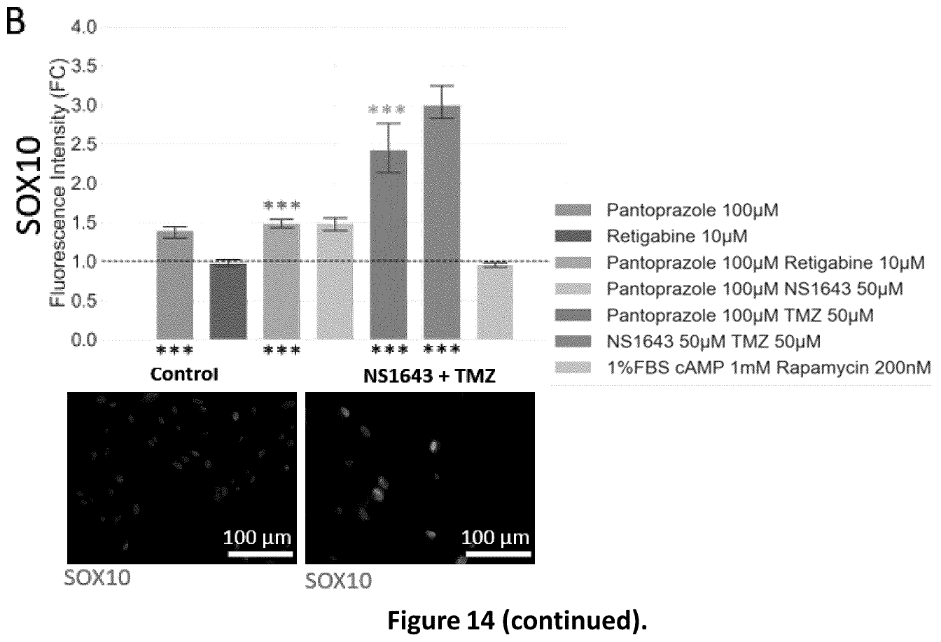

U87 cells showed increases in 04 (FIG. 14-A) for all treatments except those containing retigabine. Sox10 levels (FIG. 14-B) also increased for all treatments, excluding retigabine alone. Taken together, the above data suggest that the differentiating activity of these treatments are not specific to one species or one type of cell line.

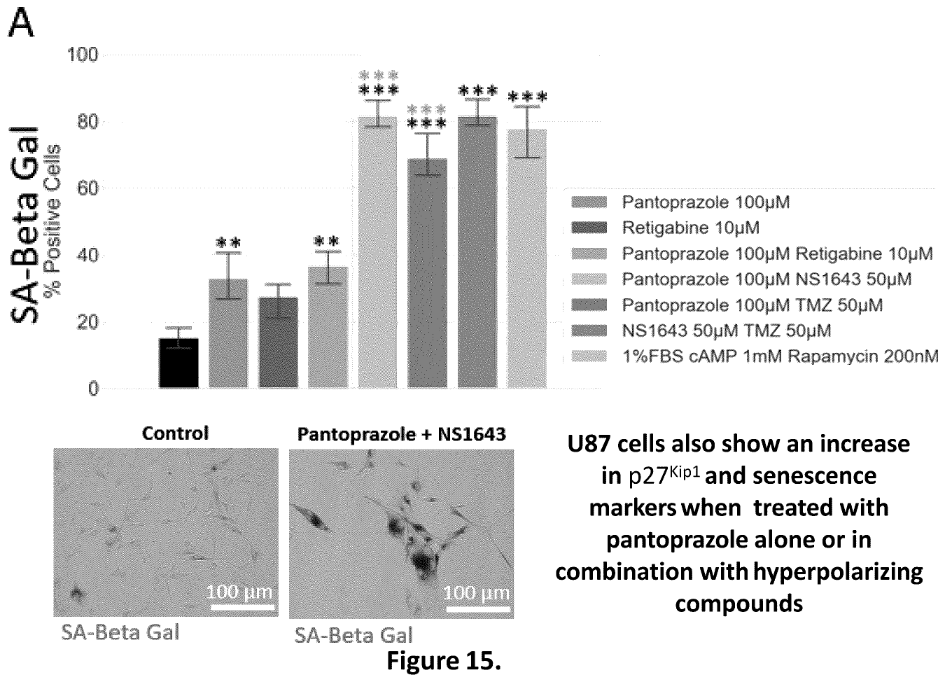

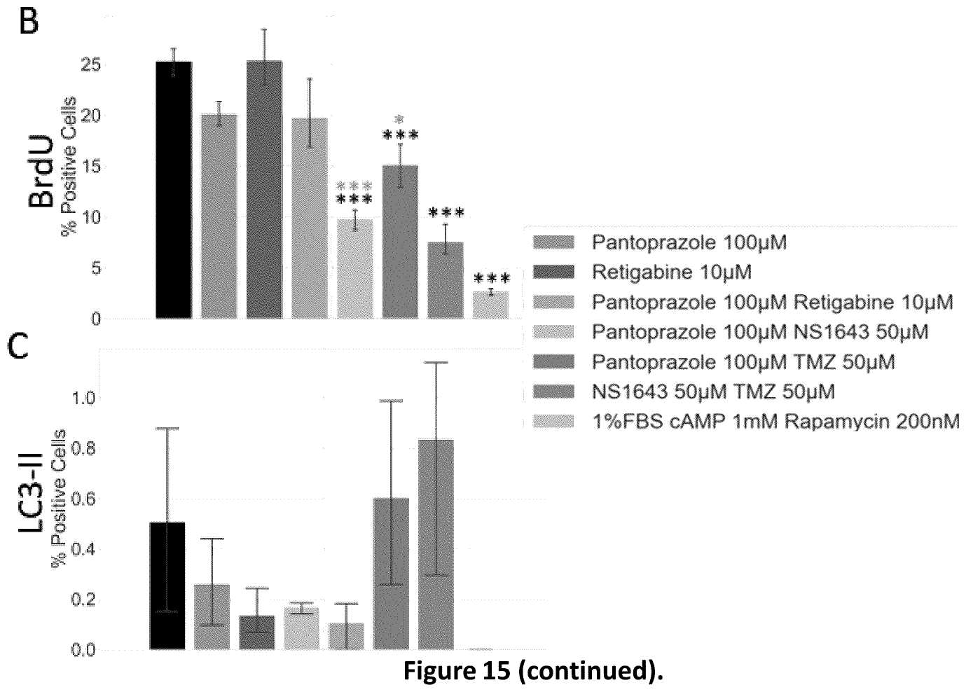

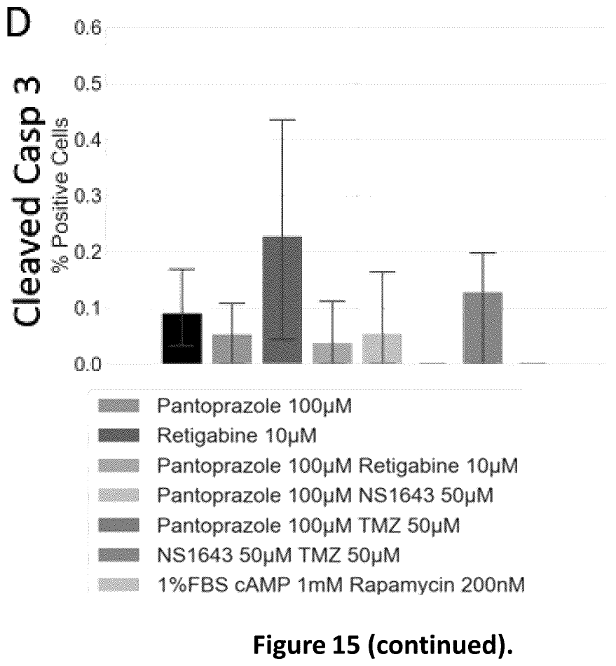

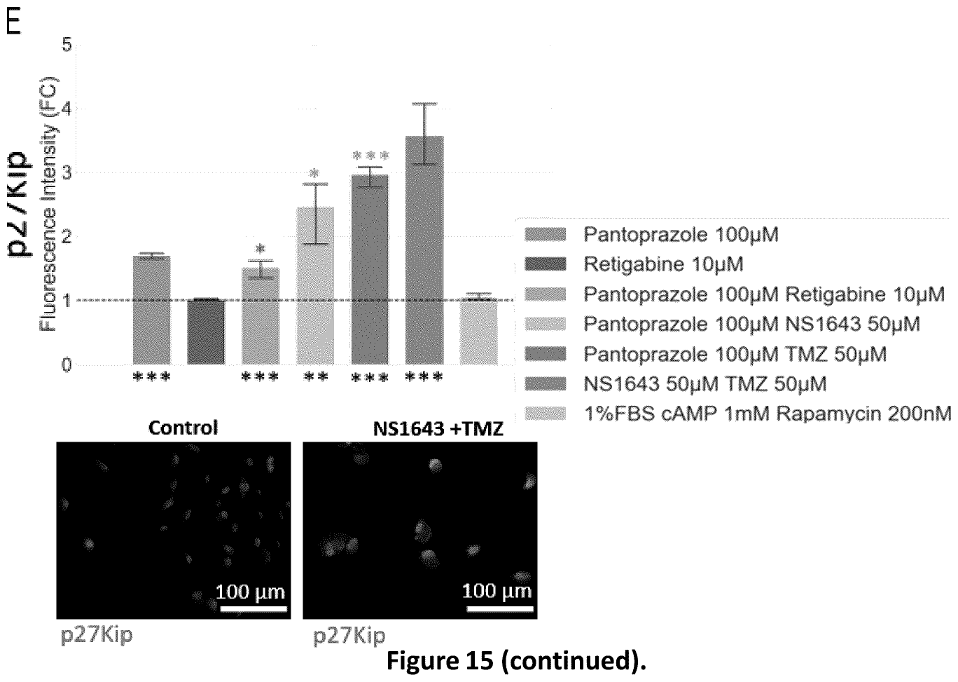

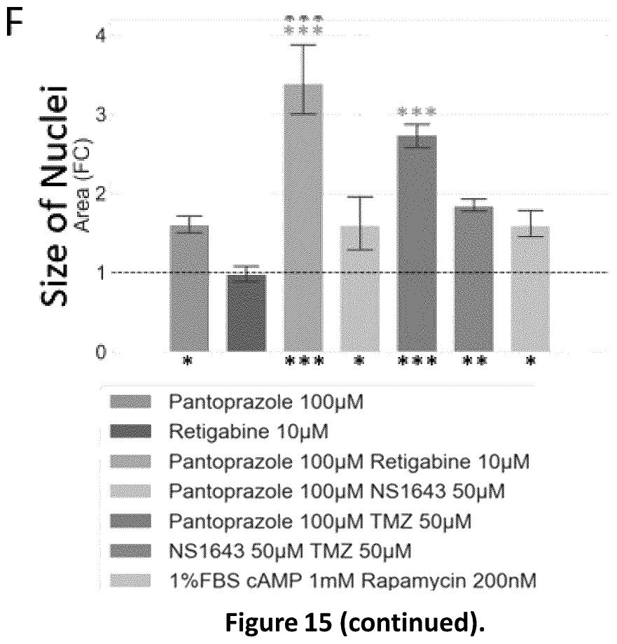

U87 cells were tested for the same proliferation, senescence, autophagy, and apoptosis markers that had been tested in NG108-15 cells and found similar results (FIG. 15). All treatments showed an increase in senescence markers, with the most significant being the combination of pantoprazole with NS1643, TMZ, and the combination of NS1643 and TMZ (FIG. 15-A). The size of the nuclei for the combination treatments with the highest levels of senescent cells were also significantly larger than control, confirming this phenotype (FIG. 15-F). p27^{Kip1} levels were investigated and similar changes were observed in NG108-15 cells (FIG. 15-E). All of the treatments with pantoprazole showed an increase in p27^{Kip1} levels, and NS1643 in combination with TMZ showed the highest increase. Cleaved caspase 3 (FIG. 15-D) and LC3-II (FIG. 15-C) levels showed very low non-significant levels of expression indicating that all the cells were not undergoing a high-level of apoptosis or autophagy, respectively. BrdU incorporation decreased in all treatments that showed lower proliferation in FIG. 6-A. These data show that the low proliferation of the U87 cells treated with the most successful drug combinations was driven by increased levels of senescence.

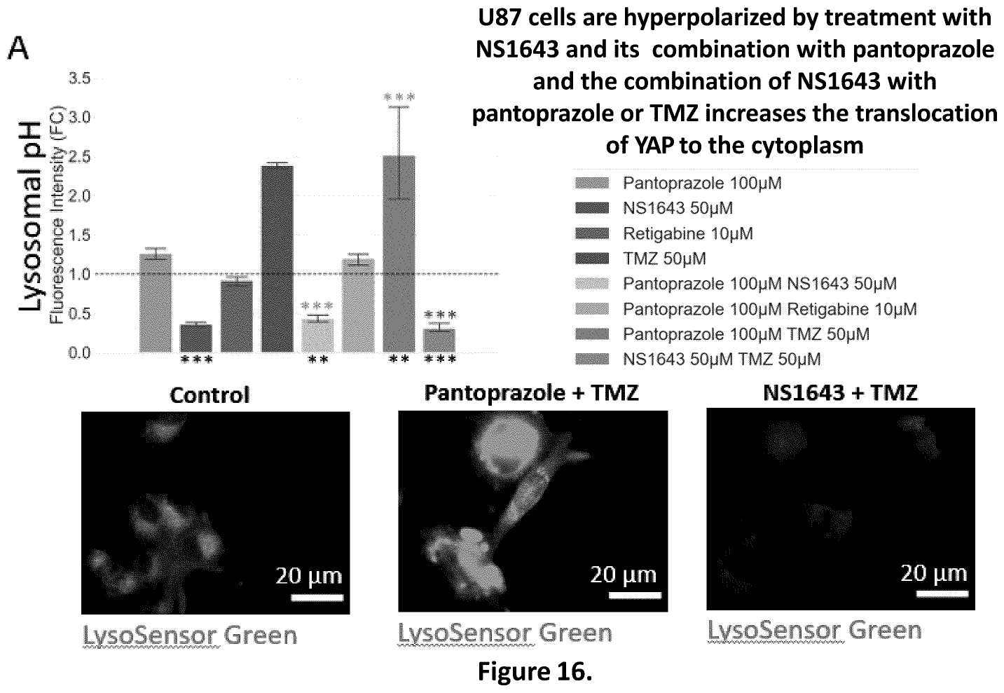

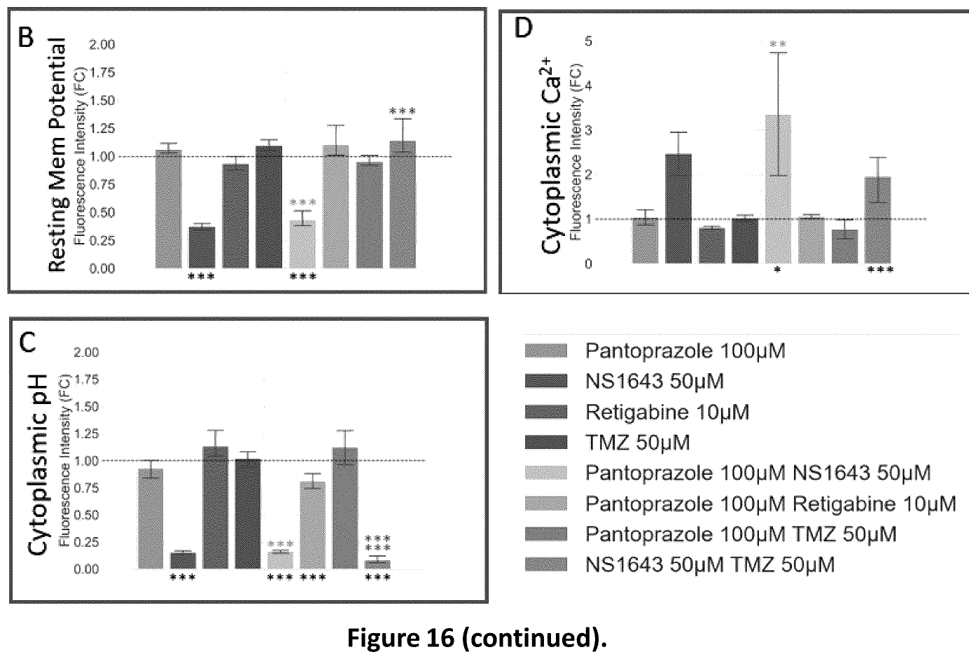

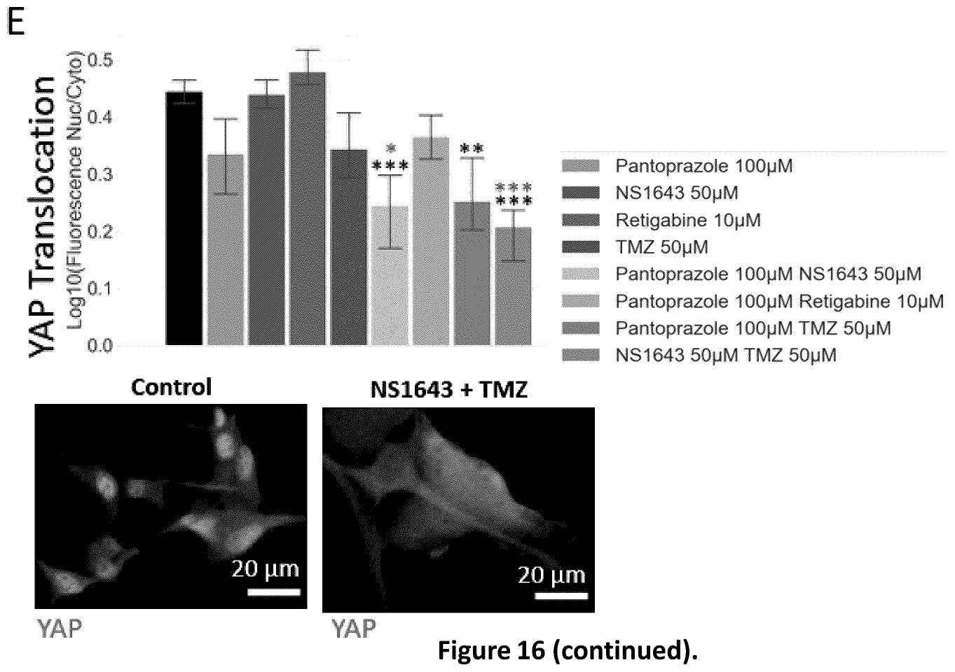

The voltage dye DiBAC4(3) [109,110] was used to measure the resting membrane potential of 6 day treated U87 cells (FIG. 16-B). It was found that NS1643 alone and in combination with pantoprazole showed a significant hyperpolarization as compared to control. To investigate further, pHRodo Green were used to see if the internal pH of the cells was being changed in response to treatment (FIG. 16-C). A dramatic increase in pH was observed for all the treatments that incorporated NS1643, and a slight increase in pH was observed for pantoprazole with retigabine. In addition to the cytoplasmic pH, the lysosomal pH was tested due to reports that pantoprazole de-acidifies the lumen of the lysosome [111,112]. The dye, LysoSensor Green (FIG. 16-A) showed a dramatic alkalization of the lysosomes when treated by NS1643 alone or in combination. Pantoprazole did not show an alkalinization of the lysosome in the U87 cells, which has previously been reported when pantoprazole is delivered in neutral cell culture media at pH 7.4 [113]. In fact, pantoprazole and its combination with TMZ showed a significant increase in lysosomes, which agrees with another study done in neutral pH media [114]. In addition to the dyes, the ratio of cytoplasmic to nuclear YAP was also tested (FIG. 16-E). Pantoprazole has been found to decease YAP activity in ovarian cancer and in the liver [115,116]. Since YAP has been found to be a master regulator of the cell cycle, especially in cancer, the effect of such treatments on this protein have been investigated [116-119]. Pantoprazole treatment alone was found not to have a significant effect on the cytoplasmic to nuclear ratio of YAP but when combined with NS1643 or TMZ it showed a significant decrease indicative of less YAP in the nucleus as compared to the cytoplasm. This significant decrease in the YAP nucleus to cytoplasmic ratio was also evident for the NS1643 in combination with TMZ treatment. Taken together, these data indicate that NS1643 in combination or alone, can increase cytoplasmic calcium levels, increase cytoplasmic pH, and increase lysosomal pH, but a significant decrease in the YAP nuclear to cytoplasmic ratio is only seen when it is in combination with pantoprazole or TMZ.

Neuronal cell toxicity was minimal after a three-day treatment with the top performing drugs and drug combinations. In order to determine whether the effects observed were specific for cancer cells and could be expected to be usable in vivo without harming native neurons, these compounds were tested on human induced pluripotent stem cells derived from fibroblasts and made to commit to a neuronal stem cell lineage. These hiNSCs were differentiated for 7 days in neuronal media and then treated with electroceuticals for 3 days. The short treatment time was necessary in order to be able to perform a Live/Dead assay without too much cell detachment. FIG. 9 shows the percent of cells that died after treatment and compares to control.

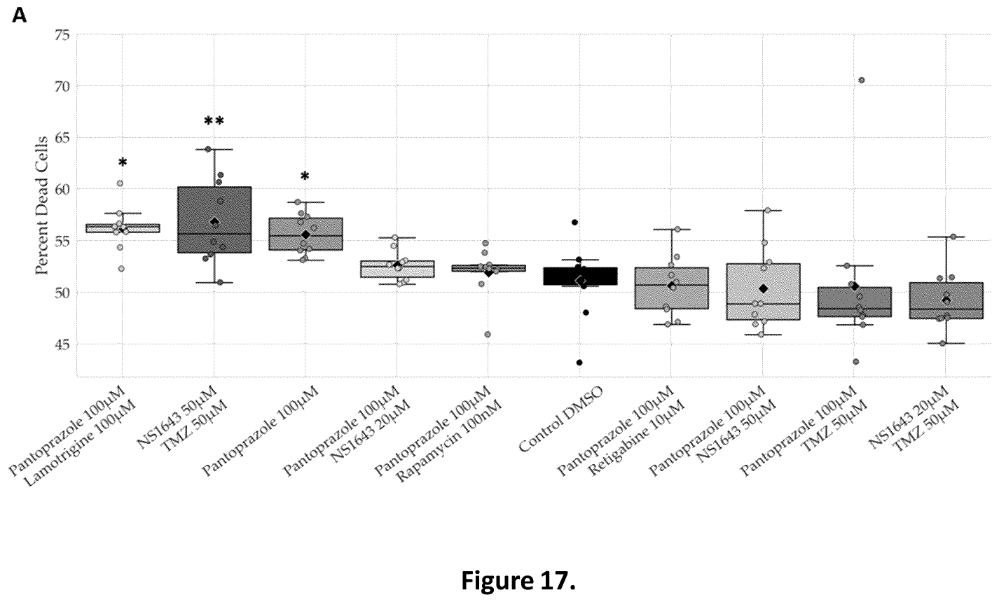

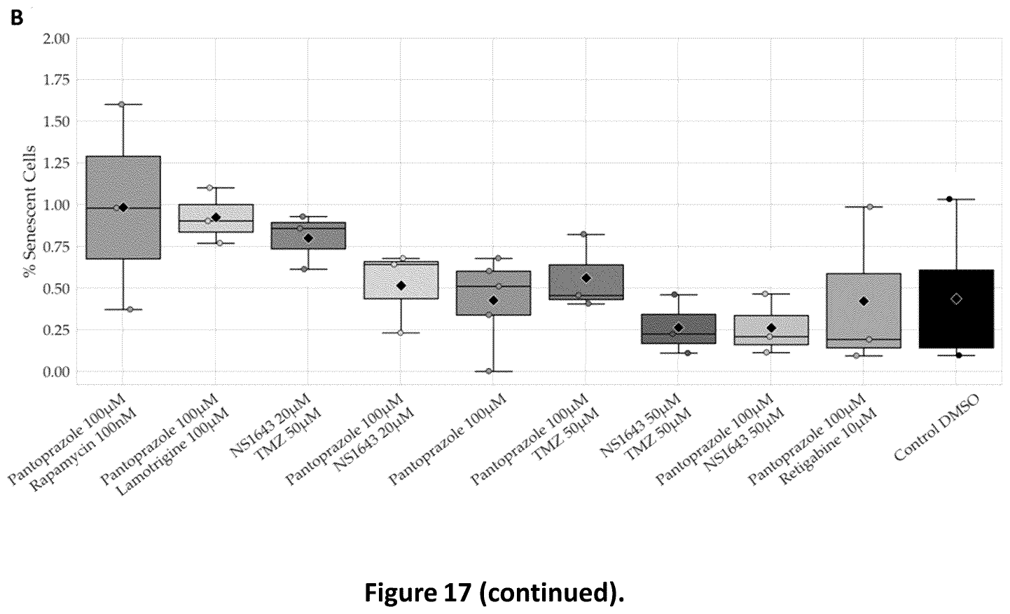

Toxicity analysis by Live/Dead stain in human neurons shows that only three out of 24 treatments showed significant toxicity (FIG. 17-A). Pantoprazole showed a slight increase in toxicity that was significant compared to control, and pantoprazole with lamotrigine also showed a slight increase but was more significant than pantoprazole alone when compared to control. NS1643 at 50 μM in combination with TMZ showed the most toxicity when compared to control. However, the difference between the control toxicity and the most toxic combination of NS1643 at 50 μM and TMZ was still only 5.7% higher than control. A senescence assay was also done on the neurons under all treatments tested in the Live/Dead stain, but no significant differences were found (FIG. 17B), and all senescence levels were all under 1.75%, a stark contrast to the greater than 50% senescence levels in NG108-15 and U87 cells after 6 days of treatment (FIG. 11-A and FIG. 15-A).

### Discussion

Bioelectric drugs. Drugs were selected based on their predicted effects on Vₘₑₘ, which has been shown in amphibian models in vivo to prevent and reverse tumorigenesis and metastatic behavior [120,121]. Indeed, a number of drugs with bioelectric targets, such as ivermectin (a chloride channel drug) [122-125], salinomycin and monensin (ionophores) [122,126], a variety of potassium channel drugs [55,127-131], and drugs targeting proton pumps [132] have been discovered to have anti-cancer activity in various screens [133-137]. Thus, these combinations of compounds represent novel entries to the field of electroceuticals: the repurposing of known ion channel-targeting drugs to manipulate complex cell outcomes [138,139]. This approach has already been used for the design of interventions to repair birth defects of the brain [140,141]. It is likely that a better understanding of the control of cell behavior, alone and in tissues, will enable much more precisely targeted electroceutical interventions in cancer as part of the goal of normalizing cells as an alternative to traditional chemotherapy [142].

Proliferation. The NG108-15 hybrid cell line used in this study shows cancer stem cell characteristics, can be easily transfected and selected, and has been used to study neuronal differentiation for many years. To find treatments that would be robust, all selected compounds were screened in high serum media, which is usually prohibitive for NG108-15 differentiation [83,143,144]. In addition, the high serum media provided an abundance of growth factors that have been shown to be secreted in the peripheral zone of resected GBM tumors and are thought to drive the migration and proliferation of GBM stem cells in the area[9]. The best performing novel combinations were pantoprazole with retigabine, lamotrigine, NS1643, or rapamycin and reduced proliferation when compared to the control by 80%, 85%, 86%, and 90% respectively. FUCCI analysis showed that the cell cycle proportion was dominated by cells in either G1 or early S. All these compounds are already FDA approved for other conditions except for NS1643.

The best performing novel combinations were then used to inform a screen of compounds on a human glioblastoma cell line, U87 (ATCC). Proliferation assays showed 13 drug or drug combinations that reduced proliferation compared to control more significantly than the leading glioblastoma treatment, TMZ. Of these 13, the top four treatments were combinations of pantoprazole or NS1643 with TMZ, and the combination of pantoprazole with retigabine or NS1643, which reduced proliferation when compared to control by 62%, 63%, 71%, and 74%, respectively. FUCCI analysis showed that the cell cycle proportion was again dominated by cells in either G1 or early S.

Differentiation. Differentiation therapy for GBM is an alternative treatment strategy that could possibly overcome the issue of reoccurrence after resection of the tumor [14]. For differentiation therapy to be successful, treatment needs to be effective at clinically relevant concentrations and differentiation needs to be permanent, without cell cycle re-entry after treatment is stopped. This study showed that hyperpolarizing compounds in combination with pantoprazole can be used to arrest the cell cycle of proliferating cells and drive them towards partial differentiation and senescence.

NG108-15 cells treated with the most successful drug combinations showed neuronal, astrocytic, and oligodendrocyte differentiation markers as well as senescence markers. The mixed nature of differentiation markers in NG108-15 cells has been reported previously [145] and might be due to their hybrid neuroblastoma/glioma status. Many of the combinations of drugs tested in NG108-15 cells were also successful in U87 cells and resulted in dramatically less proliferation, less progression through the cell cycle and a significant increase in differentiation markers, which need the cell cycle to be arrested long enough to accumulate [146]. The mixed nature of the differentiation markers that were elevated in U87 cells shows that this line can differentiate into neuron, astrocyte, and oligodendrocyte lineages, in agreement with past studies [147,148]. It is important to note that many of the differentiation markers were heterogeneously expressed and that there is a possibility that in each sample there exist a multitude of differentiated phenotypes instead of just one type. Connexin 43 was also upregulated in treated NG108-15 cells, showing that this marker of cell-to-cell communication was increased as cells went to a more differentiated state as supported by a previous study [97].

Electrophysiology. pH. and cell cycle regulation. NS1643, a hERG channel opener and potassium modulator [149], was shown to hyperpolarize both cell lines that were tested and was one of the most successful single treatments. In combination with pantoprazole, it could arrest the NG108-15 cell cycle long enough to enable differentiation in high serum media and worked well in U87 cells. Treatment with NS1643 in combination with pantoprazole showed a significant effect that was not seen with either treatment alone. Assays in U87 cells showed that this novel combinatorial treatment caused the pH of the cytoplasm and lysosomes to drastically alkalize, and that cytoplasmic calcium increased significantly along with an increase in the cell cycle inhibitor p27^{Kip1} and an increase of senescence-associated beta-galactosidase positive cells. In addition, combination treatments with NS1643 also resulted in a decrease in YAP nuclear to cytoplasmic ratio, elevated levels of which are known to accelerate cancer cell cycle progression, proliferation, therapy resistance, and metastasis [118,119,150]. Thus, both YAP translocation and increased p27^{Kip1} levels were found in the cells that showed the most senescence, confirming what other studies have reported both proteins to be involved in senescence [104,151]. Proliferation under these senescence-inducing treatments was reduced dramatically and U87 cells did not re-enter the cell cycle up to 4 days after treatment was removed. The combination of NS1643 with TMZ was also very effective at reducing proliferation in U87 cells, although according to voltage dye assays it did not hyperpolarize U87 cells. However, the cells did significantly express a variety of differentiation markers after 6 days of treatment. The cells treated with NS1643 and TMZ also showed, like the other NS1643 containing treatments, an increase in lysosomal and cytoplasmic pH, an increase in p27^{kip1}, and a significant decrease in the YAP nuclear to cytoplasmic ratio. It is possible that the voltage dye did not correctly report the membrane potential with this combination due to an interaction with the dye molecule itself, as has been reported for other compounds [152,153].

In agreement with the results herein, NS1643 has also been shown to induce senescence in melanoma and breast cancer [55,127,154]. This senescent phenotype is thought to occur through elevated internal calcium levels (a response to hyperpolarization) which trigger the activation of calcineurin which in turn dephosphorylates NFAT and results in its translocation to the nucleus [131]. In the nucleus, it is possible that translocated NFAT and activated calcineurin, could be increasing the expression of p21^{WAF1/CIP1}, also found caused by NS1643 [131], by a mechanism like that found in differentiating keratinocytes where calcineurin increases Sp1/Sp3-dependent transcription and p21 promoter activity in combination with NFAT [155]. That same study done on keratinocytes showed that calcineurin inactivation resulted in less p27^{kip1} as well [155]. Interestingly, mitochondrial ROS production has been found to be increased by calcineurin activation in neurons [156] and ROS levels were found elevated in breast cancer cells treated with NS1643[127]. Elevated ROS levels have been found to decrease the proteasome function [157] and could possibly increase p27^{kip1} levels through decreased ubiquitination and degradation [158]. Currently, no publications have shown the efficacy of NS1643 on glioblastoma or its efficacy in combination with rapamycin, pantoprazole, or TMZ.

Another successful combination included the KCNQ channel opener and FDA approved epilepsy treatment, retigabine [80]. Although the application of retigabine alone hyperpolarized NG108-15 cells only slightly and reduced proliferation only marginally by itself, its ability to stop NG108-15 proliferation was increased most significantly with pantoprazole. Pantoprazole, a proton-pump inhibitor, worked well on its own at reducing proliferation in these two cell lines. However, when treatment was removed, the cells immediately re-entered the cell cycle, illustrating that treatment with pantoprazole alone does not arrest the cell cycle long enough to allow for terminal differentiation. When pantoprazole was combined with retigabine a synergistic effect was achieved that resulted in less cells re-entering the cell cycle after treatment was removed. Interestingly a depolarization of the NG108-15 cells was observed when they were treated with the combination, but no change was seen in the membrane potential of the U87 cells.

Pantoprazole alone has been reported to increase the alkalinity of the lysosomes by inhibiting V-ATPase channels [79]. However, no alkalization of the lysosomes was observed, and the lysosomal signal was increased. These results agree with a recent study showing that pantoprazole at neutral pH does not inhibit V-ATPase channels and that it instead increases lysosomal biogenesis [113]. Interestingly, that same study showed that pantoprazole interfered with proteosome function, which might explain why it worked so well as a combinatorial treatment. Recent studies have shown that proteosome disfunction along with an increase in p27^{Kip1} leads to senescence and endoreplication, resulting in cells with large nuclei [159]. In addition, the loss of PTEN in U87 (ATCC) cells results in cells that preferentially senesce in response to stress [160]. The presence of endoreplication in strongly senescent cells was evidenced by the size of the nuclei in both cell lines and may explain why the BrdU incorporation results were not as low as the cell count data would suggest they should be. The treatment with pantoprazole alone showed less differentiation and senescence when compared to pantoprazole in combination with retigabine, NS1643, TMZ, or rapamycin in U87 cells. Pantoprazole has been shown to be effective against glioblastoma in vitro [77], but to our knowledge this is the first study that has tested its efficacy in combination with NS1643, lamotrigine, retigabine, rapamycin, or temozolomide.

The leading GBM treatment, TMZ was not significantly effective at reducing proliferation in NG108-15 cells. This has also been observed for some GBM cases as well. However, the combination of TMZ with pantoprazole in NG108-15 cells did significantly lower their proliferation as compared to pantoprazole alone, but did not terminally differentiate them, as evidenced by the recovery assay. However, TMZ by itself was not effective in NG108-15 cells but was significantly effective in U87 cells. This effectiveness in U87 cells might explain why the combination of pantoprazole or NS1643 with TMZ was so effective at reducing proliferation and increasing differentiation markers in these cells and not in the NG108-15 line. It should be noted that these combinations also dramatically increased senescence markers in these cells and caused a significant decrease of the YAP nuclear to cytoplasmic ratio.

Drug Concentrations and FDA Status. The drug concentrations used in these studies were chosen to be close to the Cm, reported for the highest dosages that did not result in unacceptable toxicity. Retigabine, FDA approved for epilepsy, was used at 10 μM, which is close to the reported upper Cₘₐₓ values obtained with 1200 mg/day dose of about 2250 ng/mL or about 7.4 μM [161]. Blood-brain barrier penetrance is good for retigabine with free plasma concentrations being about the same as free brain concentrations [162]. The choice of using high serum to test the drugs and drug combinations also helps in strengthening the clinical relevance of such treatment data because retigabine also has a high plasma protein binding affinity of about 80% [162]. However the binding of a compound in fetal bovine serum can be different than the binding in human serum [163].

Rapamycin, an FDA approved drug for immunosuppression, was used at a dose of 100 nM for immunological studies. This concentration is under the highest dosage of 40 mg/day of a nano-amorphous oral formulation in a fasted state, with a Cₘₐₓ of 219 ng/mL or about 239 nM and toxicity at this level was deemed manageable [164]. Also, rapamycin has been used in a phase 1 clinical trial for glioblastoma and was found to cross the blood brain barrier effectively [165].

Pantoprazole, an FDA approved proton pump inhibitor, was used at a dose of 100 μM. The highest dosage of pantoprazole, given for Zollinger-Ellison syndrome is 240 mg/day and results in a Cmax of 42 mg/L or about 110 μM if the Cmax is proportional to that given for a 30 mg/day dose (as it is for dosages up to 80 mg/day) [166]. However, pantoprazole has poor blood brain barrier penetrance of only 2% [167]. Therefore, its use in glioblastoma therapy will have to rely on novel methods for delivery across the blood-brain barrier, of which there are many new strategies being developed [168]. It should be noted however, that all tests done on pantoprazole in this study were done at a neutral pH, so pantoprazole's efficacy may increase in an in vivo setting where the tumor microenvironment is more acidic [169].

NS1643, a hERG activator, is not currently approved by the FDA. However, it has recently been used in a breast cancer xenograft model in immunodeficient mice and did not show any overt toxicity on the heart or on normal breast epithelial cells [127].

Limitations of the Study. The electrophysiological analysis in NG108 cells showed what immediate changes the compounds had on membrane potential and did not show the changes in potential over time. Pantoprazole did not have a significant effect on the membrane potential of the NG108-15 cells alone, however the effect of pantoprazole on the negation of the hyperpolarization seen with retigabine, lamotrigine, NS1643, rapamycin is extremely interesting considering how quickly it occurred. If pantoprazole was merely blocking the function of those other compounds, then it is curious as to why the combination of the compounds would be more effective than pantoprazole alone. In addition, the lack of hyperpolarization in the NG108-15 cells with the combination treatments points to the fact the hyperpolarization of the membrane potential is not necessarily needed for ion modulating drugs to have an effect. The depolarization of NG108-15 cells seen in the combination of pantoprazole with retigabine or rapamycin could have effects on the ability of the cell cycle to proceed if the needed level of hyperpolarization to proceed through S phase is not reached, an effect seen specifically in OPCs [170-172]. However, this is not the case for the combination of pantoprazole with NS1643 or lamotrigine, which did not have any significant change in resting membrane potential as compared to control in NG108-15 cells.

In this Example, only a DiBAC4(3) based analysis of resting membrane potential was obtained and may have had some interference with the dye caused by the compounds themselves.

Conclusions. The use of the NG108-15 cell line in high serum conditions for the initial screening of many compounds and combinations, provide a subset of drugs that showed a significant decrease in proliferation in the human U87 glioblastoma cell line treated for six days and showed depressed growth up to four days after treatment was removed. These results suggest that treatment will be given in an intermittent manner. The top combinations were capable of terminally differentiating and causing senescence in NG108-15 cells under normally prohibitive conditions, providing robust candidates that showed terminal differentiation and senescence in U87 cells.

### REFERENCES BELOW ARE NUMBERED 1-189 FOR EXAMPLE 2

1. Hanif, F.; Muzaffar, K.; Perveen, K.; Malhi, S. M.; Simjee Sh, U., Glioblastoma Multiforme: A Review of its Epidemiology and Pathogenesis through Clinical Presentation and Treatment. Asian Pac J Cancer Prev 2017, 18 (1), 3-9.
2. Davis, M. E., Glioblastoma: Overview of Disease and Treatment. Clin J Oncol Nurs 2016, 20 (5 Suppl), S2-8.
3. Lemee, J. M.; Clavreul, A.; Aubry, M.; Com, E.; de Tayrac, M.; Eliat, P. A.; Henry, C.; Rousseau, A.; Mosser, J.; Menei, P., Characterizing the peritumoral brain zone in glioblastoma: a multidisciplinary analysis. J Neurooncol 2015, 122 (1), 53-61.
4. Fan, X.; Xiong, Y.; Wang, Y., A reignited debate over the cell(s) of origin for glioblastoma and its clinical implications. Front Med 2019, 13 (5), 531-539.
5. Liu, C.; Sage, J. C.; Miller, M. R.; Verhaak, R. G.; Hippenmeyer, S.; Vogel, H.; Foreman, O.; Bronson, R. T.; Nishiyama, A.; Luo, L.; Zong, H., Mosaic analysis with double markers reveals tumor cell of origin in glioma. Cell 2011, 146 (2), 209-21.
6. Alessandrini, F.; Ceresa, D.; Appolloni, I.; Pagani, F.; Poliani, P. L.; Marubbi, D.; Malatesta, P., Glioblastoma models driven by different mutations converge to the proneural subtype. Cancer Lett 2020, 469, 447-455.
7. Alcantara Llaguno, S. R.; Wang, Z.; Sun, D.; Chen, J.; Xu, J.; Kim, E.; Hatanpaa, K. J.; Raisanen, J. M.; Burns, D. K.; Johnson, J. E.; Parada, L. F., Adult Lineage-Restricted CNS Progenitors Specify Distinct Glioblastoma Subtypes. Cancer Cell 2015, 28 (4), 429-440.
8. Lindberg, N.; Jiang, Y.; Xie, Y.; Bolouri, H.; Kastemar, M.; Olofsson, T.; Holland, E. C.; Uhrbom, L., Oncogenic signaling is dominant to cell of origin and dictates astrocytic or oligodendroglial tumor development from oligodendrocyte precursor cells. J Neurosci 2014, 34 (44), 14644-51.
9. Hide, T.; Shibahara, I.; Kumabe, T., Novel concept of the border niche: glioblastoma cells use oligodendrocytes progenitor cells (GAOs) and microglia to acquire stem cell-like features. Brain Tumor Pathol 2019, 36 (2), 63-73.
10. Yan, M.; Liu, Q., Differentiation therapy: a promising strategy for cancer treatment. Chin J Cancer 2016, 35, 3.
11. Levin, M.; Pezzulo, G.; Finkelstein, J. M., Endogenous Bioelectric Signaling Networks: Exploiting Voltage Gradients for Control of Growth and Form. Annu Rev Biomed Eng 2017, 19, 353-387.
12. Panicker, S. P.; Raychaudhuri, B.; Sharma, P.; Tipps, R.; Mazumdar, T.; Mal, A. K.; Palomo, J. M.; Vogelbaum, M. A.; Haque, S. J., p300- and Myc-mediated regulation of glioblastoma multiforme cell differentiation. Oncotarget 2010, 1 (4), 289-303.
13. Heo, J. C.; Jung, T. H.; Lee, S.; Kim, H. Y.; Choi, G.; Jung, M.; Jung, D.; Lee, H. K.; Lee, J. O.; Park, J. H.; Hwang, D.; Seol, H. J.; Cho, H., Effect of bexarotene on differentiation of glioblastoma multiforme compared with ATRA. Clin Exp Metastasis 2016, 33 (5), 417-29.
14. Kang, T. W.; Choi, S. W.; Yang, S. R.; Shin, T. H.; Kim, H. S.; Yu, K. R.; Hong, I. S.; Ro, S.; Cho, J. M.; Kang, K. S., Growth arrest and forced differentiation of human primary glioblastoma multiforme by a novel small molecule. Sci Rep 2014, 4, 5546.
15. Xing, F.; Luan, Y.; Cai, J.; Wu, S.; Mai, J.; Gu, J.; Zhang, H.; Li, K.; Lin, Y.; Xiao, X.; Liang, J.; Li, Y.; Chen, W.; Tan, Y.; Sheng, L.; Lu, B.; Lu, W.; Gao, M.; Qiu, P.; Su, X.; Yin, W.; Hu, J.; Chen, Z.; Sai, K.; Wang, J.; Chen, F.; Chen, Y.; Zhu, S.; Liu, D.; Cheng, S.; Xie, Z.; Zhu, W.; Yan, G., The Anti-Warburg Effect Elicited by the cAMP-PGC1alpha Pathway Drives Differentiation of Glioblastoma Cells into Astrocytes. Cell Rep 2017, 18 (2), 468-481.
16. Piccirillo, S. G.; Reynolds, B. A.; Zanetti, N.; Lamorte, G.; Binda, E.; Broggi, G.; Brem, H.; Olivi, A.; Dimeco, F.; Vescovi, A. L., Bone morphogenetic proteins inhibit the tumorigenic potential of human brain tumour-initiating cells. Nature 2006, 444 (7120), 761-5.
17. Park, D. M.; Li, J.; Okamoto, H.; Akeju, O.; Kim, S. H.; Lubensky, I.; Vortmeyer, A.; Dambrosia, J.; Weil, R. J.; Oldfield, E. H.; Park, J. K.; Zhuang, Z., N-CoR pathway targeting induces glioblastoma derived cancer stem cell differentiation. Cell Cycle 2007, 6 (4), 467-70.
18. Dong, Y.; Han, Q.; Zou, Y.; Deng, Z.; Lu, X.; Wang, X.; Zhang, W.; Jin, H.; Su, J.; Jiang, T.; Ren, H., Long-term exposure to imatinib reduced cancer stem cell ability through induction of cell differentiation via activation of MAPK signaling in glioblastoma cells. Mol Cell Biochem 2012, 370 (1-2), 89-102.
19. Chlapek, P.; Slavikova, V.; Mazanek, P.; Sterba, J.; Veselska, R., Why Differentiation Therapy Sometimes Fails: Molecular Mechanisms of Resistance to Retinoids. Int J Mol Sci 2018, 19 (1).
20. Mathews, J.; Levin, M., The body electric 2.0: recent advances in developmental bioelectricity for regenerative and synthetic bioengineering. Curr Opin Biotechnol 2018, 52, 134-144.
21. Bates, E., Ion channels in development and cancer. Annu Rev Cell Dev Biol 2015, 31, 231-47.
22. Levin, M., Bioelectric signaling: Reprogrammable circuits underlying embryogenesis, regeneration, and cancer. Cell 2021, 184 (4), 1971-1989.
23. Srivastava, P.; Kane, A.; Harrison, C.; Levin, M., A Meta-Analysis of Bioelectric Data in Cancer, Embryogenesis, and Regeneration. Bioelectricity 2020, in press.
24. Rao, V. R.; Perez-Neut, M.; Kaja, S.; Gentile, S., Voltage-gated ion channels in cancer cell proliferation. Cancers (Basel) 2015, 7(2), 849-75.
25. Arcangeli, A.; Becchetti, A., New Trends in Cancer Therapy: Targeting Ion Channels and Transporters. Pharmaceuticals (Basel) 2010, 3 (4), 1202-1224.
26. Litan, A.; Langhans, S. A., Cancer as a channelopathy: ion channels and pumps in tumor development and progression. Front Cell Neurosci 2015, 9, 86.
27. Rapetti-Mauss, R.; Berenguier, C.; Allegrini, B.; Soriani, O., Interplay Between Ion Channels and the Wnt/beta-Catenin Signaling Pathway in Cancers. Front Pharmacol 2020, 11, 525020.
28. Dowd, J.; Hendin, J.; Fukushiro-Lopes, D. F.; Laczynski, D.; Gentile, S., Ion Channels in Breast Cancer: From Signaling to Therapy. InTech: 2017.
29. Lang, F.; Stournaras, C., Ion channels in cancer: future perspectives and clinical potential. Philos Trans R Soc Lond B Biol Sci 2014, 369 (1638), 20130108.
30. Yang, M.; Brackenbury, W. J., Membrane potential and cancer progression. Front Physiol 2013, 4, 185.
31. Cheng, Q.; Chen, A.; Du, Q.; Liao, Q.; Shuai, Z.; Chen, C.; Yang, X.; Hu, Y.; Zhao, J.; Liu, S.; Wen, G. R.; An, J.; Jing, H.; Tuo, B.; Xie, R.; Xu, J., Novel insights into ion channels in cancer stem cells (Review). Int J Oncol 2018, 53 (4), 1435-1441.
32. Leanza, L.; Manago, A.; Zoratti, M.; Gulbins, E.; Szabo, I., Pharmacological targeting of ion channels for cancer therapy: In vivo evidences. Biochim Biophys Acta 2016, 1863 (6 Pt B), 1385-97.
33. Lobikin, M.; Chernet, B.; Lobo, D.; Levin, M., Resting potential, oncogene-induced tumorigenesis, and metastasis: the bioelectric basis of cancer in vivo. Phys Biol 2012, 9 (6), 065002.
34. Becchetti, A.; Munaron, L.; Arcangeli, A., The role of ion channels and transporters in cell proliferation and cancer. Front Physiol 2013, 4, 312.
35. Sontheimer, H., An unexpected role for ion channels in brain tumor metastasis. Exp Biol Med (Maywood) 2008, 233 (7), 779-91.
36. Binggeli, R.; Weinstein, R. C., Membrane potentials and sodium channels: Hypotheses for growth regulation and cancer formation based on changes in sodium channels and gap junctions. Journal of Theoretical Biology 1986, 123 (4), 377-401.
37. Srivastava, P.; Kane, A.; Harrison, C.; Levin, M., A Meta-Analysis of Bioelectric Data in Cancer, Embryogenesis, and Regeneration. Bioelectricity 2020.
38. Chernet, B. T.; Levin, M., Transmembrane voltage potential is an essential cellular parameter for the detection and control of tumor development in a Xenopus model. Dis Model Mech 2013, 6 (3), 595-607.
39. Cone, C. D., Jr.; Tongier, M., Jr., Control of somatic cell mitosis by simulated changes in the transmembrane potential level. Oncology 1971, 25 (2), 168-82.
40. Stillwell, E. F.; Cone, C. M.; Cone, C. D., Jr., Stimulation of DNA synthesis in CNS neurones by sustained depolarisation. Nat New Biol 1973, 246 (152), 110-1.
41. Cone, C. D., Unified theory on the basic mechanism of normal mitotic control and oncogenesis. Journal of Theoretical Biology 1971, 30 (1), 151-181.
42. Sundelacruz, S.; Levin, M.; Kaplan, D. L., Depolarization alters phenotype, maintains plasticity of predifferentiated mesenchymal stem cells. Tissue Eng Part A 2013, 19 (17-18), 1889-908.
43. Sundelacruz, S.; Levin, M.; Kaplan, D. L., Membrane potential controls adipogenic and osteogenic differentiation of mesenchymal stem cells. PLoS One 2008, 3 (11), e3737.
44. Zhang, Y. Y.; Yue, J.; Che, H.; Sun, H. Y.; Tse, H. F.; Li, G. R., BKCa and hEag1 channels regulate cell proliferation and differentiation in human bone marrow-derived mesenchymal stem cells. J Cell Physiol 2014, 229 (2), 202-12.
45. Meszaros, B.; Papp, F.; Mocsar, G.; Kokai, E.; Kovacs, K.; Tajti, G.; Panyi, G., The voltage-gated proton channel hHv1 is functionally expressed in human chorion-derived mesenchymal stem cells. Sci Rep 2020, 10 (1), 7100.
46. Pillozzi, S.; Becchetti, A., Ion Channels in Hematopoietic and Mesenchymal Stem Cells. Stem Cells Int 2012, 2012.
47. Chernet, B. T.; Adams, D. S.; Lobikin, M.; Levin, M., Use of genetically encoded, light-gated ion translocators to control tumorigenesis. Oncotarget 2016, 7 (15), 19575-88.
48. Chernet, B.; Levin, M., Endogenous Voltage Potentials and the Microenvironment: Bioelectric Signals that Reveal, Induce and Normalize Cancer. J Clin Exp Oncol 2013, Suppl 1.
49. Blackiston, D. J.; McLaughlin, K. A.; Levin, M., Bioelectric controls of cell proliferation: ion channels, membrane voltage and the cell cycle. Cell Cycle 2009, 8 (21), 3527-36.
50. Payne, S. L.; Levin, M.; Oudin, M. J., Bioelectric Control of Metastasis in Solid Tumors. Bioelectricity 2019, 1 (3), 114-130.
51. Ruggieri, P.; Mangino, G.; Fioretti, B.; Catacuzzeno, L.; Puca, R.; Ponti, D.; Miscusi, M.; Franciolini, F.; Ragona, G.; Calogero, A., The inhibition of KCa3.1 channels activity reduces cell motility in glioblastoma derived cancer stem cells. PLoS One 2012, 7(10), e47825.
52. Comes, N.; Serrano-Albarras, A.; Capera, J.; Serrano-Novillo, C.; Condom, E.; Ramon, Y. C. S.; Ferreres, J. C.; Felipe, A., Involvement of potassium channels in the progression of cancer to a more malignant phenotype. Biochim Biophys Acta 2015, 1848 (10 Pt B), 2477-92.
53. Pollak, J.; Rai, K. G.; Funk, C. C.; Arora, S.; Lee, E.; Zhu, J.; Price, N. D.; Paddison, P. J.; Ramirez, J. M.; Rostomily, R. C., Ion channel expression patterns in glioblastoma stem cells with functional and therapeutic implications for malignancy. PLoS One 2017, 12 (3), e0172884.
54. Jehle, J.; Schweizer, P. A.; Katus, H. A.; Thomas, D., Novel roles for hERG K(+) channels in cell proliferation and apoptosis. Cell Death Dis 2011, 2, e193.
55. Breuer, E. K.; Fukushiro-Lopes, D.; Dalheim, A.; Burnette, M.; Zartman, J.; Kaja, S.; Wells, C.; Campo, L.; Curtis, K. J.; Romero-Moreno, R.; Littlepage, L. E.; Niebur, G. L.; Hoskins, K.; Nishimura, M. I.; Gentile, S., Potassium channel activity controls breast cancer metastasis by affecting beta-catenin signaling. Cell Death Dis 2019, 10 (3), 180.
56. Terrasi, A.; Bertolini, I.; Martelli, C.; Gaudioso, G.; Di Cristofori, A.; Storaci, A. M.; Formica, M.; Bosari, S.; Caroli, M.; Ottobrini, L.; Vaccari, T.; Vaira, V., Specific V-ATPase expression sub-classifies IDHwt lower-grade gliomas and impacts glioma growth in vivo. EBioMedicine 2019, 41, 214-224.
57. Yekula, A.; Yekula, A.; Muralidharan, K.; Kang, K.; Carter, B. S.; Balaj, L., Extracellular Vesicles in Glioblastoma Tumor Microenvironment. Front Immunol 2019, 10, 3137.
58. Harguindey, S.; Polo Orozco, J.; Alfarouk, K. O.; Devesa, J., Hydrogen Ion Dynamics of Cancer and a New Molecular, Biochemical and Metabolic Approach to the Etiopathogenesis and Treatment of Brain Malignancies. Int J Mol Sci 2019, 20 (17).
59. Hamprecht, B.; Glaser, T.; Reiser, G.; Bayer, E.; Propst, F., Culture and characteristics of hormone-responsive neuroblastoma X glioma hybrid cells. Methods Enzymol 1985, 109, 316-41.
60. Lichtshtein, D.; Dunlop, K.; Kaback, H. R.; Blume, A. J., Mechanism of monensin-induced hyperpolarization of neuroblastoma-glioma hybrid NG108-15. Proc Natl Acad Sci USA 1979, 76 (6), 2580-4.
61. Mitchell, P. J.; Hanson, J. C.; Quets-Nguyen, A. T.; Bergeron, M.; Smith, R. C., A quantitative method for analysis of in vitro neurite outgrowth. J Neurosci Methods 2007, 164 (2), 350-62.
62. Pancrazio, J. J.; Ma, W.; Grant, G. M.; Shaffer, K. M.; Kao, W. Y.; Liu, Q.-Y.; Manos, P.; Barker, J. L.; Stenger, D. A., A role for inwardly rectifying K+ channels in differentiation of NG108-15 neuroblastoma glioma cells. Journal of Neurobiology 1999, 38 (4), 466-474.
63. Lee, J.; Kotliarova, S.; Kotliarov, Y.; Li, A.; Su, Q.; Donin, N. M.; Pastorino, S.; Purow, B. W.; Christopher, N.; Zhang, W.; Park, J. K.; Fine, H. A., Tumor stem cells derived from glioblastomas cultured in bFGF and EGF more closely mirror the phenotype and genotype of primary tumors than do serum-cultured cell lines. Cancer Cell 2006, 9 (5), 391-403.
64. Ledur, P. F.; Onzi, G. R.; Zong, H.; Lenz, G., Culture conditions defining glioblastoma cells behavior: what is the impact for novel discoveries?Oncotarget 2017, 8 (40), 69185-69197.
65. Hong, X.; Chedid, K.; Kalkanis, S. N., Glioblastoma cell line-derived spheres in serumcontaining medium versus serum-free medium: a comparison of cancer stem cell properties. Int J Oncol 2012, 41 (5), 1693-700.
66. Haspels, H. N.; Rahman, M. A.; Joseph, J. V.; Gras Navarro, A.; Chekenya, M., Glioblastoma Stem-Like Cells Are More Susceptible Than Differentiated Cells to Natural Killer Cell Lysis Mediated Through Killer Immunoglobulin-Like Receptors-Human Leukocyte Antigen Ligand Mismatch and Activation Receptor-Ligand Interactions. Front Immunol 2018, 9, 1345.
67. Sladitschek, H. L.; Neveu, P. A., MXS-Chaining: A Highly Efficient Cloning Platform for Imaging and Flow Cytometry Approaches in Mammalian Systems. PLoS One 2015, 10 (4), e0124958.
68. Cairns, D. M.; Chwalek, K.; Moore, Y. E.; Kelley, M. R.; Abbott, R. D.; Moss, S.; Kaplan, D. L., Expandable and Rapidly Differentiating Human Induced Neural Stem Cell Lines for Multiple Tissue Engineering Applications. Stem Cell Reports 2016, 7(3), 557-570.
69. Goedhart, J.; von Stetten, D.; Noirclerc-Savoye, M.; Lelimousin, M.; Joosen, L.; Hink, M. A.; van Weeren, L.; Gadella, T. W., Jr.; Royant, A., Structure-guided evolution of cyan fluorescent proteins towards a quantum yield of 93%. Nat Commun 2012, 3, 751.
70. Marh, J.; Stoytcheva, Z.; Urschitz, J.; Sugawara, A.; Yamashiro, H.; Owens, J. B.; Stoytchev, I.; Pelczar, P.; Yanagimachi, R.; Moisyadi, S., Hyperactive self-inactivating piggyBac for transposase-enhanced pronuclear microinjection transgenesis. Proc Natl Acad Sci USA 2012, 109 (47), 19184-9.
71. Owens, J. B.; Mathews, J.; Davy, P.; Stoytchev, I.; Moisyadi, S.; Allsopp, R., Effective Targeted Gene Knockdown in Mammalian Cells Using the piggyBac Transposase-based Delivery System. Mol Ther Nucleic Acids 2013, 2, e137.
72. Kamentsky, L.; Jones, T. R.; Fraser, A.; Bray, M.-A.; Logan, D. J.; Madden, K. L.; Ljosa, V.; Rueden, C.; Eliceiri, K. W.; Carpenter, A. E., Improved structure, function and compatibility for CellProfiler: modular high-throughput image analysis software. Bioinformatics 2011, 27 (8), 1179-1180.
73. Sero, J. E.; Bakal, C., Multiparametric Analysis of Cell Shape Demonstrates that beta-PIX Directly Couples YAP Activation to Extracellular Matrix Adhesion. Cell Syst 2017, 4 (1), 84-96 e6.
74. Pan, J. Q.; Baez-Nieto, D.; Allen, A.; Wang, H. R.; Cottrell, J. R., Developing High-Throughput Assays to Analyze and Screen Electrophysiological Phenotypes. Methods Mol Biol 2018, 1787, 235-252.
75. Chinnaiyan, P.; Won, M.; Wen, P. Y.; Rojiani, A. M.; Werner-Wasik, M.; Shih, H. A.; Ashby, L. S.; Michael Yu, H. H.; Stieber, V. W.; Malone, S. C.; Fiveash, J. B.; Mohile, N. A.; Ahluwalia, M. S.; Wendland, M. M.; Stella, P. J.; Kee, A. Y.; Mehta, M. P., A randomized phase II study of everolimus in combination with chemoradiation in newly diagnosed glioblastoma: results of NRG Oncology RTOG 0913. Neuro Oncol 2018, 20 (5), 666-673.
76. Fouladi, M.; Laningham, F.; Wu, J.; O'Shaughnessy, M. A.; Molina, K.; Broniscer, A.; Spunt, S. L.; Luckett, I.; Stewart, C. F.; Houghton, P. J.; Gilbertson, R. J.; Furman, W. L., Phase I study of everolimus in pediatric patients with refractory solid tumors. J Clin Oncol 2007, 25 (30), 4806-12.
77. Geeviman, K.; Babu, D.; Prakash Babu, P., Pantoprazole Induces Mitochondrial Apoptosis and Attenuates NF-kappaB Signaling in Glioma Cells. Cell Mol Neurobiol 2018, 38 (8), 1491-1504.
78. Beier, D.; Schriefer, B.; Brawanski, K.; Hau, P.; Weis, J.; Schulz, J. B.; Beier, C. P., Efficacy of clinically relevant temozolomide dosing schemes in glioblastoma cancer stem cell lines. J Neurooncol 2012, 109 (1), 45-52.
79. Shen, W.; Zou, X.; Chen, M.; Shen, Y.; Huang, S.; Guo, H.; Zhang, L.; Liu, P., Effect of pantoprazole on human gastric adenocarcinoma SGC7901 cells through regulation of phospho-LRP6 expression in Wnt/β-catenin signaling. Oncology Reports 2013, 30 (2), 851-855.
80. Gunthorpe, M. J.; Large, C. H.; Sankar, R., The mechanism of action of retigabine (ezogabine), a first-in-class K+ channel opener for the treatment of epilepsy. Epilepsia 2012, 53 (3), 412-24.
81. Lagrange, A., Retigabine: bending potassium channels to our will. Epilepsy Curr 2005, 5 (5), 166-8.
82. Nakatani, Y.; Masuko, H.; Amano, T., Effect of lamotrigine on Na(v)1.4 voltage-gated sodium channels. J Pharmacol Sci 2013, 123 (2), 203-6.
83. Chin, T. Y.; Kao, C. H.; Wang, H. Y.; Huang, W. P.; Ma, K. H.; Chueh, S. H., Inhibition of the mammalian target of rapamycin promotes cyclic AMP-induced differentiation of NG108-15 cells. Autophagy 2010, 6 (8), 1139-56.
84. Raker, V. K.; Becker, C.; Steinbrink, K., The cAMP Pathway as Therapeutic Target in Autoimmune and Inflammatory Diseases. Front Immunol 2016, 7, 123.
85. Lee, S. Y., Temozolomide resistance in glioblastoma multiforme. Genes Dis 2016, 3 (3), 198-210.
86. Yeh, P. S.; Wu, S. J.; Hung, T. Y.; Huang, Y. M.; Hsu, C. W.; Sze, C. I.; Hsieh, Y. J.; Huang, C. W.; Wu, S. N., Evidence for the Inhibition by Temozolomide, an Imidazotetrazine Family Alkylator, of Intermediate-Conductance Ca2+-Activated K+ Channels in Glioma Cells. Cell Physiol Biochem 2016, 38 (5), 1727-42.
87. Soltani, M. H.; Pichardo, R.; Song, Z.; Sangha, N.; Camacho, F.; Satyamoorthy, K.; Sangueza, O. P.; Setaluri, V., Microtubule-associated protein 2, a marker of neuronal differentiation, induces mitotic defects, inhibits growth of melanoma cells, and predicts metastatic potential of cutaneous melanoma. Am J Pathol 2005, 166 (6), 1841-50.
88. Sabelstrom, H.; Petri, R.; Shchors, K.; Jandial, R.; Schmidt, C.; Sacheva, R.; Masic, S.; Yuan, E.; Fenster, T.; Martinez, M.; Saxena, S.; Nicolaides, T. P.; Ilkhanizadeh, S.; Berger, M. S.; Snyder, E. Y.; Weiss, W. A.; Jakobsson, J.; Persson, A. I., Driving Neuronal Differentiation through Reversal of an ERK1/2-miR-124-SOX9 Axis Abrogates Glioblastoma Aggressiveness. Cell Rep 2019, 28 (8), 2064-2079 el1.
89. Yan, T.; Skaftnesmo, K. O.; Leiss, L.; Sleire, L.; Wang, J.; Li, X.; Enger, P. O., Neuronal markers are expressed in human gliomas and NSE knockdown sensitizes glioblastoma cells to radiotherapy and temozolomide. Bmc Cancer 2011, 11, 524.
90. Yuan, A.; Rao, M. V.; Veeranna; Nixon, R. A., Neurofilaments and Neurofilament Proteins in Health and Disease. Cold Spring Harb Perspect Biol 2017, 9 (4).
91. Du, J.; Yi, M.; Zhou, F.; He, W.; Yang, A.; Qiu, M.; Huang, H., S100B is selectively expressed by gray matter protoplasmic astrocytes and myelinating oligodendrocytes in the developing CNS. Mol Brain 2021, 14 (1), 154.
92. Yang, Z.; Wang, K. K., Glial fibrillary acidic protein: from intermediate filament assembly and gliosis to neurobiomarker. Trends Neurosci 2015, 38 (6), 364-74.
93. Landeira, B. S.; Santana, T.; Araujo, J. A. M.; Tabet, E. I.; Tannous, B. A.; Schroeder, T.; Costa, M. R., Activity-Independent Effects of CREB on Neuronal Survival and Differentiation during Mouse Cerebral Cortex Development. Cereb Cortex 2018, 28 (2), 538-548.
94. Li, Y.; Yin, W.; Wang, X.; Zhu, W.; Huang, Y.; Yan, G., Cholera toxin induces malignant glioma cell differentiation via the PKA/CREB pathway. Proc Natl Acad Sci USA 2007, 104 (33), 13438-43.
95. Shiga, H.; Yamane, Y.; Kubo, M.; Sakurai, Y.; Asou, H.; Ito, E., Differentiation of immature oligodendrocytes is regulated by phosphorylation of cyclic AMP-response element binding protein by a protein kinase C signaling cascade. J Neurosci Res 2005, 80 (6), 767-76.
96. Bender, R. A.; Lauterborn, J. C.; Gall, C. M.; Cariaga, W.; Baram, T. Z., Enhanced CREB phosphorylation in immature dentate gyrus granule cells precedes neurotrophin expression and indicates a specific role of CREB in granule cell differentiation. Eur J Neurosci 2001, 13 (4), 679-86.
97. Hitomi, M.; Deleyrolle, L. P.; Mulkearns-Hubert, E. E.; Jarrar, A.; Li, M.; Sinyuk, M.; Otvos, B.; Brunet, S.; Flavahan, W. A.; Hubert, C. G.; Goan, W.; Hale, J. S.; Alvarado, A. G.; Zhang, A.; Rohaus, M.; Oli, M.; Vedam-Mai, V.; Fortin, J. M.; Futch, H. S.; Griffith, B.; Wu, Q.; Xia, C. H.; Gong, X.; Ahluwalia, M. S.; Rich, J. N.; Reynolds, B. A.; Lathia, J. D., Differential connexin function enhances self-renewal in glioblastoma. Cell Rep 2015, 11 (7), 1031-42.
98. Lee, B. Y.; Han, J. A.; Im, J. S.; Morrone, A.; Johung, K.; Goodwin, E. C.; Kleijer, W. J.; DiMaio, D.; Hwang, E. S., Senescence-associated beta-galactosidase is lysosomal beta-galactosidase. Aging Cell 2006, 5 (2), 187-95.
99. Tang, X. M.; Beesley, J. S.; Grinspan, J. B.; Seth, P.; Kamholz, J.; Cambi, F., Cell Cycle Arrest Induced by Ectopic Expression of p27 Is Not Sufficient to Promote Oligodendrocyte Differentiation. Journal of Cellular Biochemistry 1999, 76 (2), 270-279.
100. Schiappacassi, M.; Lovat, F.; Canzonieri, V.; Belletti, B.; Berton, S.; Di Stefano, D.; Vecchione, A.; Colombatti, A.; Baldassarre, G., p27Kip1 expression inhibits glioblastoma growth, invasion, and tumor-induced neoangiogenesis. Molecular Cancer Therapeutics 2008, 7 (5), 1164-1175.
101. Lloyd, R. V.; Erickson, L. A.; Jin, L.; Kulig, E.; Qian, X.; Cheville, J. C.; Scheithauer, B. W., p27kip1: a multifunctional cyclin-dependent kinase inhibitor with prognostic significance in human cancers. Am J Pathol 1999, 154 (2), 313-23.
102. McKay, L. K.; White, J. P., The AMPK/p27(Kip1) Pathway as a Novel Target to Promote Autophagy and Resilience in Aged Cells. Cells 2021, 10 (6).
103. Love, R., Cyclin D1 and P27KIP1: The Gatekeepers of Dysplasia. Journal of Immunological Sciences 2018, 2 (3), 30-39.
104. Collado, M.; Medema, R. H.; Garcia-Cao, I.; Dubuisson, M. L.; Barradas, M.; Glassford, J.; Rivas, C.; Burgering, B. M.; Serrano, M.; Lam, E. W., Inhibition of the phosphoinositide 3-kinase pathway induces a senescence-like arrest mediated by p27Kip1. J Biol Chem 2000, 275 (29), 21960-8.
105. Tsai, C. H.; Chang, C. Y.; Lin, B. Z.; Wu, Y. L.; Wu, M. H.; Lin, L. T.; Huang, W. C.; Holz, J. D.; Sheu, T. J.; Lee, J. S.; Kitsis, R. N.; Tai, P. H.; Lee, Y. J., Up-regulation of cofilin-1 in cell senescence associates with morphological change and p27(kip1)-mediated growth delay. Aging Cell 2021, 20 (1), e13288.
106. Kawamata, S.; Sakaida, H.; Hori, T.; Maeda, M.; Uchiyama, T., The upregulation of p27Kip1 by rapamycin results in G1 arrest in exponentially growing T-cell lines. Blood 1998, 91 (2), 561-9.
107. O'Leary, L. A.; Davoli, M. A.; Belliveau, C.; Tanti, A.; Ma, J. C.; Farmer, W. T.; Turecki, G.; Murai, K. K.; Mechawar, N., Characterization of Vimentin-Immunoreactive Astrocytes in the Human Brain. Front Neuroanat 2020, 14, 31.
108. Deng, Z.; Du, W. W.; Fang, L.; Shan, S. W.; Qian, J.; Lin, J.; Qian, W.; Ma, J.; Rutnam, Z. J.; Yang, B. B., The intermediate filament vimentin mediates microRNA miR-378 function in cellular self-renewal by regulating the expression of the Sox2 transcription factor. J Biol Chem 2013, 288 (1), 319-31.
109. Adams, D. S.; Levin, M., Measuring resting membrane potential using the fluorescent voltage reporters DiBAC4(3) and CC2-DMPE. Cold Spring Harbor protocols 2012, 2012 (4), 459-64.
110. Adams, D. S.; Levin, M., General principles for measuring resting membrane potential and ion concentration using fluorescent bioelectricity reporters. Cold Spring Harbor protocols 2012, 2012 (4), 385-97.
111. Lu, Z. N.; Shi, Z. Y.; Dang, Y. F.; Cheng, Y. N.; Guan, Y. H.; Hao, Z. J.; Tian, B.; He, H. W.; Guo, X. L., Pantoprazole pretreatment elevates sensitivity to vincristine in drug-resistant oral epidermoid carcinoma in vitro and in vivo. Biomed Pharmacother 2019, 120, 109478.
112. Liu, W.; Baker, S. S.; Trinidad, J.; Burlingame, A. L.; Baker, R. D.; Forte, J. G.; Virtuoso, L. P.; Egilmez, N. K.; Zhu, L., Inhibition of lysosomal enzyme activities by proton pump inhibitors. J Gastroenterol 2013, 48 (12), 1343-52.
113. Cao, Y.; Chen, M.; Tang, D. H.; Yan, H. L.; Ding, X. W.; Zhou, F.; Zhang, M. M.; Xu, G. F.; Zhang, W. J.; Zhang, S.; Zhuge, Y. Z.; Wang, L.; Zou, X. P., The proton pump inhibitor pantoprazole disrupts protein degradation systems and sensitizes cancer cells to death under various stresses. Cell Death & Disease 2018, 9.
114. Sachs, G.; Shin, J. M., The basis of differentiation of PPIs. Drugs Today (Barc) 2004, 40 Suppl A, 9-14.
115. Lu, Z. N.; Niu, W. X.; Zhang, N.; Ge, M. X.; Bao, Y. Y.; Ren, Y.; Guo, X. L.; He, H. W., Pantoprazole ameliorates liver fibrosis and suppresses hepatic stellate cell activation in bile duct ligation rats by promoting YAP degradation. Acta Pharmacol Sin 2021.
116. He, J.; Shi, X. Y.; Li, Z. M.; Pan, X. H.; Li, Z. L.; Chen, Y.; Yan, S. J.; Xiao, L., Proton pump inhibitors can reverse the YAP mediated paclitaxel resistance in epithelial ovarian cancer. BMC Mol Cell Biol 2019, 20 (1), 49.
117. Lian, I.; Kim, J.; Okazawa, H.; Zhao, J.; Zhao, B.; Yu, J.; Chinnaiyan, A.; Israel, M. A.; Goldstein, L. S.; Abujarour, R.; Ding, S.; Guan, K. L., The role of YAP transcription coactivator in regulating stem cell self-renewal and differentiation. Genes Dev 2010, 24 (11), 1106-18.
118. Zou, R.; Xu, Y. H.; Feng, Y. F.; Shen, M. Q.; Yuan, F.; Yuan, Y. Z., YAP nuclear-cytoplasmic translocation is regulated by mechanical signaling, protein modification, and metabolism. Cell Biol Int 2020, 44 (7), 1416-1425.
119. Zanconato, F.; Cordenonsi, M.; Piccolo, S., YAP/TAZ at the Roots of Cancer. Cancer Cell 2016, 29 (6), 783-803.
120. Lobikin, M.; Chernet, B.; Lobo, D.; Levin, M., Resting potential, oncogene-induced tumorigenesis, and metastasis: the bioelectric basis of cancer in vivo. Physical biology 2012, 9 (6), 065002.
121. Chernet, B.; Levin, M., Endogenous Voltage Potentials and the Microenvironment: Bioelectric Signals that Reveal, Induce and Normalize Cancer. J Clin Exp Oncol 2013, Suppl 1.
122. Markowska, A.; Kaysiewicz, J.; Markowska, J.; Huczynski, A., Doxycycline, salinomycin, monensin and ivermectin repositioned as cancer drugs. Bioorg Med Chem Lett 2019, 29 (13), 1549-1554.
123. Jiang, L.; Wang, P.; Sun, Y. J.; Wu, Y. J., Ivermectin reverses the drug resistance in cancer cells through EGFR/ERK/Akt/NF-kappaB pathway. J Exp Clin Cancer Res 2019, 38 (1), 265.
124. Intuyod, K.; Hahnvajanawong, C.; Pinlaor, P.; Pinlaor, S., Anti-parasitic Drug Ivermectin Exhibits Potent Anticancer Activity Against Gemcitabine-resistant Cholangiocarcinoma In Vitro. Anticancer Res 2019, 39 (9), 4837-4843.
125. Juarez, M.; Schcolnik-Cabrera, A.; Duenas-Gonzalez, A., The multitargeted drug ivermectin: from an antiparasitic agent to a repositioned cancer drug. Am J Cancer Res 2018, 8 (2), 317-331.
126. Gupta, P. B.; Onder, T. T.; Jiang, G.; Tao, K.; Kuperwasser, C.; Weinberg, R. A.; Lander, E. S., Identification of selective inhibitors of cancer stem cells by high-throughput screening. Cell 2009, 138 (4), 645-59.
127. Fukushiro-Lopes, D. F.; Hegel, A. D.; Rao, V.; Wyatt, D.; Baker, A.; Breuer, E. K.; Osipo, C.; Zartman, J. J.; Burnette, M.; Kaja, S.; Kouzoukas, D.; Burris, S.; Jones, W. K.; Gentile, S., Preclinical study of a Kv11.1 potassium channel activator as antineoplastic approach for breast cancer. Oncotarget 2018, 9 (3), 3321-3337.
128. Gentile, S., hERG1 potassium channel in cancer cells: a tool to reprogram immortality. Eur Biophys J 2016, 45 (7), 649-655.
129. Rao, V. R.; Perez-Neut, M.; Kaja, S.; Gentile, S., Voltage-gated ion channels in cancer cell proliferation. Cancers (Basel) 2015, 7(2), 849-75.
130. Perez-Neut, M.; Shum, A.; Cuevas, B. D.; Miller, R.; Gentile, S., Stimulation of hERG1 channel activity promotes a calcium-dependent degradation of cyclin E2, but not cyclin E1, in breast cancer cells. Oncotarget 2015, 6 (3), 1631-9.
131. Perez-Neut, M.; Rao, V. R.; Gentile, S., hERG1/Kv11.1 activation stimulates transcription of p21waf/cip in breast cancer cells via a calcineurin-dependent mechanism. Oncotarget 2015.
132. Spugnini, E. P.; Citro, G.; Fais, S., Proton pump inhibitors as anti vacuolar-ATPases drugs: a novel anticancer strategy. J Exp Clin Cancer Res 2010, 29, 44.
133. Kale, V. P.; Amin, S. G.; Pandey, M. K., Targeting ion channels for cancer therapy by repurposing the approved drugs. Biochim Biophys Acta 2015, 1848 (10 Pt B), 2747-55.
134. Villalonga, N.; Ferreres, J. C.; Argiles, J. M.; Condom, E.; Felipe, A., Potassium channels are a new target field in anticancer drug design. Recent Pat Anticancer Drug Discov 2007, 2 (3), 212-23.
135. Becchetti, A., Ion channels and transporters in cancer. 1. Ion channels and cell proliferation in cancer. American journal of physiology. Cell physiology 2011, 301 (2), C255-65.
136. Arcangeli, A.; Becchetti, A., New Trends in Cancer Therapy: Targeting Ion Channels and Transporters. Pharmaceuticals 2010, 3 (4), 1202.
137. Arcangeli, A.; Crociani, O.; Lastraioli, E.; Masi, A.; Pillozzi, S.; Becchetti, A., Targeting ion channels in cancer: a novel frontier in antineoplastic therapy. Current medicinal chemistry 2009, 16 (1), 66-93.
138. Churchill, C. D. M.; Winter, P.; Tuszynski, J. A.; Levin, M., EDEn—Electroceutical Design Environment: An Ion Channel Database with Small Molecule Modulators and Tissue Expression Information. iScience 2018, 11, 42-56.
139. Tuszynski, J.; Tilli, T. M.; Levin, M., Ion Channel and Neurotransmitter Modulators as Electroceutical Approaches to the Control of Cancer. Curr Pharm Des 2017, 23 (32), 4827-4841.
140. Pai, V. P.; Cervera, J.; Mafe, S.; Willocq, V.; Lederer, E. K.; Levin, M., HCN2 Channel-Induced Rescue of Brain Teratogenesis via Local and Long-Range Bioelectric Repair. Front Cell Neurosci 2020, 14 (136).
141. Pai, V. P.; Pietak, A.; Willocq, V.; Ye, B.; Shi, N. Q.; Levin, M., HCN2 Rescues brain defects by enforcing endogenous voltage pre-patterns. Nature Communications 2018, 9.
142. Levin, M., Bioelectrical approaches to cancer as a problem of the scaling of the cellular self. Prog Biophys Mol Biol 2021.
143. Tojima, T.; Yamane, Y.; Takahashi, M.; Ito, E., Acquisition of neuronal proteins during differentiation of NG108-15 cells. Neurosci Res 2000, 37 (2), 153-61.
144. Seidman, K. J.; Barsuk, J. H.; Johnson, R. F.; Weyhenmeyer, J. A., Differentiation of NG108-15 neuroblastoma cells by serum starvation or dimethyl sulfoxide results in marked differences in angiotensin II receptor subtype expression. J Neurochem 1996, 66 (3), 1011-8.
145. Ma, W.; Pancrazio, J. J.; Coulombe, M.; Dumm, J.; Sathanoori, R.; Barker, J. L.; Kowtha, V. C.; Stenger, D. A.; Hickman, J. J., Neuronal and glial epitopes and transmitter-synthesizing enzymes appear in parallel with membrane excitability during neuroblastomaxglioma hybrid differentiation. Developmental Brain Research 1998, 106 (1-2), 155-163.
146. Nurse, P., A long twentieth century of the cell cycle and beyond. Cell 2000, 100 (1), 71-8.
147. Rieske, P.; Golanska, E.; Zakrzewska, M.; Piaskowski, S.; Hulas-Bigoszewska, K.; Wolanczyk, M.; Szybka, M.; Witusik-Perkowska, M.; Jaskolski, D. J.; Zakrzewski, K.; Biernat, W.; Krynska, B.; Liberski, P. P., Arrested neural and advanced mesenchymal differentiation of glioblastoma cells-comparative study with neural progenitors. Bmc Cancer 2009, 9, 54.
148. Wolanczyk, M.; Hulas-Bigoszewska, K.; Witusik-Perkowska, M.; Papierz, W.; Jaskolski, D.; Liberski, P. P.; Rieske, P., Imperfect oligodendrocytic and neuronal differentiation of glioblastoma cells. Folia Neuropathol 2010, 48 (1), 27-34.
149. Bilet, A.; Bauer, C. K., Effects of the small molecule HERG activator NS1643 on Kv11.3 channels. PLoS One 2012, 7(11), e50886.
150. Zanconato, F.; Cordenonsi, M.; Piccolo, S., YAP and TAZ: a signalling hub of the tumour microenvironment. Nat Rev Cancer 2019, 19 (8), 454-464.
151. Xie, Q.; Chen, J.; Feng, H.; Peng, S.; Adams, U.; Bai, Y.; Huang, L.; Li, J.; Huang, J.; Meng, S.; Yuan, Z., YAP/TEAD-mediated transcription controls cellular senescence. Cancer Res 2013, 73 (12), 3615-24.
152. Wolff, C.; Fuks, B.; Chatelain, P., Comparative study of membrane potential-sensitive fluorescent probes and their use in ion channel screening assays. J Biomol Screen 2003, 8 (5), 533-43.
153. Yamada, A.; Gaja, N.; Ohya, S.; Muraki, K.; Narita, H.; Ohwada, T.; Imaizumi, Y., Usefulness and limitation of DiBAC4(3), a voltage-sensitive fluorescent dye, for the measurement of membrane potentials regulated by recombinant large conductance Ca2+-activated K+ channels in HEK293 cells. Jpn J Pharmacol 2001, 86 (3), 342-50.
154. Perez-Neut, M.; Haar, L.; Rao, V.; Santha, S.; Lansu, K.; Rana, B.; Jones, W. K.; Gentile, S., Activation of hERG3 channel stimulates autophagy and promotes cellular senescence in melanoma. Oncotarget 2016, 7 (16), 21991-2004.
155. Santini, M. P.; Talora, C.; Seki, T.; Bolgan, L.; Dotto, G. P., Cross talk among calcineurin, Sp1/Sp3, and NFAT in control of p21(WAF1/CIP1) expression in keratinocyte differentiation. Proc Natl Acad Sci USA 2001, 98(17), 9575-80.
156. Moon, J. H.; Hong, J. M.; Park, S. Y., Calcineurin Activation by Prion Protein Induces Neurotoxicity via Mitochondrial Reactive Oxygen Species. Oxid Med Cell Longev 2021, 2021, 5572129.
157. Livnat-Levanon, N.; Kevei, E.; Kleifeld, O.; Krutauz, D.; Segref, A.; Rinaldi, T.; Erpapazoglou, Z.; Cohen, M.; Reis, N.; Hoppe, T.; Glickman, M. H., Reversible 26S proteasome disassembly upon mitochondrial stress. Cell Rep 2014, 7(5), 1371-1380.
158. Kudo, Y.; Takata, T.; Ogawa, I.; Kaneda, T.; Sato, S.; Takekoshi, T.; Zhao, M.; Miyauchi, M.; Nikai, H., p27Kip1 accumulation by inhibition of proteasome function induces apoptosis in oral squamous cell carcinoma cells. Clin Cancer Res 2000, 6 (3), 916-23.
159. Kossatz, U.; Dietrich, N.; Zender, L.; Buer, J.; Manns, M. P.; Malek, N. P., Skp2-dependent degradation of p27kip1 is essential for cell cycle progression. Gene Dev 2004, 18 (21), 2602-2607.
160. Lee, J. J.; Kim, B. C.; Park, M. J.; Lee, Y. S.; Kim, Y. N.; Lee, B. L.; Lee, J. S., PTEN status switches cell fate between premature senescence and apoptosis in glioma exposed to ionizing radiation. Cell Death Differ 2011, 18 (4), 666-77.
161. Tompson, D. J.; Crean, C. S.; Reeve, R.; Berry, N. S., Efficacy and tolerability exposure-response relationship of retigabine (ezogabine) immediate-release tablets in patients with partial-onset seizures. Clin Ther 2013, 35 (8), 1174-1185 e4.
162. Large, C. H.; Sokal, D. M.; Nehlig, A.; Gunthorpe, M. J.; Sankar, R.; Crean, C. S.; Vanlandingham, K. E.; White, H. S., The spectrum of anticonvulsant efficacy of retigabine (ezogabine) in animal models: implications for clinical use. Epilepsia 2012, 53 (3), 425-36.
163. Myatt, D. P., The correlation of plasma proteins binding capacity and flavopiridol cellular and clinical trial studies. Biomedical Spectroscopy and Imaging 2017, 6 (1-2), 59-73.
164. Basa-Denes, O.; Angi, R.; Karpati, B.; Jordan, T.; Otvos, Z.; Erdosi, N.; Ujhelyi, A.; Ordasi, B.; Molnar, L.; McDermott, J.; Roe, C.; McKenzie, L.; Solymosi, T.; Heltovics, G.; Glavinas, H., Dose Escalation Study to Assess the Pharmacokinetic Parameters of a Nano-amorphous Oral Sirolimus Formulation in Healthy Volunteers. Eur J Drug Metab Pharmacokinet 2019, 44 (6), 777-785.
165. Cloughesy, T. F.; Yoshimoto, K.; Nghiemphu, P.; Brown, K.; Dang, J.; Zhu, S.; Hsueh, T.; Chen, Y.; Wang, W.; Youngkin, D.; Liau, L.; Martin, N.; Becker, D.; Bergsneider, M.; Lai, A.; Green, R.; Oglesby, T.; Koleto, M.; Trent, J.; Horvath, S.; Mischel, P. S.; Mellinghoff, I. K.; Sawyers, C. L., Antitumor activity of rapamycin in a Phase I trial for patients with recurrent PTEN-deficient glioblastoma. PLoS Med 2008, 5 (1), e8.
166. Cheer, S. M.; Prakash, A.; Faulds, D.; Lamb, H. M., Pantoprazole: an update of its pharmacological properties and therapeutic use in the management of acid-related disorders. Drugs 2003, 63 (1), 101-33.
167. Sigaroudi, A.; Stelzer, C.; Braun, T.; Frechen, S.; Huttner, S.; Schroter, M.; Kinzig, M.; Fuhr, U.; Holzgrabe, U.; Sorgel, F., Comparison of Pantoprazole Concentrations in Simultaneous Cerebrospinal Fluid and Serum Samples. Pharmacology 2016, 98 (1-2), 70-2.
168. Terstappen, G. C.; Meyer, A. H.; Bell, R. D.; Zhang, W., Strategies for delivering therapeutics across the blood-brain barrier. Nat Rev Drug Discov 2021, 20 (5), 362-383.
169. Lugini, L.; Federici, C.; Borghi, M.; Azzarito, T.; Marino, M. L.; Cesolini, A.; Spugnini, E. P.; Fais, S., Proton pump inhibitors while belonging to the same family of generic drugs show different anti-tumor effect. J Enzyme Inhib Med Chem 2016, 31 (4), 538-45.
170. Urrego, D.; Tomczak, A. P.; Zahed, F.; Stuhmer, W.; Pardo, L. A., Potassium channels in cell cycle and cell proliferation. Philos Trans R Soc Lond B Biol Sci 2014, 369 (1638), 20130094.
171. Ghiani, C. A.; Yuan, X.; Eisen, A. M.; Knutson, P. L.; DePinho, R. A.; McBain, C. J.; Gallo, V., Voltage-Activated K+ Channels and Membrane Depolarization Regulate Accumulation of the Cyclin-Dependent Kinase Inhibitors p27Kip1 and p21CIP1 in Glial Progenitor Cells. The Journal of Neuroscience 1999, 19 (13), 5380-5392.
172. Chittajallu, R.; Chen, Y.; Wang, H.; Yuan, X.; Ghiani, C. A.; Heckman, T.; McBain, C. J.; Gallo, V., Regulation of Kv1 subunit expression in oligodendrocyte progenitor cells and their role in G1/S phase progression of the cell cycle. Proc Natl Acad Sci USA 2002, 99 (4), 2350-5.
173. Harley, W.; Floyd, C.; Dunn, T.; Zhang, X. D.; Chen, T. Y.; Hegde, M.; Palandoken, H.; Nantz, M. H.; Leon, L.; Carraway, K. L., 3rd; Lyeth, B.; Gorin, F. A., Dual inhibition of sodium-mediated proton and calcium efflux triggers non-apoptotic cell death in malignant gliomas. Brain Res 2010, 1363, 159-69.
174. Marathe, K.; McVicar, N.; Li, A.; Bellyou, M.; Meakin, S.; Bartha, R., Topiramate induces acute intracellular acidification in glioblastoma. J Neurooncol 2016, 130 (3), 465-472.
175. Gatti, M.; Pinato, S.; Maspero, E.; Soffientini, P.; Polo, S.; Penengo, L., A novel ubiquitin mark at the N-terminal tail of histone H2As targeted by RNF168 ubiquitin ligase. Cell Cycle 2012, 11 (13), 2538-44.
176. Wu, L.; Bernal, G. M.; Cahill, K. E.; Pytel, P.; Fitzpatrick, C. A.; Mashek, H.; Weichselbaum, R. R.; Yamini, B., BCL3 expression promotes resistance to alkylating chemotherapy in gliomas. Sci Transl Med 2018, 10 (448).
177. Liu, Y.; Tang, Z. G.; Yang, J. Q.; Zhou, Y.; Meng, L. H.; Wang, H.; Li, C. L., Low concentration of quercetin antagonizes the invasion and angiogenesis of human glioblastoma U251 cells. Onco Targets Ther 2017, 10, 4023-4028.
178. Vengoji, R.; Macha, M. A.; Batra, S. K.; Shonka, N. A., Natural products: a hope for glioblastoma patients. Oncotarget 2018, 9 (31), 22194-22219.
179. Cenciarini, M.; Valentino, M.; Belia, S.; Sforna, L.; Rosa, P.; Ronchetti, S.; D'Adamo, M. C.; Pessia, M., Dexamethasone in Glioblastoma Multiforme Therapy: Mechanisms and Controversies. Front Mol Neurosci 2019, 12, 65.
180. Bernstock, J. D.; Ye, D.; Gessler, F. A.; Lee, Y. J.; Peruzzotti-Jametti, L.; Baumgarten, P.; Johnson, K. R.; Maric, D.; Yang, W.; Kogel, D.; Pluchino, S.; Hallenbeck, J. M., Topotecan is a potent inhibitor of SUMOylation in glioblastoma multiforme and alters both cellular replication and metabolic programming. Sci Rep 2017, 7(1), 7425.
181. Morgan, R. J.; Synold, T.; Mamelak, A.; Lim, D.; Al-Kadhimi, Z.; Twardowski, P.; Leong, L.; Chow, W.; Margolin, K.; Shibata, S.; Somlo, G.; Yen, Y.; Frankel, P.; Doroshow, J. H., Plasma and cerebrospinal fluid pharmacokinetics of topotecan in a phase I trial of topotecan, tamoxifen, and carboplatin, in the treatment of recurrent or refractory brain or spinal cord tumors. Cancer Chemother Pharmacol 2010, 66 (5), 927-33.
182. Macdonald, D.; Cairncross, G.; Stewart, D.; Forsyth, P.; Sawka, C.; Wainman, N.; Eisenhauer, E., Phase II study of topotecan in patients with recurrent malignant glioma. National Clinical Institute of Canada Clinical Trials Group. Ann Oncol 1996, 7 (2), 205-7.
183. Cho, CKD-602, a camptothecin derivative, inhibits proliferation and induces apoptosis in glioma cell lines. Oncology Reports 2009, 21 (6).
184. Graham, C. D.; Kaza, N.; Klocke, B. J.; Gillespie, G. Y.; Shevde, L. A.; Carroll, S. L.; Roth, K. A., Tamoxifen Induces Cytotoxic Autophagy in Glioblastoma. J Neuropathol Exp Neurol 2016, 75 (10), 946-954.
185. Wan, W.; Zhang, X.; Huang, C.; Chen, L.; Yang, X.; Bao, K.; Peng, T., Monensin inhibits glioblastoma angiogenesis via targeting multiple growth factor receptor signaling. Biochem Biophys Res Commun 2020, 530 (2), 479-484.
186. Yoon, M. J.; Kang, Y. J.; Kim, I. Y.; Kim, E. H.; Lee, J. A.; Lim, J. H.; Kwon, T. K.; Choi, K. S., Monensin, a polyether ionophore antibiotic, overcomes TRAIL resistance in glioma cells via endoplasmic reticulum stress, DR5 upregulation and c-FLIP downregulation. Carcinogenesis 2013, 34 (8), 1918-28.
187. Enriquez Perez, J.; Fritzell, S.; Kopecky, J.; Visse, E.; Darabi, A.; Siesjo, P., The effect of locally delivered cisplatin is dependent on an intact immune function in an experimental glioma model. Sci Rep 2019, 9 (1), 5632.
188. Nakagawa, H.; Sasagawa, S.; Itoh, K., Sodium butyrate induces senescence and inhibits the invasiveness of glioblastoma cells. Oncol Lett 2018, 15 (2), 1495-1502.
189. Engelhard, H. H.; Duncan, H. A.; Kim, S.; Criswell, P. S.; Van Eldik, L., Therapeutic effects of sodium butyrate on glioma cells in vitro and in the rat C6 glioma model. Neurosurgery 2001, 48 (3), 616-24; discussion 624-5.

### Example 3—Ion Channel Drugs Arrest Cell Cycles of NG108-15 and U87 Cells

The most promising compounds from the results of experiments using mouse/rat neuroblastoma/glioma NG108-15 cells containing the FUCCI cell cycle reporter and palmitoyl-mTurquoise2 fluorescent membrane tag (e.g. showing greatest reductions in cancer cell proliferation) were tested again to obtain at least three biological replicates. Those compound combinations that showed significance activity on NG108-15 cells were then screened against the human glioblastoma U87 cell line containing the FUCCI cell cycle reporter and palmitoyl-mTurquoise2 fluorescent membrane tag. Follow up biological replicates were done on the positive hits (showing reduced glioblastoma cell proliferation) from the initial U87 cell screen. Antibodies for a variety of cell markers for differentiation, proliferation, and apoptosis were used to stain both NG108-15 and U87 cells that were treated with the most promising drug combinations for 6 days and then fixed. Senescence assays and BrdU incorporation assays were done on cells treated the same way. To confirm the mechanism of action of these treatments (control of bioelectric state in the cells), automated patch clamping data were obtained on NG108-15 cells treated with some of the most promising drug combinations immediately after application to investigate effects on resting membrane potential. Functional maturation determination was also performed by examining calcium signaling for some of the best performing compounds and combinations. Finally, a live/dead assay was performed on human induced neural stem cells that were differentiated for 7 days prior to a three-day treatment with the most promising compound combinations in both NG108-15 and U87 cells.

Strategy: the compounds chosen for testing included compounds known to alter the membrane potential of the cell by creating either an acidic environment inside the cell and an alkaline environment outside the cell, increasing potassium efflux, or decreasing sodium influx. Rapamycin was chosen for its combinatorial effects with the ion modulating drugs due to its ability to induce autophagy. The most effective combinations were able to arrest the cell cycle long enough to enable terminal differentiation, senescence, or apoptosis. All tested compounds and combinations were compared to temozolomide treatments, the standard clinical therapy for glioblastoma. The list of the most effective compounds tested, and their function are listed in Table 2, above.

In NG108-15 cells, FIG. 1 shows the best of the individual and combination treatments when compared to control at day 6. On these bar graphs, higher (e.g., control bars) represent more cells, and lower represents reduction in cancer cell proliferation (the effect we're looking for). The stacked bar graphs show the ratio of cells in different parts of the cell cycle. Increased red (G1) and orange (early S) fractions indicate cell cycle arrest. A reduction of the yellow fraction (actively dividing) and an increase in either the red or orange (in transition to start dividing) fraction is desired.

FIG. 1-A shows that in NG108-15 cells treated with NS1643 (hERG activator) at 20 μM and 50 μM alone were able to lower proliferation significantly. These two concentrations of NS1643 were very effective at lowing cell proliferation when combined with pantoprazole (a proton pump inhibitor) and worked better than pantoprazole or NS1643 alone. NS1643 at 50 μM also significantly lowered proliferation when combined with rapamycin (autophagy inducer) and worked better than rapamycin or NS1643 alone. Retigabine (voltage activated potassium channel opener) by itself significantly lowered cell proliferation, but the combination of retigabine with rapamycin or pantoprazole worked better than any of the compounds alone. Pantoprazole at 100 μM was the most effective compound alone or in combination with lamotrigine (blocks voltage gated sodium channels), NS1643, and rapamycin. These three combinations worked better than one of the positive controls, cAMP with rapamycin at 200 nM in full serum media, which is known to terminally differentiate these cells when in low serum (the other positive control). However, cAMP cannot be used clinically due to its many off-target effects.

The cell cycle data in FIG. 1-B reveal that pantoprazole increases the proportion of cells in early S and that its combinations can also increase the proportion of cells in G1. Rapamycin treatment increased the proportion of cells in G1, while NS1643 treatment did not seem to affect the cell cycle proportion. Currently, no publications have shown the efficacy of NS1643 on glioblastoma or its efficacy in combination with rapamycin or pantoprazole. However, this drug has been published to be effective towards breast cancer and melanoma. Pantoprazole has been shown to reduce glioblastoma proliferation in vitro but no studies have shown the efficacy of pantoprazole treatment in combination with NS1643, lamotrigine, retigabine, or rapamycin. It is worthwhile to note that temozolomide (standard glioblastoma treatment) was not effective on NG108-15 cells.

FIG. 2 shows the pantoprazole combinations that worked significantly better than pantoprazole alone in NG108-15 cells. FIG. 2-A shows that the combination of pantoprazole with NS1643 at 20 μM, lamotrigine at 100 μM and rapamycin at 100 nM were the only combinations that showed significantly more efficacy than pantoprazole alone at reducing cell proliferation after 6 days of treatment, with the combination with rapamycin showing the most significant difference. FIG. 2-B shows that that the proportion of cells in G1 and early S only slightly increased for pantoprazole treatments in combination with NS1643 and lamotrigine, but that the combination with rapamycin did increase the G1 proportion by quite a bit.

To understand whether these drug treatments would terminally differentiate and thus stop the proliferation of the cells after the drugs were withdrawn, a recovery test was performed (FIG. 3). Cells were treated with the drugs for 6 days, then the drugs were removed (demarcated by the dashed line in FIG. 3), and the cells were put into media containing no drugs for another 10 days.

FIG. 3 shows that although the three combinations with pantoprazole showed significantly less cell proliferation after 6 days of treatment, they still showed some recovery after treatment was removed, but that it was less than pantoprazole alone, but not significantly so. However, the positive control showed no recovery and was more indicative of cells that had terminally differentiated or were dying after treatment.

FIG. 4 shows the results of U87 human glioblastoma cells analyzed the same way as NG108-15 cells above. FIG. 4-A shows that in human glioblastoma U87 cells the same drug treatments that were significantly effective in NG108-15 cells were also significantly effective compared to control after 6 days. Notably, NS1643 at 50 μM significantly decreased cell proliferation as compared to control, but was much more effective when combined with pantoprazole, TMZ, and rapamycin. Pantoprazole treatment also worked very well in this cell line and showed very significant differences in cell proliferation as compared to control when combined with rapamycin, retigabine, NS1643, lamotrigine, or TMZ. The data showed that in U87 cells, TMZ was very effective at reducing cell number as compared to control, but combinations with minoxidil, lamotrigine, pantoprazole, rapamycin, retigabine, and NS1643 increased the effectiveness of TMZ. The cell cycle data in FIG. 4-B shows that some of the most effective combinations increased the G1 and early S proportion of cells but not all. Pantoprazole showed its characteristic increase in the early S proportion of cells and rapamycin an increase in the G1 proportion seen in the NG108-15 cells. TMZ and NS1643 treatment did not show much change in proportion of cells in each stage of the cell cycle as compared to control. Unfortunately, the proportion of cells in each cell cycle stage for the best treatment in U87 cells, pantoprazole with retigabine, was not observed due to autofluorescent aggregation in the cytoplasm of the cells that caused spurious results in the FUCCI reporter data.

FIG. 5-A showed that pantoprazole alone was significantly more effective at reducing cell proliferation after 6 days of treatment than TMZ alone. All combinations that were more effective than TMZ alone were those that were with TMZ or with pantoprazole. Rapamycin, NS1643, and pantoprazole all increased the effectiveness of TMZ significantly, with NS1643 and pantoprazole being the most significant of these combinations with TMZ. FIG. 5-B showed that TMZ in combination with rapamycin increased the proportion of cells in G1 and that combinations with NS1643 increased the proportion of cells in early S as compared to TMZ alone. Pantoprazole combinations consistently showed a larger proportion of cells in early S as compared to TMZ alone with a complimentary decrease of cells in late S, G2, and M.

FIG. 6 takes a closer look at the significance between the different pantoprazole concentration and pantoprazole alone in U87 cells. FIG. 6-A shows that, in U87 cells, the same combinations that were significantly better than pantoprazole in NG108-15 cells were also significant along with the added combinations of TMZ or retigabine. Pantoprazole in combination with rapamycin, lamotrigine, NS1643, TMZ or retigabine were all significantly better than pantoprazole alone. The most significant combinations were with NS1643 and retigabine. The combination of pantoprazole with NS1643 was so effective that cutting the pantoprazole concentration by half and combining it with NS1643 at 50 μM was significantly more effective than pantoprazole at 100 μM alone. FIG. 6-B shows the characteristic increase in G1 when pantoprazole was combined with rapamycin and that the combination with NS1643 increased the proportion of the cells in early S, with a complimentary decrease in late S, G2, and M.

A recovery test was also done on the U87 cells, FIG. 7 looks only at those treatments that were significantly better than pantoprazole at lowering cell proliferation at day 6. FIG. 7 shows that the combinations of pantoprazole with rapamycin show similar recovery after day 6 as pantoprazole alone. However, combinations of pantoprazole with retigabine or NS1643 at 50 μM show much more reduced recovery than pantoprazole alone. Suggesting that treatment is permanently changing the cells so that proliferation capability cannot be recovered. Surprisingly the positive control using cAMP in combination with rapamycin started proliferating after day 8.

To investigate mechanistic features of these treatments, automated patch clamp data was collected looking at the electrophysiology of NG108-15 cells that were treated with some of the better performing drug combinations in the cell proliferation assays (FIG. 8). Untreated cells were patched, baseline measurements were taken and then the drugs were added and the changes in their resting membrane potential were recorded.

FIG. 8 shows that rapamycin, retigabine, NS1643, TMZ, and lamotrigine all significantly hyperpolarized the cells as compared to the control. What was surprising was that while pantoprazole did not have an immediate effect on the membrane potential of the cells, its combination with retigabine and rapamycin depolarized the cells instead of hyperpolarized them. The combination of pantoprazole with lamotrigine and NS1643 did not significantly change the membrane potential as compared to control. However, this electrophysiological analysis shows what immediate changes the compounds have on membrane potential and do not show what the changes in potential are over time. Pantoprazole is known to have an inhibitory effect on the transcription of V-ATPases in cancer cells and could result in changes to membrane potential over longer periods of time. The sustained depolarization of cells seen in the combination of pantoprazole with retigabine and rapamycin could have effects on the ability of the cell cycle to proceed if the needed level of hyperpolarization to proceed through S phase is not reached. The FUCCI data showed that pantoprazole and its combinations did increase the proportion of cells in early S and the proliferation data showed that the cells do slow their proliferation significantly.

In addition to this electrophysiology data, calcium event analysis was performed on some of the most promising combinations of drugs on NG108-15 cells, but no after multiple tests no significant findings were observed. This may indicate that rather than terminally differentiating the cells, the treatments are stalling the cell cycle long enough that the cells can no longer re-enter. This may result in cells that are senescent or show some other form of cell cycle exit. To test this, antibody staining of the NG108-15 cells and U87 cells was performed with some of the best treatments.

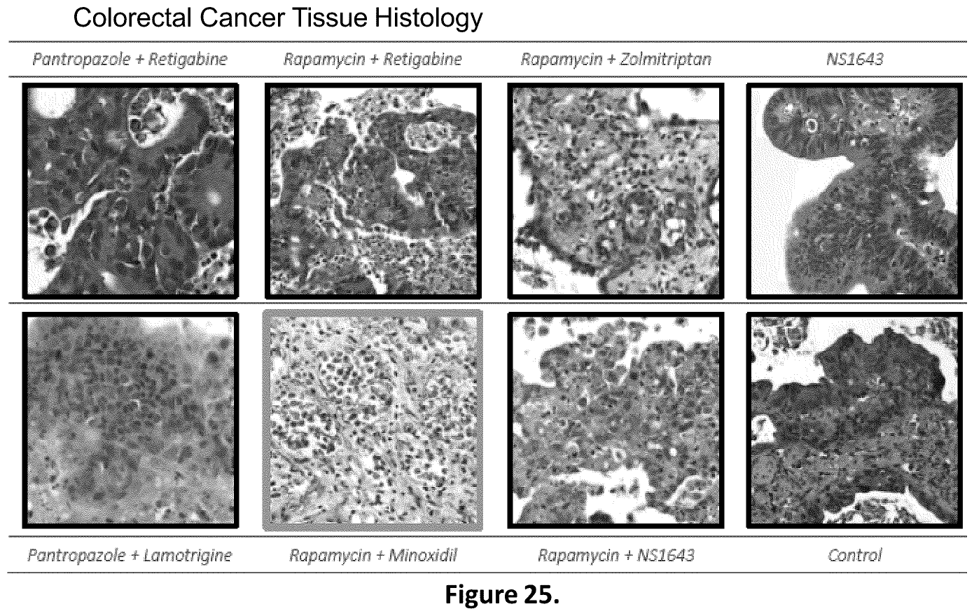

In addition to these assays, the neuronal toxicity of the best performing compounds was investigated. Human induced pluripotent stem cells derived from fibroblasts and made to commit to a neuronal stem cell lineage, called hiNSCs were differentiated for 7 days in neuronal media and then treated with drug for 3 days. The cells used for this assay did not adhere well to plates and showed some cell death even in the control, so the treatment was limited to 3 days in order to be able to perform the Live/Dead assay without too much cell detachment. FIG. 25 shows the percent of cells that died after treatment and compares to control.

Toxicity analysis in hiNSCs differentiated to neurons for 7 days then treated with drugs for 3 days shows that only three treatments showed some toxicity. Pantoprazole showed a slight increase in toxicity that was significant compared to control, and pantoprazole with lamotrigine also showed a slight increase but was more significant than pantoprazole alone when compared to control. NS1643 at 50 μM in combination with TMZ showed the most toxicity when compared to control. However, the difference between the control toxicity and the most toxic combination of NS1643 at 50 μM and TMZ was still only 5.7% higher than control. Considering the high death percentage of the control cells and the delicate nature of these cells, the toxicity appears to be negligible. The rest of the treatments showed no significance compared to control.

In summary, initial screening of a large number of drug treatments on NG108-15 cells with a cell cycle reporter successfully provided many positive hits that were also effective in inhibiting proliferation of human glioblastoma U87 cells. At least 13 drugs and/or drug combinations were found to be significantly more effective than the gold standard in current glioblastoma treatment. These drug combination treatments showed slowed proliferation even after drug removal after 6 days of treatment. The electrophysiology and calcium event data suggest that some of these drug combinations may not be working by hyperpolarization followed by terminal differentiation but rather by a cell cycle disruption that causes the cells to lose the ability to proliferate. The neuronal toxicity data showed that the most promising drug treatments at some of their highest concentrations did not show a very high level of undesirable toxicity as compared to control. Only three treatments showed undesirable toxicity as compared to control with the most toxic only increasing toxicity by 5.7%.

### Example 4—Tumor Tissue Effects of Ion Channel Drugs and Drug Combinations

A tumor tissue cytotoxicity assay was used to evaluate the sensitivities of different tumor tissues to different drugs and drug combinations. This tumor tissue cytotoxicity assay uses a tissue platform which preserves the cancer tissue architecture, including vasculature and immune tissues, thereby more accurately reflecting cancer growth in the body (see e.g. Ben-Hamo, R. et al., Predicting and affecting response to cancer therapy based on pathway-level biomarkers. Nature Communications 11(1), 3296 (2020)). This tumor tissue cytotoxicity assay is capable of providing a quantitative read out of the functional effects of different drugs on specific tumors in the context of the tumor tissue's unique microenvironment. The tumor microenvironment can influence the efficacy of drugs, for example, via hypoxia responses, extracellular matrix changes, and immune responses. For example, the tumor microenvironment is capable of conferring drug resistance in vivo to tumor cells that otherwise are drug sensitive in vitro. In addition, this tumor tissue cytotoxicity assay allows for evaluating anti-tumor drugs whose mechanism of action occurs via microenvironment and/or tissue features, such as vasculature or immune tissues. Further, this assay can maintain tumor tissue structural morphology even after 10 days in culture, which allows for evaluating drugs which require many days to achieve cytotoxicity.

Different drugs and drug combinations (7) were evaluated for cytotoxic effects on breast cancer and colorectal cancer tissues using the tumor tissue cytotoxicity assay as described below. The experiment included an analysis of the cytotoxicity of 7 drug treatments on colorectal cancer tumors (N=3) and breast cancer tumors (N=3). Prior to the initiation of each drug combination treatment, calibration experiments (N=2) were performed on each type of tissue with the individual drug at three different concentrations to determine the most appropriate concentrations. Tumor sections were cultured for at least five days in the presence of a drug, or a combination of drugs. At a selected time-point (e.g. day 5 or day 10), tumor sections were fixed and processed to produce stained sections with morphological and histological markers. The drugs tested were pantoprazole, retigabine, lamotrigine, rapamycin, minoxidil, zolmitriptan, and NS1643, either alone or in combination as shown in Table 3.

TABLE 3
Drug Treatments Tested in the Tumor Tissue Cytotoxicity Assay

1. Pantoprazole 100 μM and Retigabine 10 μM for 6 days
2. Pantoprazole 100 μM and Lamotrigine 100 μM for 6 days
3. Rapamycin 200 nM and Retigabine 10 μM for 6 days
4. Rapamycin 200 nM and Minoxidil 30 μM for 6 days
5. Rapamycin 200 nM and Zolmitriptan 10 μM for 6 days
6. Rapamycin 200 nM and NS1643 50 μM for 6 days
7. NS1643 50 μM alone for 6 days

The viability and morphology of cancer cells in the treated tissues were analyzed and scored on a scale of 0-100 as assessed by various parameters, including by nuclear details, tissue cohesiveness, cytoplasmic changes, and immunohistochemistry staining. On this scale, a higher score correlates with improved clinical responses and scores above 50 have a higher chance to yield a clinical effect. Scores lower than 30 are considered as no response.

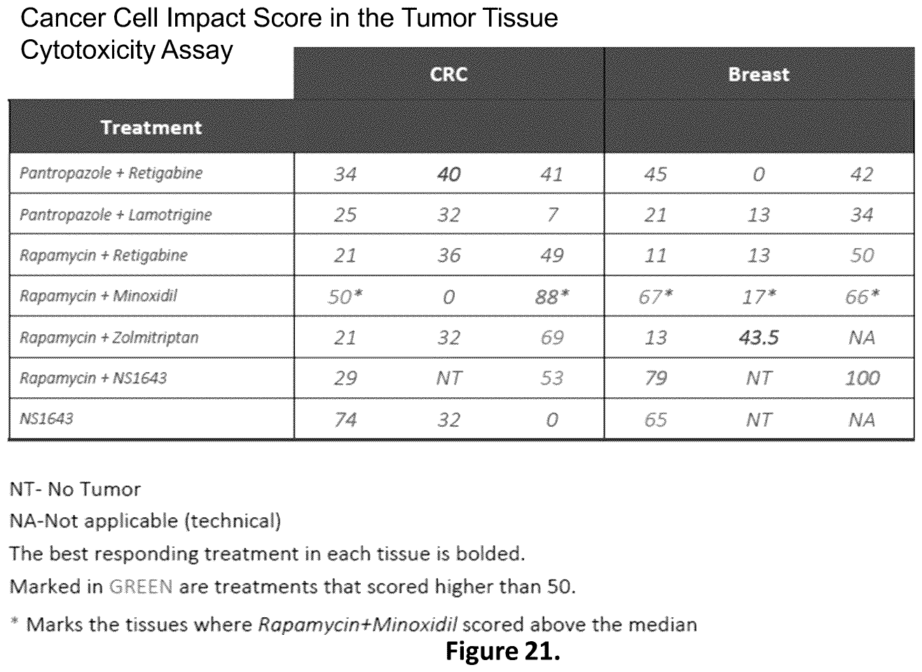

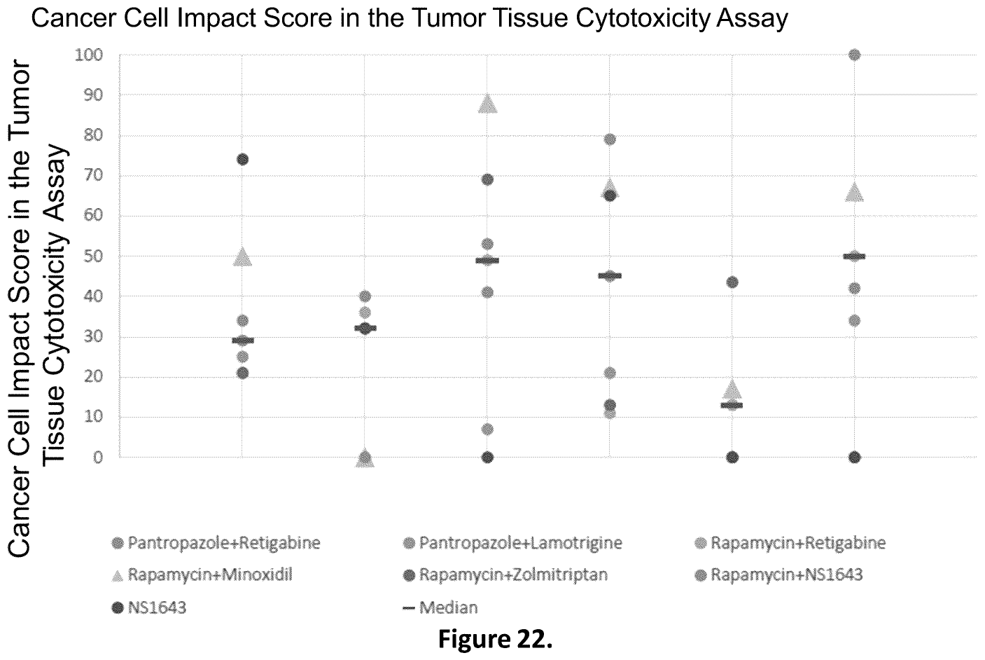

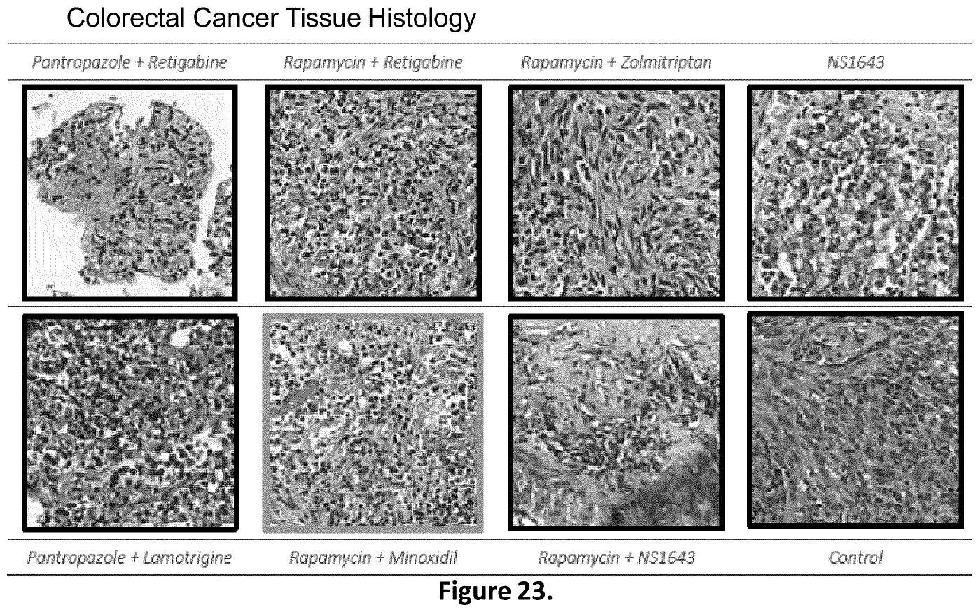

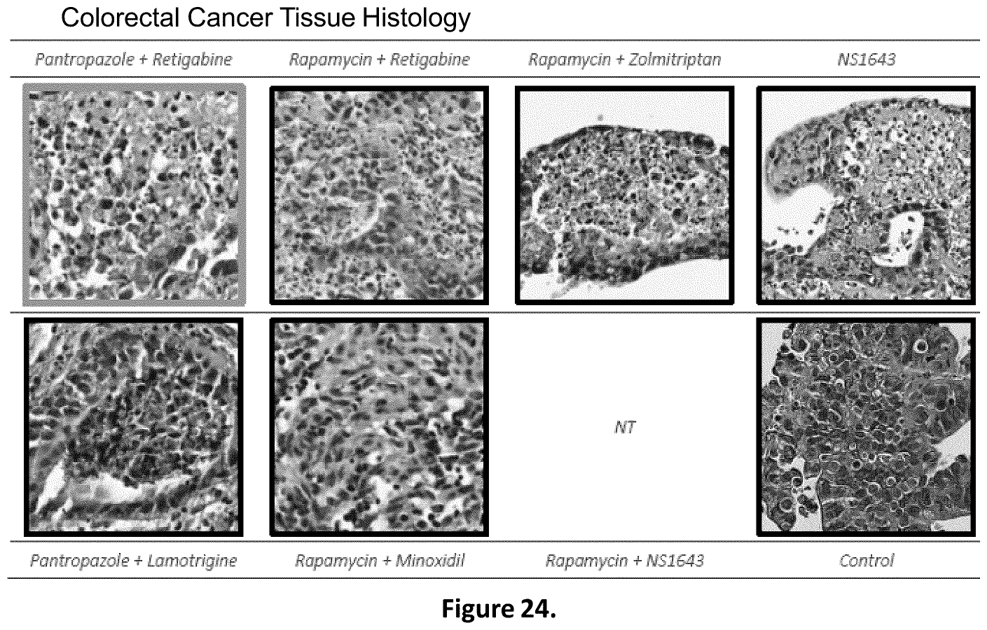

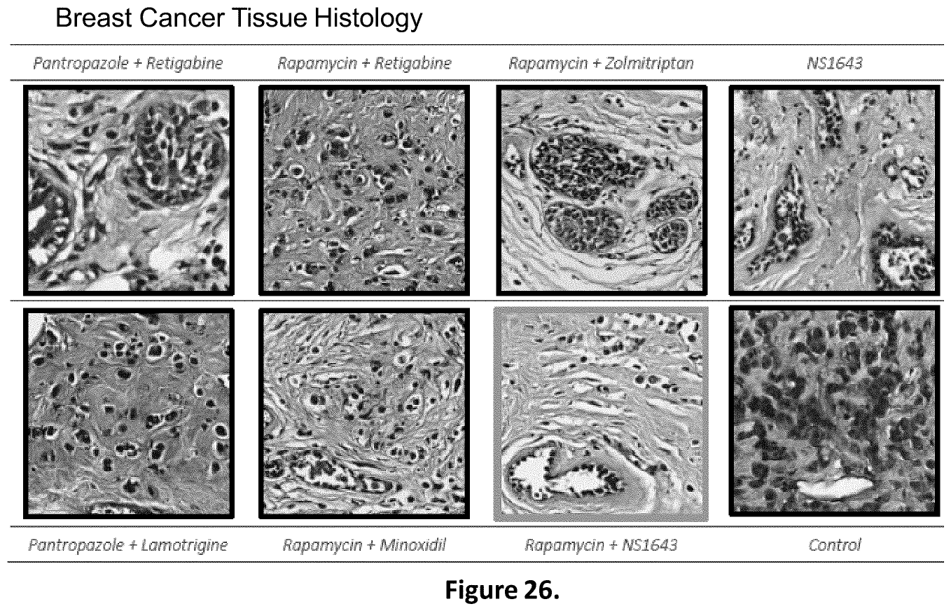

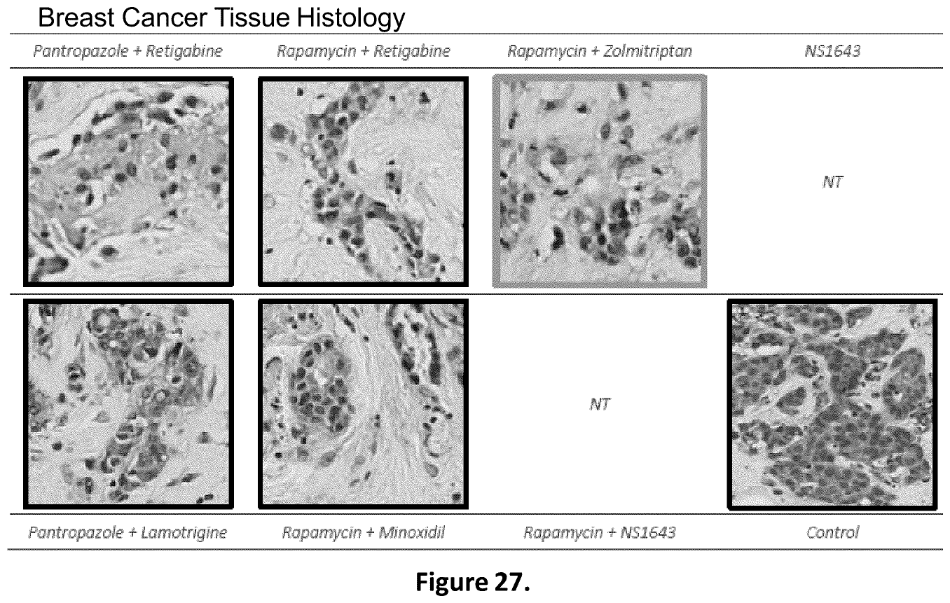

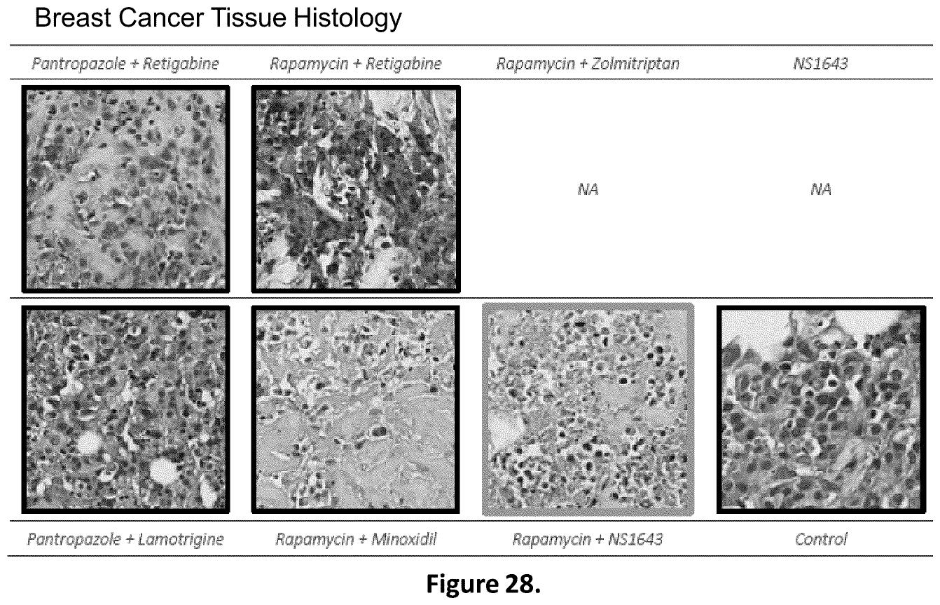

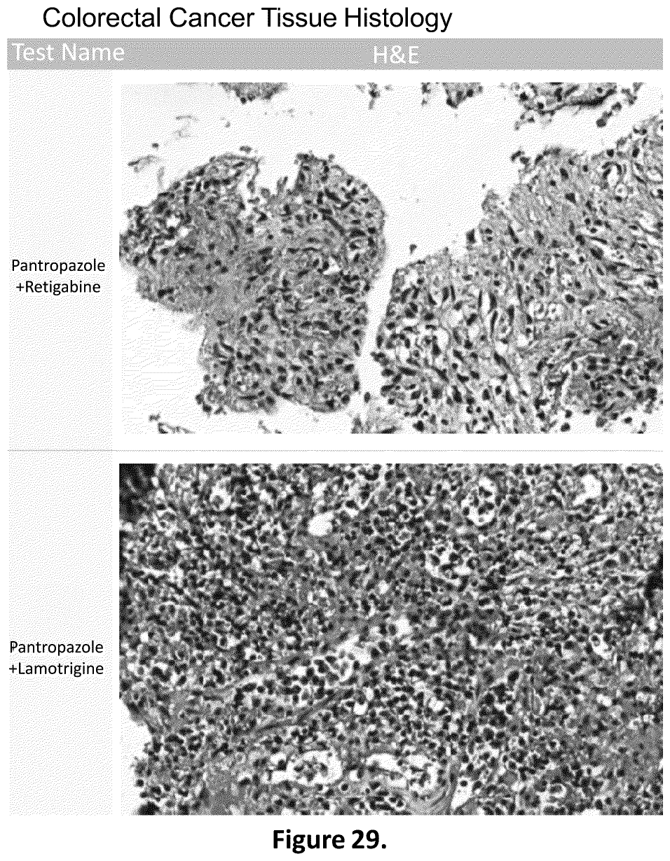

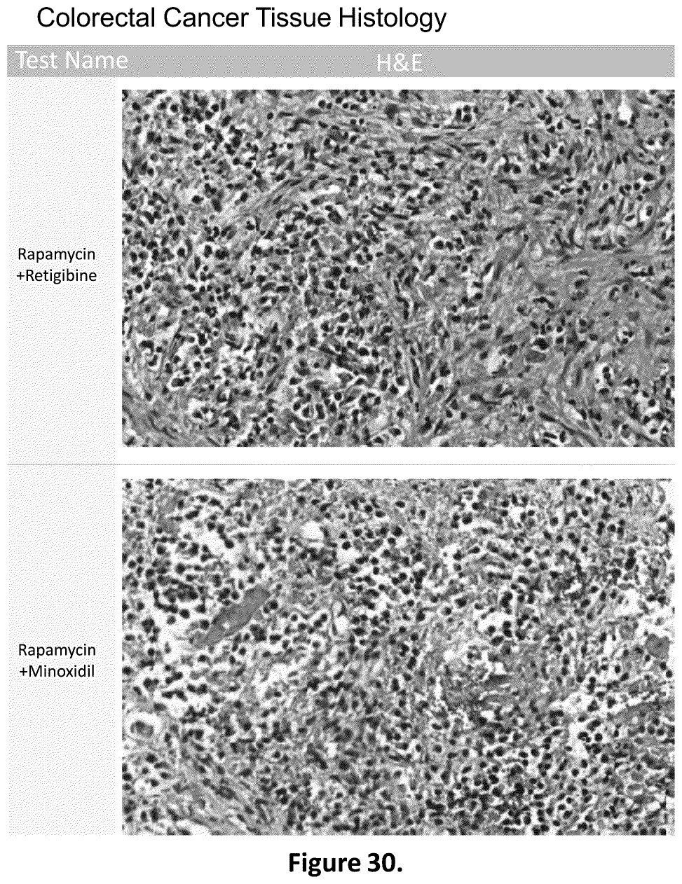

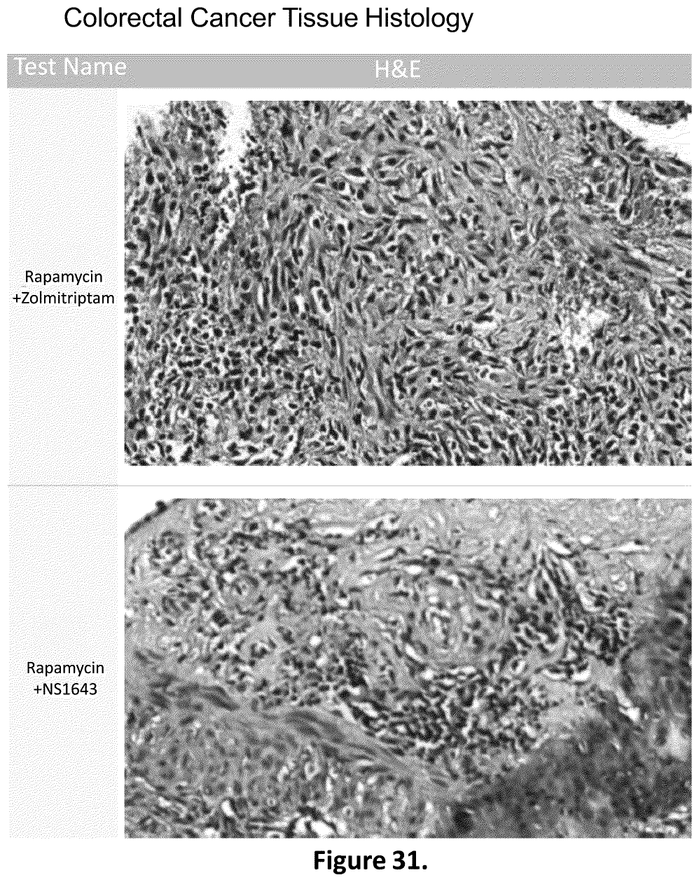

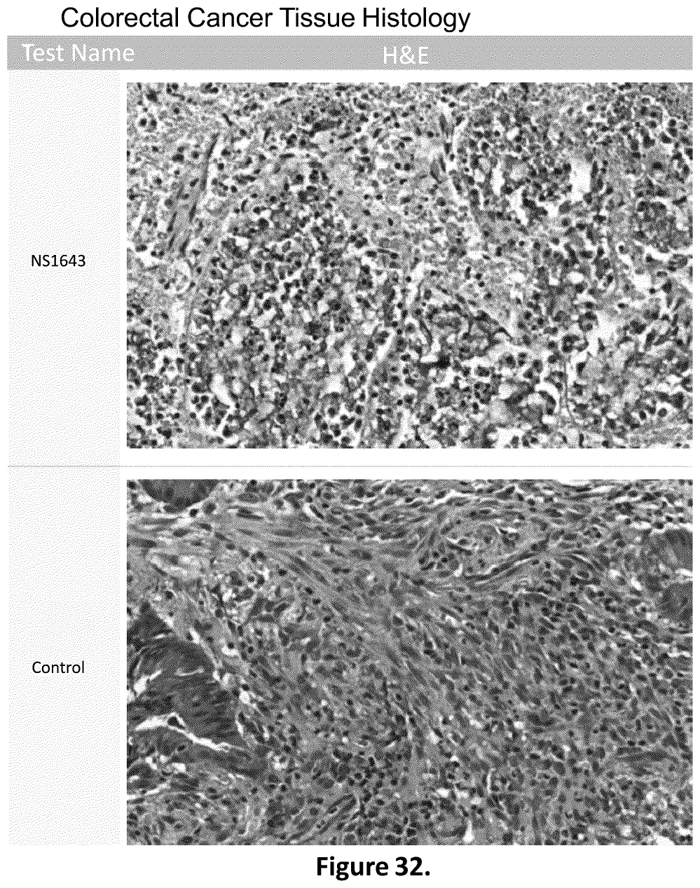

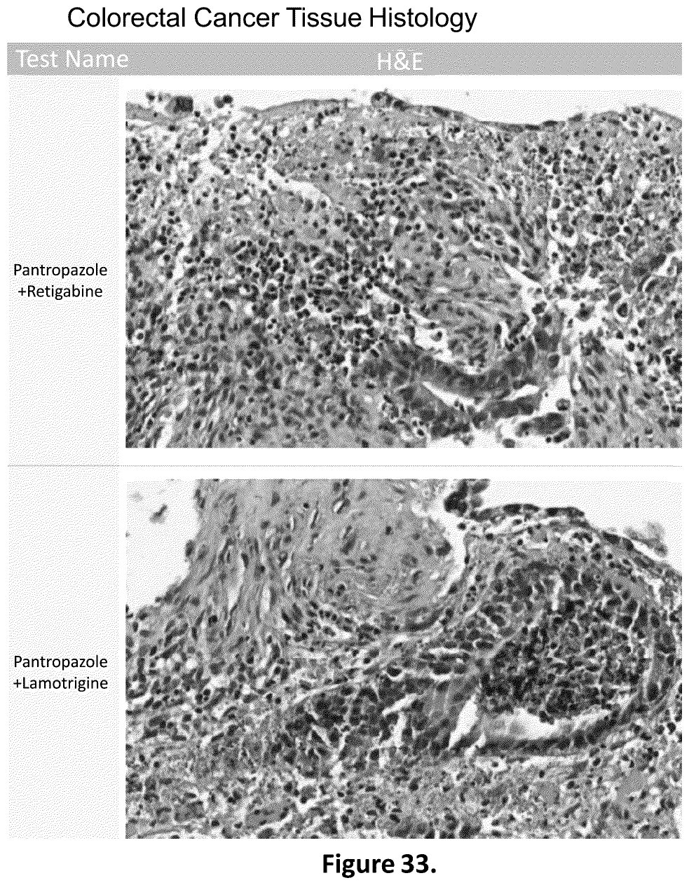

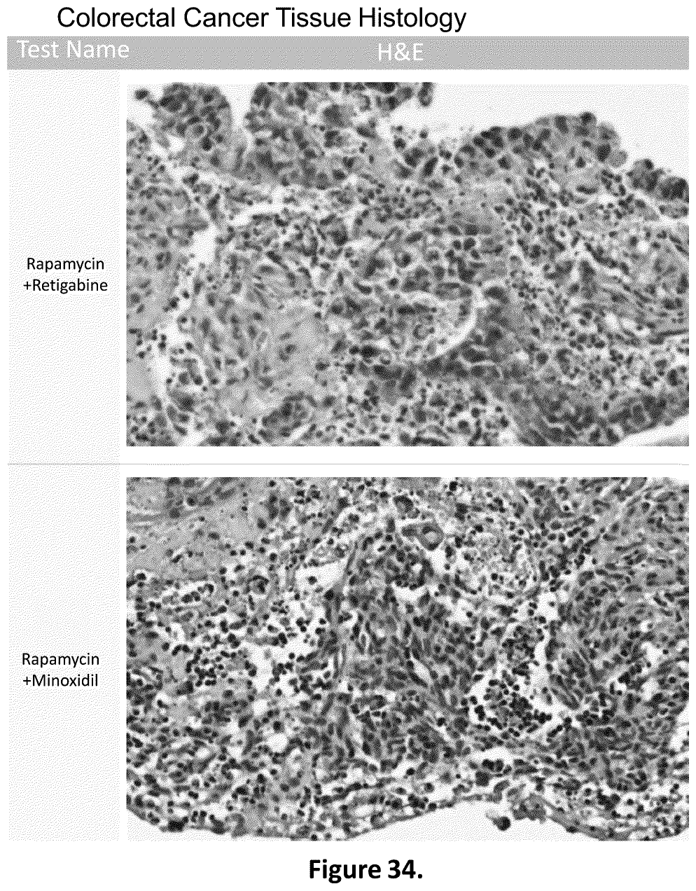

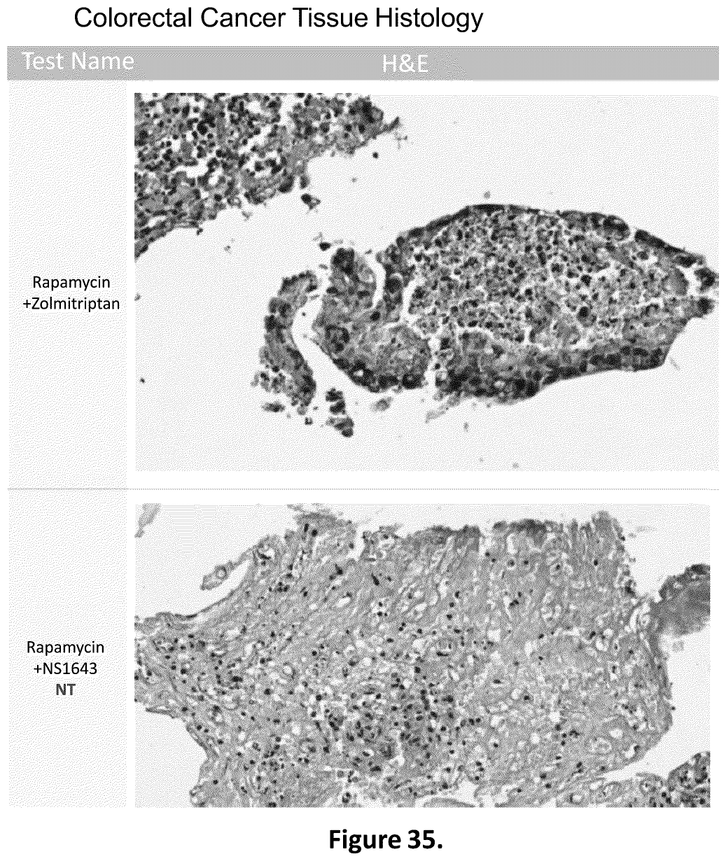

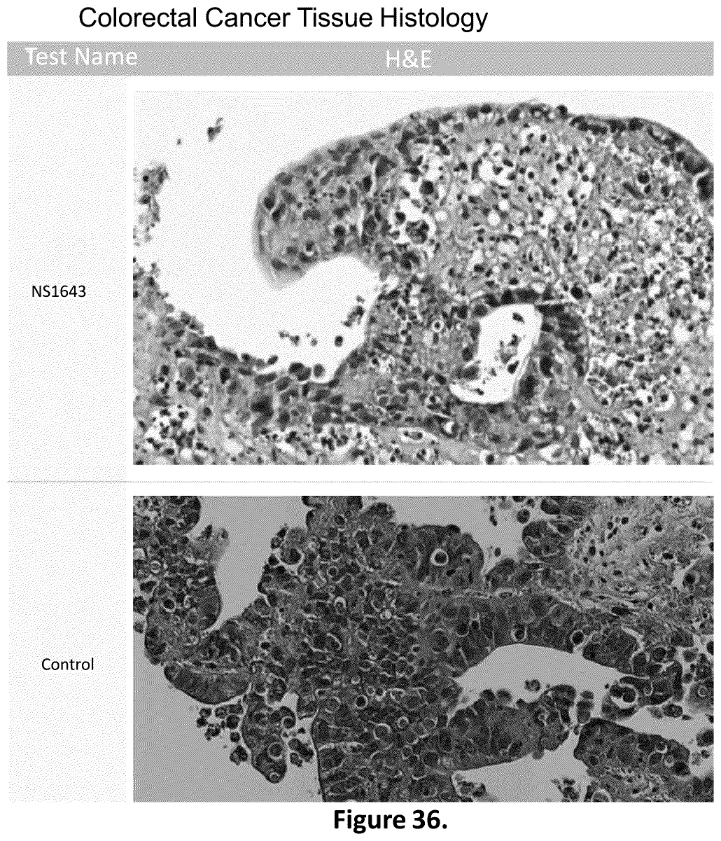

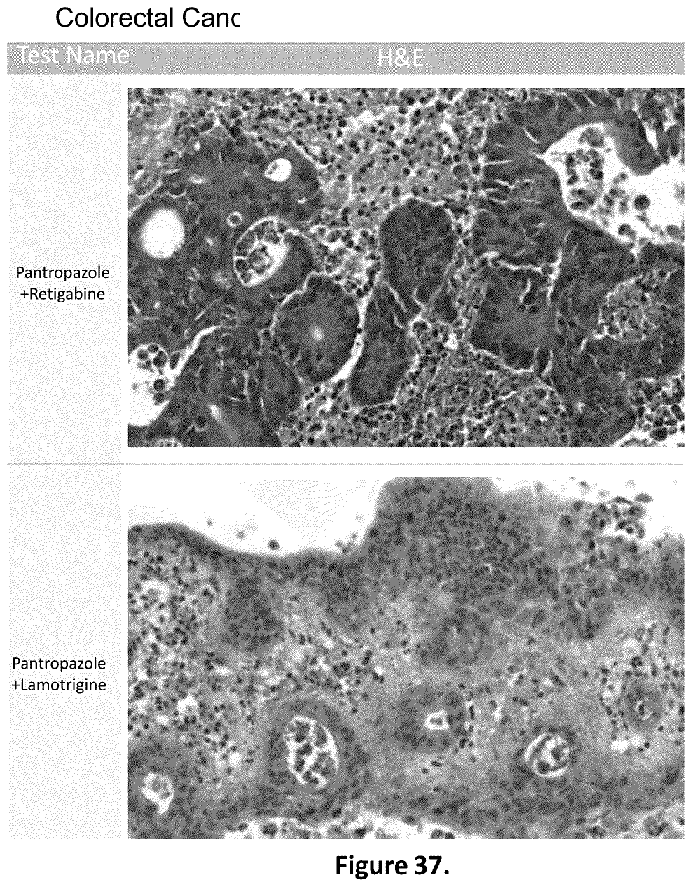

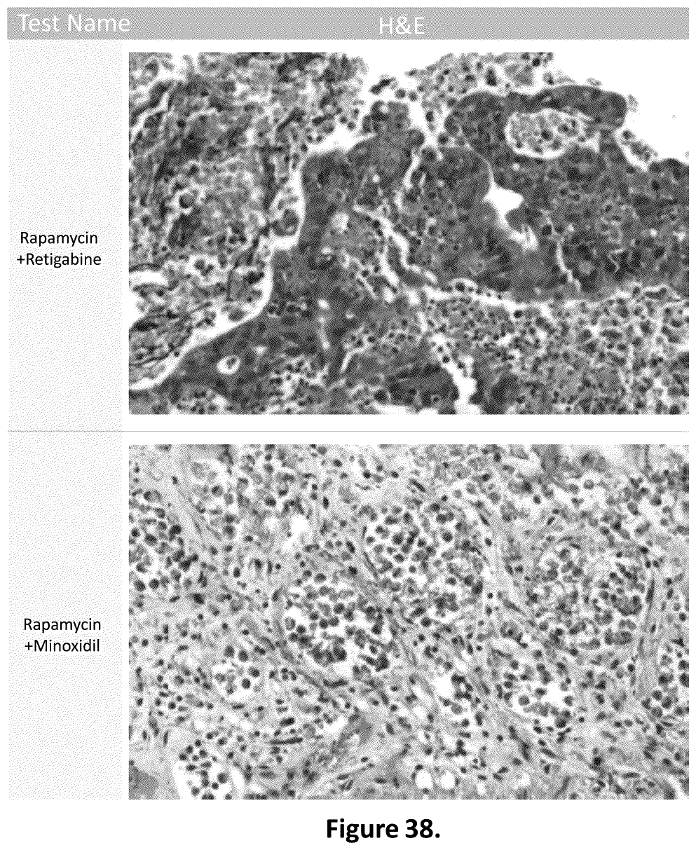

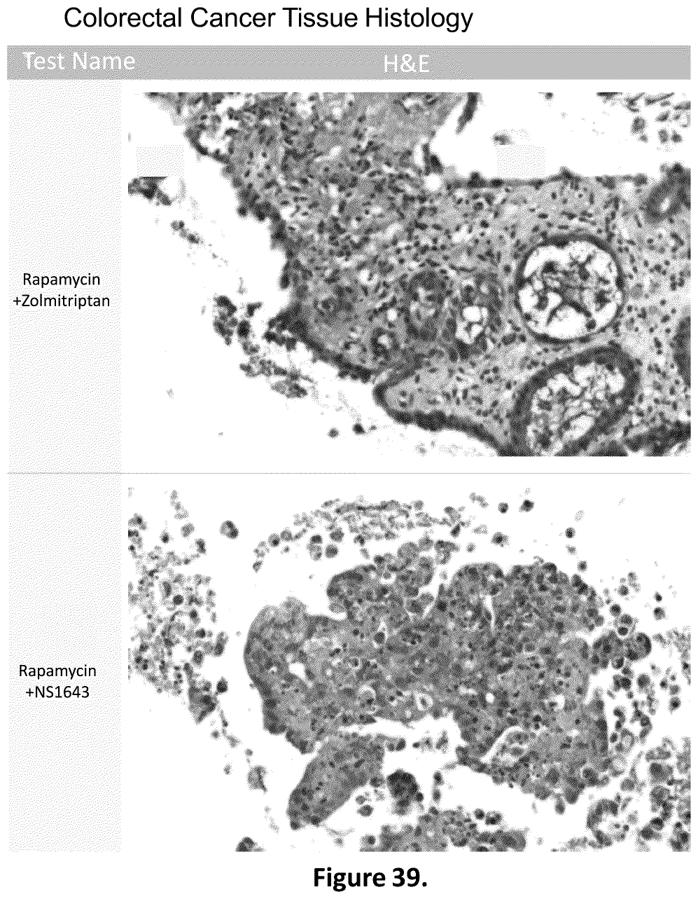

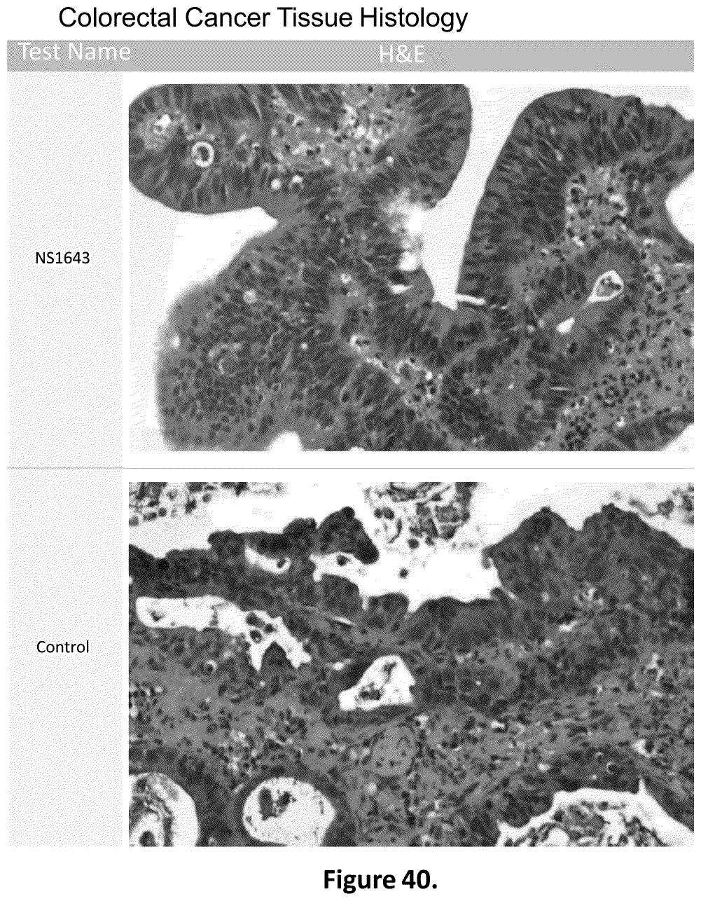

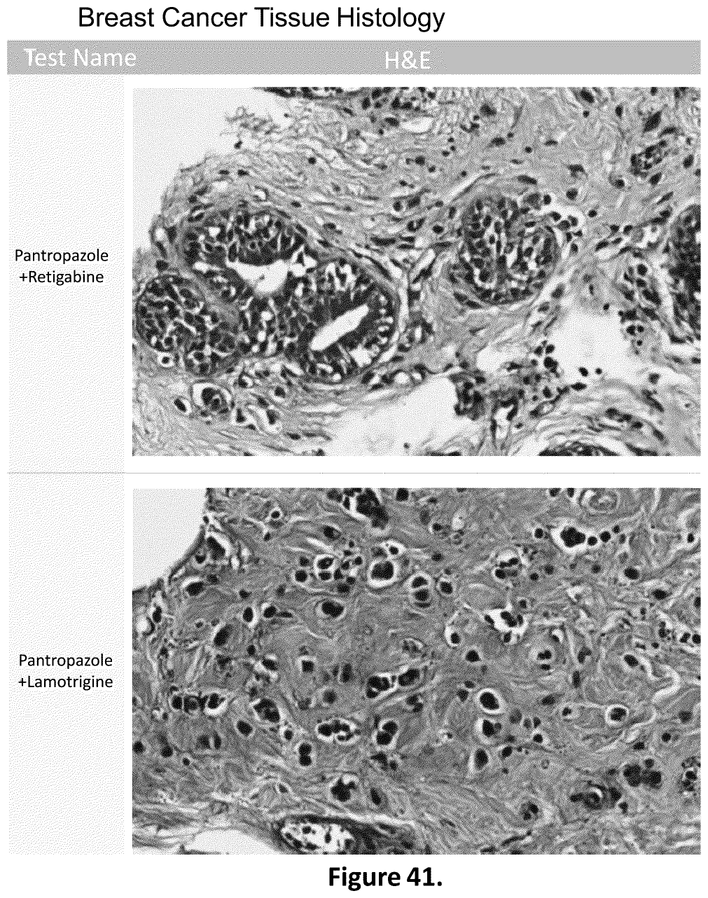

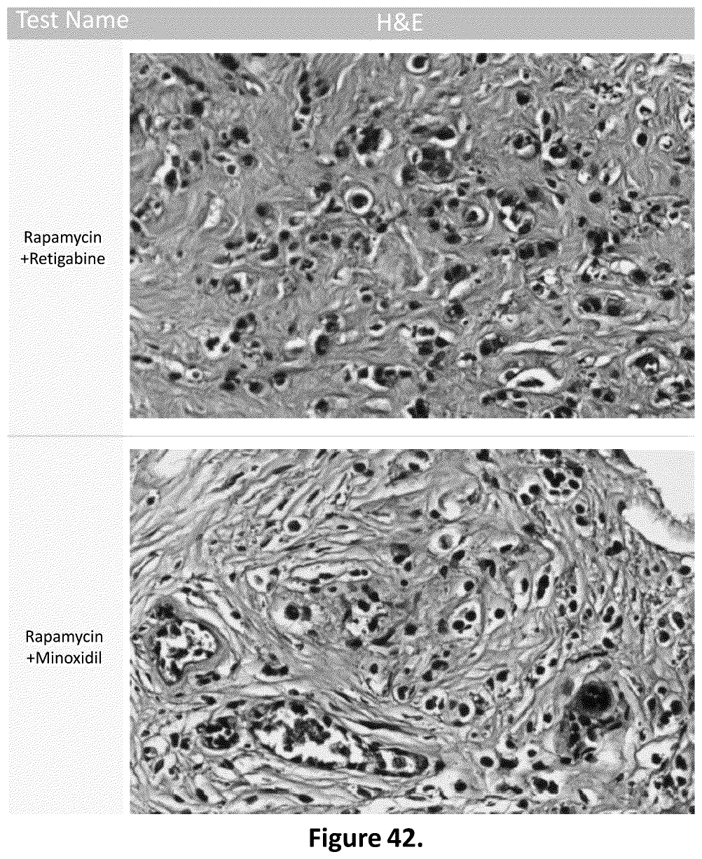

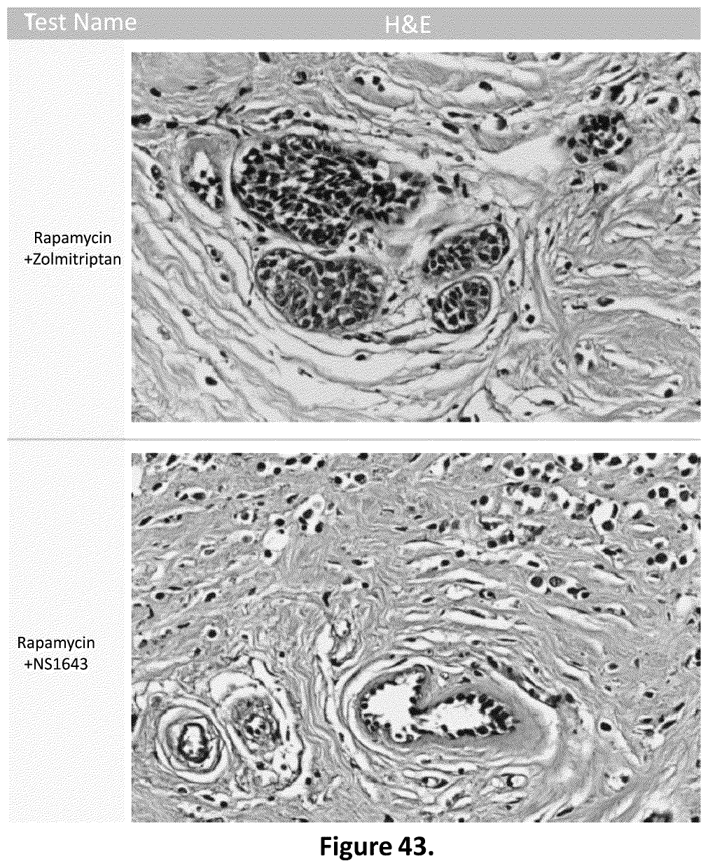

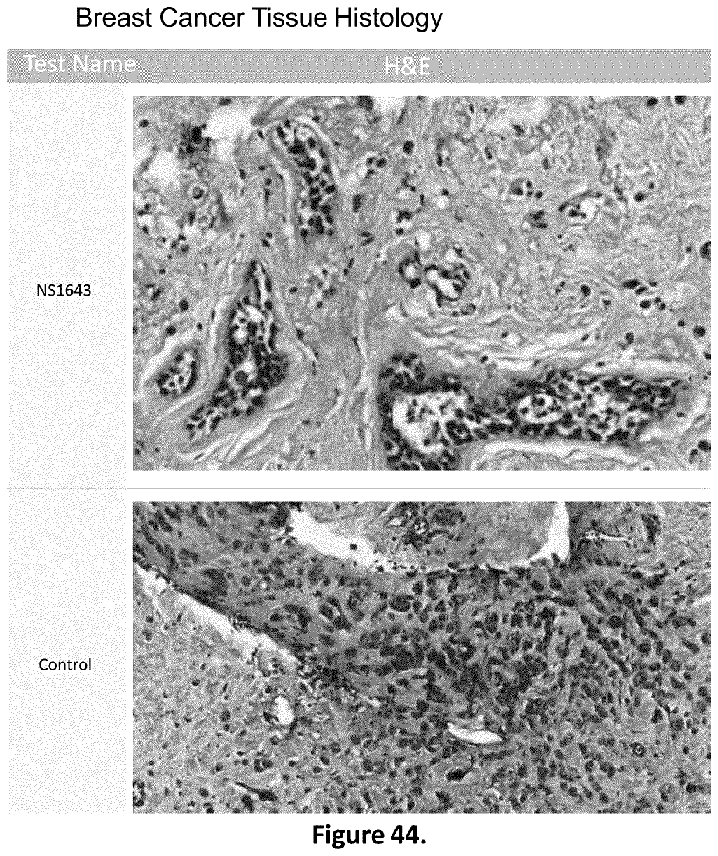

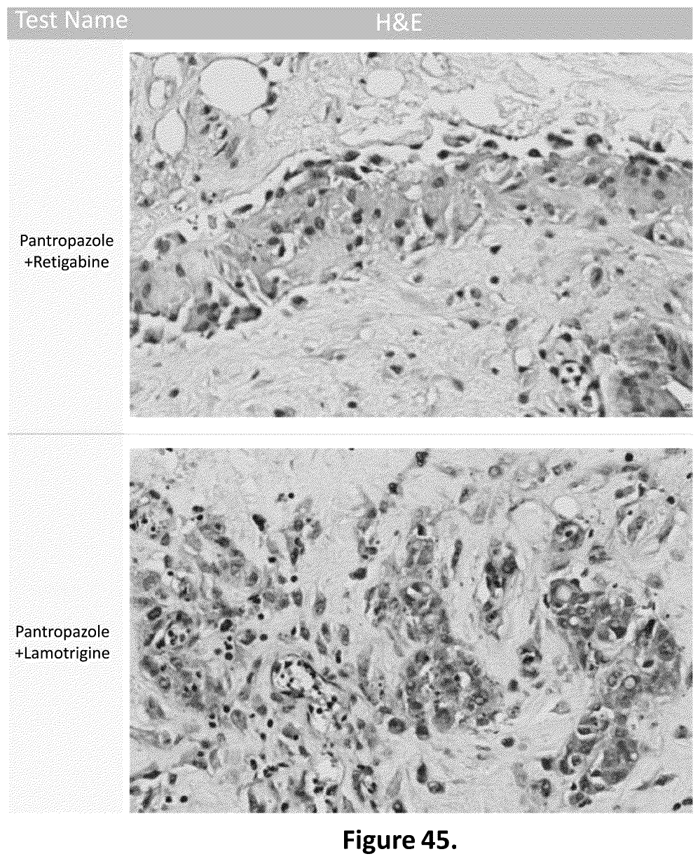

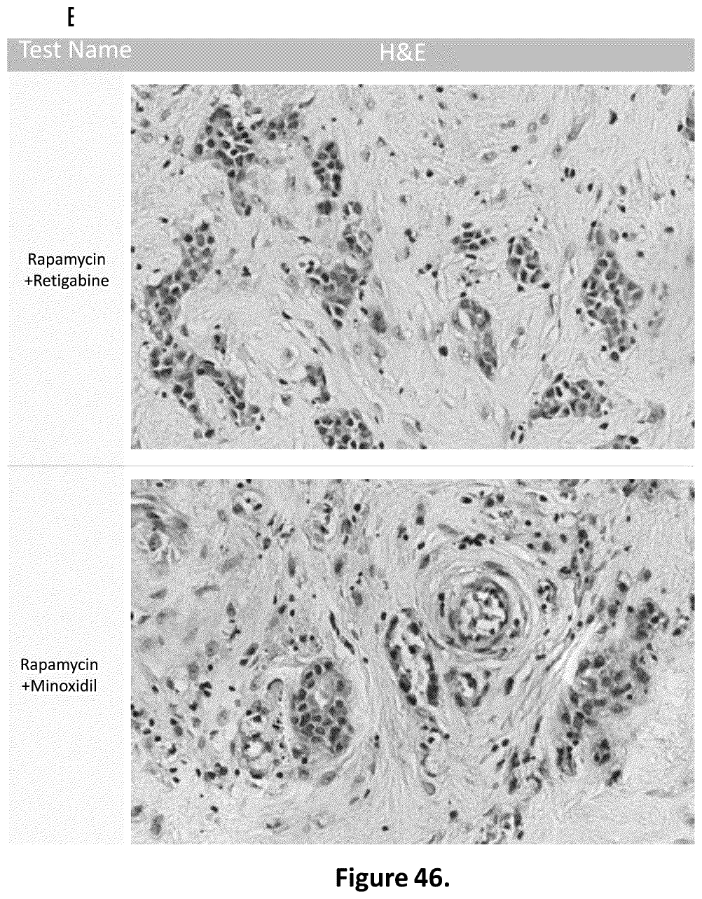

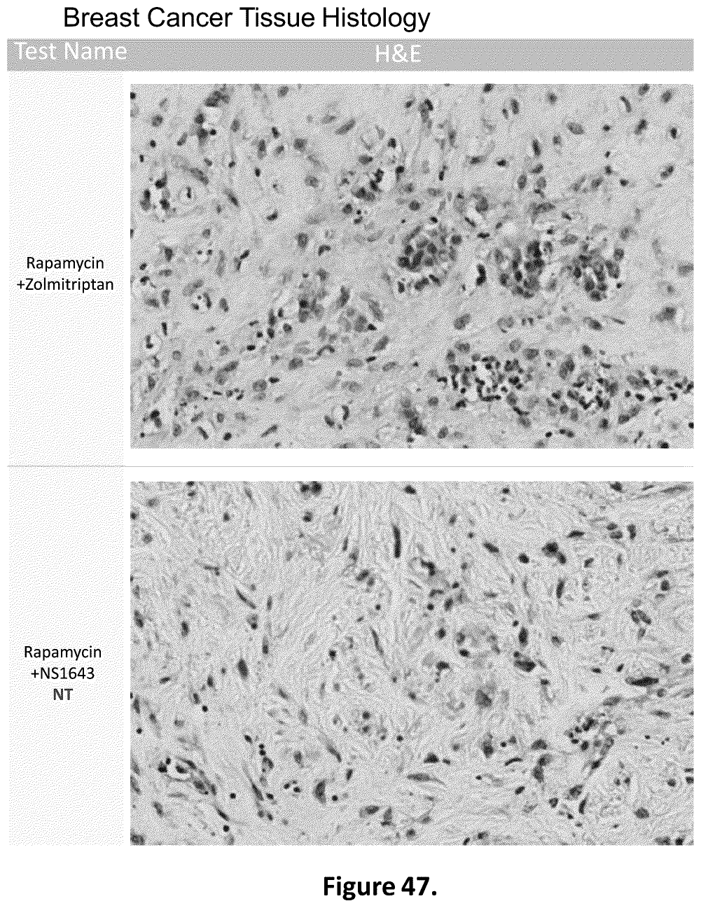

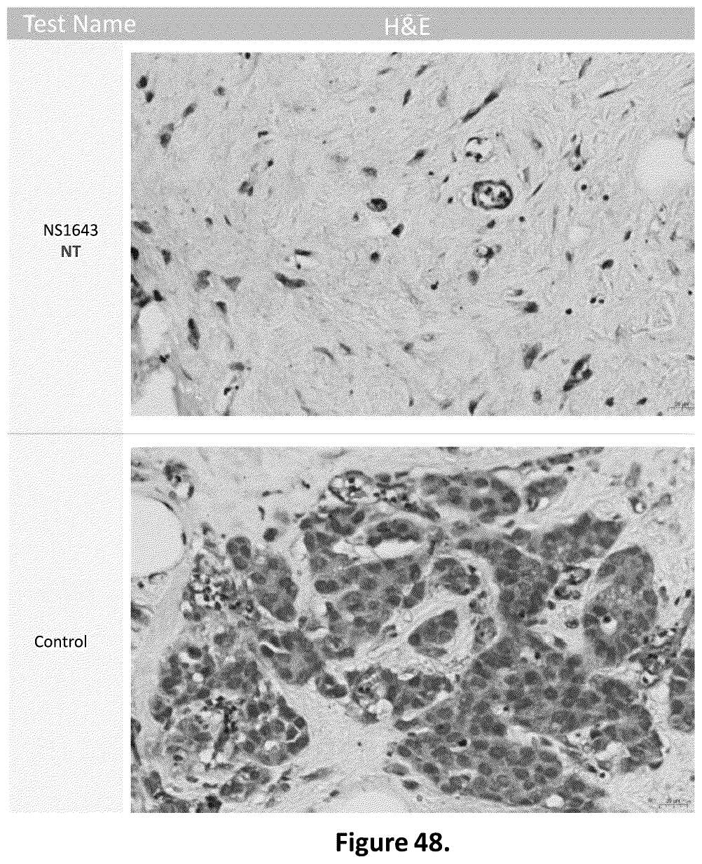

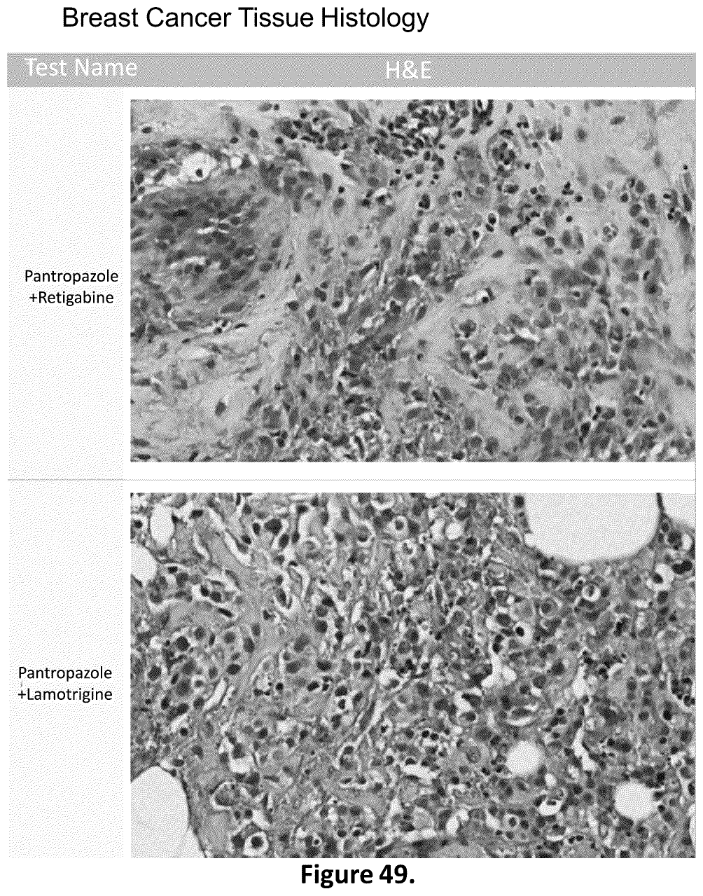

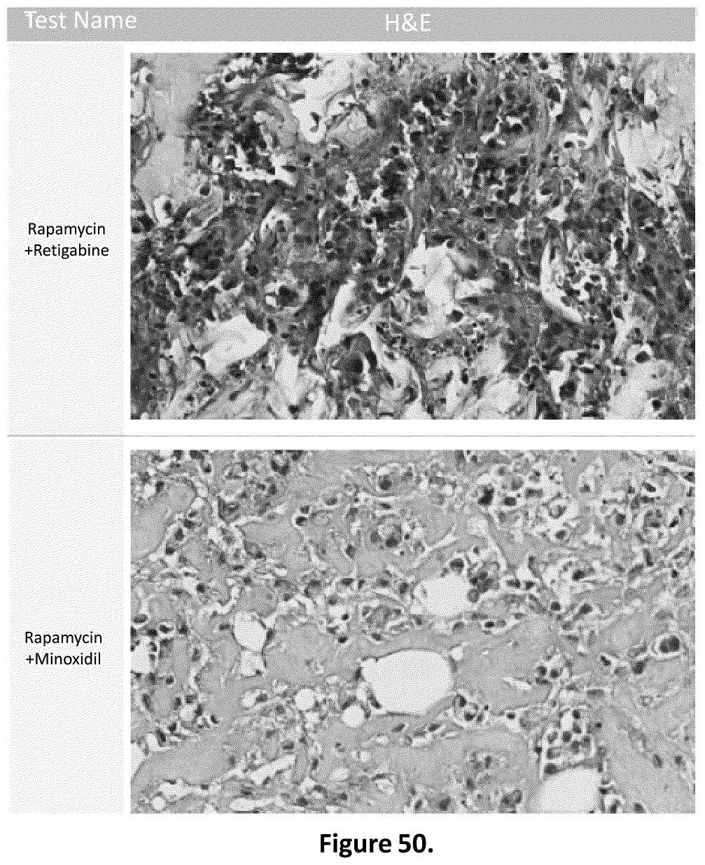

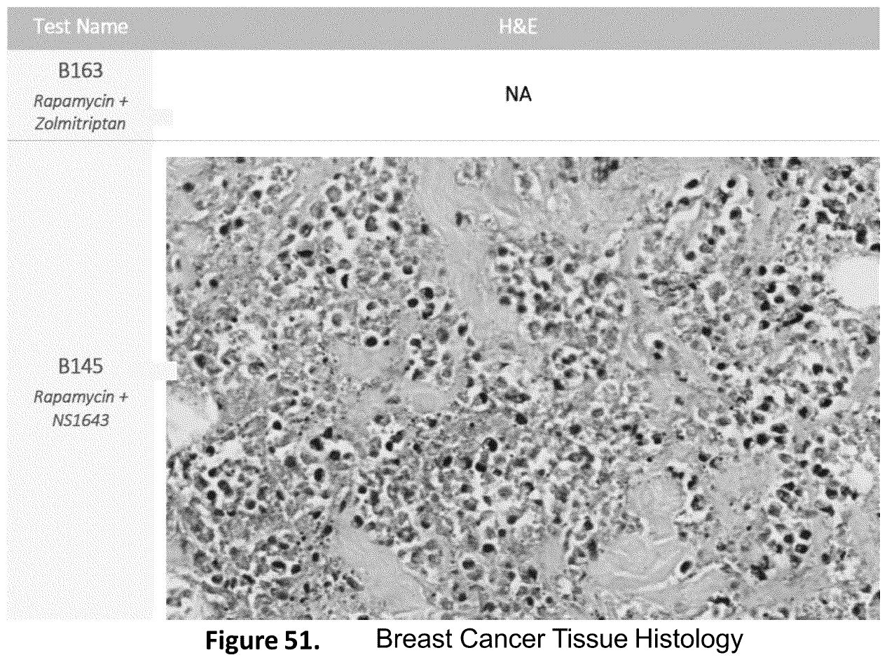

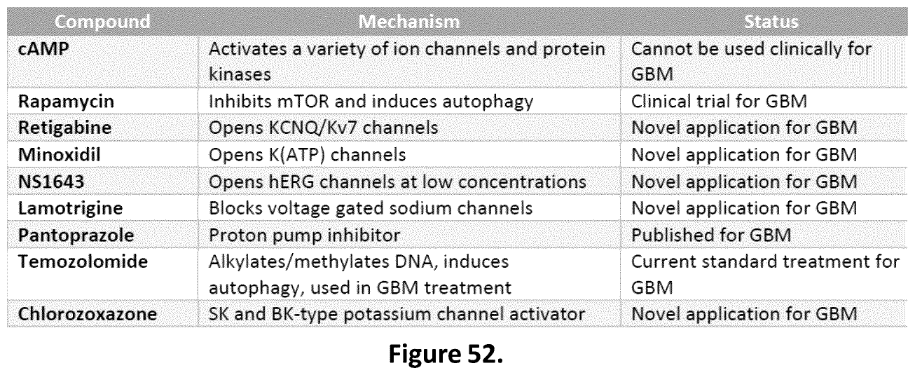

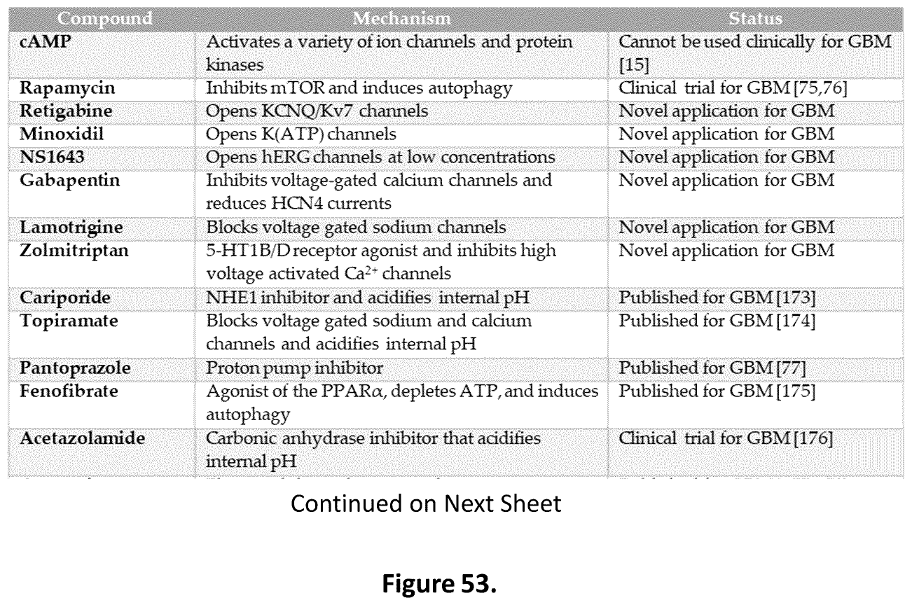

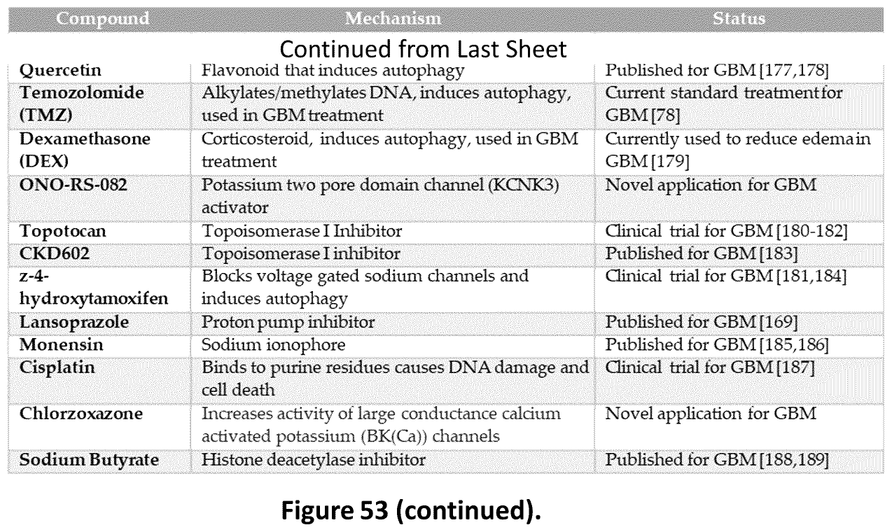

Results of the tumor tissue cytotoxicity assay for the drug treatments on either colorectal cancer or breast cancer tissues are shown in FIGS. 21-51. FIGS. 23-51 show examples of histological results of the tumor tissue cytotoxicity assay for the various drug treatments on both colorectal cancer and breast cancer tissues. Each tissue tested responded best (higher score) to a different drug or drug combination (FIGS. 21-22). There was no single combination that demonstrated strong responses on all samples (FIGS. 21-22). The combination of rapamycin and minoxidil scored 50 and above in four out of six tissues and was above median in five out of six samples (FIGS. 21-22). One colorectal cancer tumor sample and breast cancer tumor sample were very resistant, and no combination demonstrated meaningful impact on these tissues (see FIGS. 21-22). These two samples were resistant to the treatment combining rapamycin and minoxidil (see FIG. 22).

Because each patient's unique tumor may respond differently to therapy (creating a need for personalized cancer treatments), it is not surprising that each of the tested tissues responded best to a different drug treatment. However, in terms of robustness and population trends, the combination of Rapamycin+ Minoxidil was found to be effective in 66% of the tissues that were tested and in 5 out of 6 cases (83%) this treatment scored higher than the median. Only tissues that were generally resistant to various drug treatments, did not respond to this combination.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for treating a cell proliferative disease or disorder in a subject in need thereof, the method comprising administering to the subject an effective amount of one or more potassium channel activators.

2. The method of claim 1, wherein the potassium channel is a KCNQ/Kv7 channel.

3. The method of claim 1 or 2, wherein the one or more potassium channel activators comprise retigabine.

4. The method of any one of the preceding claims, wherein the potassium channel is a K(ATP) channel.

5. The method of any one of the preceding claims, wherein the one or more potassium channel activators comprise minoxidil.

6. The method of any one of the preceding claims, wherein the potassium channel is a hERG channel.

7. The method of any one of the preceding claims, wherein the one or more potassium channel activators comprise NS1643.

8. The method of claim 1, wherein the one or more potassium channel activators comprise retigabine and NS1643.

9. The method of any one of the preceding claims, wherein the potassium channel is a KCNK3 channel.

10. The method of any one of the preceding claims, wherein the one or more potassium channel activators comprise ONO-RS-082.

11. The method of claim 1, wherein the one or more potassium channel activators comprise ONO-RS-082 and NS1643.

12. The method of any one of the preceding claims, wherein the potassium channel is a BK or SK channel.

13. The method of any one of the preceding claims, wherein the one or more potassium channel activators comprise chlorzoxazone.

14. The method of claim 1, wherein the one or more potassium channel activators comprise chlorzoxazone and NS1643.

15. The method of any one of the preceding claims, wherein the cell proliferative disease or disorder is a cancer that expresses the potassium channel selected from brain cancer (e.g., glioblastoma multiforme (GBM)), prostate cancer, breast cancer, lung cancer (e.g., non-small cell lung cancer (NSCLC)), colorectal cancer, bladder cancer, kidney cancer, uterine cancer, melanoma, lymphoma (e.g., Non-Hodgkin lymphoma), leukemia, pancreatic cancer, ovarian cancer, liver and intrahepatic bile duct cancer, oral cavity cancer, and esophageal cancer.

16. The method of any one of the preceding claims, wherein the cell proliferative disease or disorder is GBM.

17. The method of any one of the preceding claims, further comprising administering to the subject an effective amount a mTOR inhibitor before, concurrently with, or after administering to the subject the effective amount of the one or more potassium channel activators.

18. The method of any one of the preceding claims, further comprising administering to the subject an effective amount rapamycin before, concurrently with, or after administering to the subject the effective amount of the one or more potassium channel activators.

19. The method of any one of the preceding claims, further comprising administering to the subject an effective amount of an alkylating agent before, concurrently with, or after administering to the subject the effective amount of the one or more potassium channel activators.

20. The method of any one of the preceding claims, further comprising administering to the subject an effective amount of temozolomide before, concurrently with, or after administering to the subject the effective amount of the one or more potassium channel activators.

21. The method of any one of the preceding claims, further comprising administering to the subject an effective amount a corticosteroid before, concurrently with, or after administering to the subject the effective amount of the one or more potassium channel activators.

22. The method of any one of the preceding claims, further comprising administering to the subject an effective amount of dexamethasone before, concurrently with, or after administering to the subject the effective amount of the one or more potassium channel activators.

23. The method of any one of the preceding claims, further comprising administering to the subject an effective amount of a proton pump inhibitor before, concurrently with, or after administering to the subject the effective amount of the one or more potassium channel activators.

24. The method of any one of the preceding claims, further comprising administering to the subject an effective amount of pantoprazole before, concurrently with, or after administering to the subject the effective amount of the one or more potassium channel activators.

25. The method of any one of the preceding claims, further comprising administering to the subject an effective amount of a sodium channel inhibitor (e.g., a sodium channel blocker) before, concurrently with, or after administering to the subject the effective amount of the one or more potassium channel activators.

26. The method of any one of the preceding claims, further comprising administering to the subject an effective amount of lamotrigine before, concurrently with, or after administering to the subject the effective amount of the one or more potassium channel activators.

27. The method of any one of the preceding claims, further comprising administering to the subject an effective amount of cariporide before, concurrently with, or after administering to the subject the effective amount of the one or more potassium channel activators.

28. The method of any one of the preceding claims, further comprising administering to the subject an effective amount of topiramate before, concurrently with, or after administering to the subject the effective amount of the one or more potassium channel activators.

29. The method of any one of the preceding claims, further comprising administering to the subject an effective amount of a calcium channel inhibitor (e.g., a calcium channel blocker) before, concurrently with, or after administering to the subject the effective amount of the one or more one or more potassium channel activators.

30. The method of any one of the preceding claims, further comprising administering to the subject an effective amount of gabapentin before, concurrently with, or after administering to the subject the effective amount of the one or more potassium channel activators.

31. The method of any one of the preceding claims, further comprising administering to the subject an effective amount of zolmitriptan before, concurrently with, or after administering to the subject the effective amount of the one or more potassium channel activators.

32. The method of any one of the preceding claims, further comprising administering to the subject an effective amount of a peroxisome proliferator-activated receptor alpha (PPARα) activator (e.g., a PPARα agonist) before, concurrently with, or after administering to the subject the effective amount of the one or more potassium channel activators.

33. The method of any one of the preceding claims, further comprising administering to the subject an effective amount of fenofibrate before, concurrently with, or after administering to the subject the effective amount of the one or more potassium channel activators.

34. A method for treating a cell proliferative disease or disorder in a subject in need thereof, the method comprising: (i) administering to the subject an effective amount of a sodium channel inhibitor (e.g., a sodium channel blocker); and (ii) administering to the subject an effective amount of a mTOR inhibitor, wherein the sodium channel inhibitor is administered to the subject before, concurrently with, or after the mTOR inhibitor is administered to the subject.

35. The method of claim 34, wherein the sodium channel is a voltage-gated sodium channel.

36. The method of claim 34, wherein the sodium channel inhibitor is lamotrigine.

37. The method of claim 34, wherein the sodium channel inhibitor is topiramate.

38. The method of claim 34, wherein the sodium channel is NHE1.

39. The method of claim 34, wherein the sodium channel inhibitor is cariporide.

40. The method of any one of claims 34-39, wherein the mTOR inhibitor is rapamycin.

41. The method of any one of claims 34-40, wherein the cell proliferative disease or disorder is a cancer selected from brain cancer (e.g., glioblastoma multiforme (GBM)), prostate cancer, breast cancer, lung cancer (e.g., non-small cell lung cancer (NSCLC)), colorectal cancer, bladder cancer, kidney cancer, uterine cancer, melanoma, lymphoma (e.g., Non-Hodgkin lymphoma), leukemia, pancreatic cancer, ovarian cancer, liver and intrahepatic bile duct cancer, oral cavity cancer, and esophageal cancer.

42. The method of claim 41, wherein the cell proliferative disease or disorder is GBM.

43. A method for treating a cell proliferative disease or disorder in a subject in need thereof, the method comprising: (i) administering to the subject an effective amount of a sodium channel inhibitor (e.g., a sodium channel blocker); and (ii) administering to the subject an effective amount of a proton pump inhibitor, wherein the sodium channel inhibitor is administered to the subject before, concurrently with, or after the proton pump inhibitor is administered to the subject.

44. The method of claim 43, wherein the sodium channel is a voltage-gated sodium channel.

45. The method of claim 43, wherein the sodium channel inhibitor is lamotrigine.

46. The method of claim 43, wherein the sodium channel inhibitor is topiramate.

47. The method of claim 43, wherein the sodium channel is NHE1.

48. The method of claim 43, wherein the sodium channel inhibitor is cariporide.

49. The method of any one of claims 43-48, wherein the proton pump inhibitor is pantoprazole.

50. The method of any one of claims 43-49, wherein the cell proliferative disease or disorder is a cancer selected from brain cancer (e.g., glioblastoma multiforme (GBM)), prostate cancer, breast cancer, lung cancer (e.g., non-small cell lung cancer (NSCLC)), colorectal cancer, bladder cancer, kidney cancer, uterine cancer, melanoma, lymphoma (e.g., Non-Hodgkin lymphoma), leukemia, pancreatic cancer, ovarian cancer, liver and intrahepatic bile duct cancer, oral cavity cancer, and esophageal cancer.

51. The method of claim 50, wherein the cell proliferative disease or disorder is GBM.

52. A method for treating a cell proliferative disease or disorder in a subject in need thereof, the method comprising: (i) administering to the subject an effective amount of a calcium channel inhibitor (e.g., a calcium channel blocker); and (ii) administering to the subject an effective amount of a mTOR inhibitor, wherein the calcium channel inhibitor is administered to the subject before, concurrently with, or after the mTOR inhibitor is administered to the subject.

53. The method of claim 52, wherein the calcium channel is a voltage-activated calcium channel.

54. The method of claim 52, wherein the calcium channel inhibitor is zolmitriptan.

55. The method of any one of claims 52-54, wherein the mTOR inhibitor is rapamycin.

56. The method of any one of claims 52-55, wherein the cell proliferative disease or disorder is a cancer selected from brain cancer (e.g., glioblastoma multiforme (GBM)), prostate cancer, breast cancer, lung cancer (e.g., non-small cell lung cancer (NSCLC)), colorectal cancer, bladder cancer, kidney cancer, uterine cancer, melanoma, lymphoma (e.g., Non-Hodgkin lymphoma), leukemia, pancreatic cancer, ovarian cancer, liver and intrahepatic bile duct cancer, oral cavity cancer, and esophageal cancer.

57. The method of claim 56, wherein the cell proliferative disease or disorder is GBM.

58. A method for treating a cell proliferative disease or disorder in a subject in need thereof, the method comprising: (i) administering to the subject an effective amount of a proton pump inhibitor; and (ii) administering to the subject an effective amount of an alkylating agent, wherein the proton pump inhibitor is administered to the subject before, concurrently with, or after the alkylating agent is administered to the subject.

59. The method of claim 58, wherein the proton pump inhibitor is pantoprazole.

60. The method of claim 58 or 59, wherein the alkylating agent is temozolomide.

61. The method of any one of claims 58-60, wherein the cell proliferative disease or disorder is a cancer selected from brain cancer (e.g., glioblastoma multiforme (GBM)), prostate cancer, breast cancer, lung cancer (e.g., non-small cell lung cancer (NSCLC)), colorectal cancer, bladder cancer, kidney cancer, uterine cancer, melanoma, lymphoma (e.g., Non-Hodgkin lymphoma), leukemia, pancreatic cancer, ovarian cancer, liver and intrahepatic bile duct cancer, oral cavity cancer, and esophageal cancer.

62. The method of claim 61, wherein the cell proliferative disease or disorder is GBM.
